SEQUENCE LISTING (26 sequences)

SEQ ID NO: 1 - protein, 12 aa, synthetic construct
  REGION 1..12: note = spacer (IgG4hinge)
ESKYGPPCPPCP

SEQ ID NO: 2 - DNA, 36 bp, synthetic construct
  source 1..36: mol_type = other DNA
  misc_feature 1..36: note = spacer (IgG4hinge)
gaatctaagtacggaccgccctgccccccttgccct

SEQ ID NO: 3 - protein, 119 aa, synthetic construct
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, synthetic construct
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, synthetic construct
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, synthetic construct
  REGION 1..27: note = CD28 (amino acids 153-179 of Accession No. P10747)
FWVLVVVGGVLACYSLLVTVAFIIFWV

SEQ ID NO: 9 - protein, 66 aa, synthetic construct
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, synthetic construct
  REGION 1..41: note = CD28 (amino acids 180-220 of P10747)
RSKRSRLLHSDYMNMTPRRPGPTRKHYQPYAPPRDFAAYRS

SEQ ID NO: 11 - protein, 41 aa, synthetic construct
  REGION 1..41: note = CD28 (LL to GG)
RSKRSRGGHSDYMNMTPRRPGPTRKHYQPYAPPRDFAAYRS

SEQ ID NO: 12 - protein, 42 aa, synthetic construct
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 13 - protein, 112 aa, synthetic construct
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, synthetic construct
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, synthetic construct
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, synthetic construct
  REGION 1..18: note = T2A
EGRGSLLTCGDVEENPGP

SEQ ID NO: 18 - protein, 22 aa, synthetic construct
  REGION 1..22: note = P2A
GSGATNFSLLKQAGDVEENPGP

SEQ ID NO: 19 - protein, 19 aa, synthetic construct
  REGION 1..19: note = P2A
ATNFSLLKQAGDVEENPGP

SEQ ID NO: 20 - protein, 20 aa, synthetic construct
  REGION 1..20: note = E2A
QCTNYALLKLAGDVESNPGP

SEQ ID NO: 21 - protein, 22 aa, synthetic construct
  REGION 1..22: note = F2A
VKQTLNFDLLKLAGDVESNPGP

SEQ ID NO: 22 - protein, 30 aa, synthetic construct
  REGION 1..30: note = Linker
PGGGSGGGGSGGGGSGGGGSGGGGSGGGGP

SEQ ID NO: 23 - protein, 17 aa, synthetic construct
  REGION 1..17: note = Linker
GSADDAKKDAAKKDGKS

SEQ ID NO: 24 - DNA, 66 bp, synthetic construct
  source 1..66: mol_type = other DNA
  misc_feature 1..66: note = GMCSFR alpha chain signal sequence
atgcttctcctggtgacaagccttctgctctgtgagttaccacacccagcattcctcctgatccca

SEQ ID NO: 25 - protein, 22 aa, synthetic construct
  REGION 1..22: note = GMCSFR alpha chain signal sequence
MLLLVTSLLLCELPHPAFLLIP

SEQ ID NO: 26 - protein, 18 aa, synthetic construct
  REGION 1..18: note = CD8 alpha signal peptide
MALPVTALLLPLALLLHA

(19) United States
(12) Patent Application Publication
(10) Pub. No.: US 2025/0101379 A1
(43) Pub. Date: Mar. 27, 2025

(54) METHODS OF MANUFACTURING CELLULAR COMPOSITIONS

(71) Applicant: Juno Therapeutics, Inc., Seattle, WA (US)

(72) Inventors: Calvin CHAN, Seattle, WA (US); Chin-Wei LIM, Seattle, WA (US)

(73) Assignee: Juno Therapeutics, Inc., Seattle, WA (US)

(21) Appl. No.: 18/833,845

(22) PCT Filed: Jan. 27, 2023
(86) PCT No.: PCT/US2023/061510

§ 371 Date: Jul. 26, 2024

Related U.S. Application Data
(60) Provisional application No. 63/304,502, filed on Jan. 28, 2022.
(60) Provisional application No. 63/438,764, filed on Jan. 12, 2023.

(51) Int. Cl. C12N 5/0783; C07K 14/725

(52) U.S. Cl. CPC C12N 5/0636; C07K 14/7051; C07K 2317/55; C07K 2319/03; C12N 2501/2302; C12N 2501/2307; C12N 2501/2315; C12N 2501/51; C12N 2501/515; C12N 2510/00

(57) ABSTRACT

Provided are methods of continuous counterflow centrifugation for the manufacturing of cell compositions, including for the production of T cell therapies including cells that express recombinant receptors such as chimeric antigen receptors (CARs).

### CROSS-REFERENCE TO RELATED APPLICATIONS

This application claims priority to U.S. Provisional Application No. 63/304,502, filed Jan. 28, 2022, and U.S. Provisional Application No. 63/438,764, filed Jan. 12, 2023, the contents of each of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042021040SeqList.xml, created Jan. 23, 2023, which is 35,102 bytes in size. The information in the electronic format of the Sequence Listing is incorporated by reference in its entirety.

### FIELD

The present disclosure relates in some aspects to methods of continuous counterflow centrifugation in the processing and manufacturing of cell compositions, including engineered cell compositions. In some aspects, methods of continuous counterflow centrifugation enrich cell compositions for viable cells, such as viable T cells. Also provided are the resulting cell compositions, such as those comprising T cells genetically engineered to express an antigen receptor. In some aspects, the transduction of cell compositions, such as compositions comprising a population of lymphocytes, is carried out by continuous counterflow centrifugation. In some aspects, the disclosure provides methods for the transduction of cell populations that involve continuous counterflow centrifugation of lymphocytes and viral vector particles, thereby producing a composition containing transduced cells. In some embodiments, the provided cells and compositions can be used in methods of adoptive cell therapy.

### BACKGROUND

Various strategies are available for enriching and/or transducing T cell populations in vitro, including for enriching and transducing antigen-specific T cells in vitro for use in adoptive cellular immunotherapy or cancer therapy. Improved strategies are needed for enriching and/or transducing cell populations in vitro, including for large-scale research, diagnostic, and therapeutic purposes. Provided are methods and related compositions that meet such needs.

### SUMMARY

Provided herein is a method for producing a composition of genetically engineered T cells comprising (a) applying a first centrifugal force and a first flow rate to a cell composition comprising T cells in a conical fluid enclosure of a centrifuge system to produce a fluidized bed of cells; (b) loading a viral vector particle into the conical fluid enclosure, thereby generating an input composition comprising the cell composition and the viral vector particle; and (c) applying a second centrifugal force and a second flow rate to the input composition, wherein the second centrifugal force and second flow rate recirculate the viral vector particle in a fluid path of the centrifuge system, thereby generating genetically engineered T cells. In some embodiments, the centrifuge system is a continuous counterflow centrifuge system.

In some embodiments, the loading of the viral vector particle is carried out during at least a portion of the applying in (a). In some embodiments, the loading of the viral vector particle is carried out during at least a portion of the applying in (c).

Also provided herein is a method for producing a composition of genetically engineered T cells comprising (a) applying a first centrifugal force and a first flow rate to an input composition comprising (i) a viral vector particle and (ii) a cell composition comprising T cells in a conical fluid enclosure of a centrifuge system to produce a fluidized bed of cells; and (b) applying a second centrifugal force and a second flow rate to the input composition in the conical fluid enclosure, wherein the second centrifugal force and second flow rate recirculate the viral vector particle in a fluid path of the centrifuge system, thereby generating genetically engineered T cells. In some embodiments, the centrifuge system is a continuous counterflow centrifuge system.

In some embodiments, the method comprises loading the cell composition and the viral vector particle into the conical fluid enclosure, thereby generating the input composition. In some embodiments, the loading of the cell composition is before, during, and/or after the loading of the viral vector particle. In some embodiments, the loading of the cell composition is before the loading of the viral vector particle. In some embodiments, the loading of the cell composition is during the loading of the viral vector particle. In some embodiments, the loading of the cell composition is after the loading of the viral vector particle. In some embodiments, the loading of the cell composition and/or the loading of the viral vector particle is performed prior to and/or during the applying in (a). In some embodiments, the loading of the cell composition is performed prior to and/or during the applying in (a). In some embodiments, the loading of the cell composition is performed prior to the applying in (a). In some embodiments, the loading of the cell composition is performed during the applying in (a). In some embodiments, the loading of the cell composition is performed prior to and during the applying in (a). In some embodiments, the loading of the viral vector particle is performed prior to the applying in (a). In some embodiments, the loading of the viral vector particle is performed during the applying in (a). In some embodiments, the loading of the viral vector particle is performed prior to and during the applying in (a).

In some embodiments, the method comprises applying a third centrifugal force and a third flow rate to the genetically engineered T cells in the conical fluid enclosure of the centrifuge system to produce an output composition comprising the genetically engineered T cells.

In some embodiments, the percentage of viable T cells in the output composition is greater than the percentage of viable T cells in the input composition. In some embodiments, the percentage of viable T cells in the output composition is at least about 5% greater, at least about 10% greater, at least about 15% greater, at least about 20% greater, or at least about 25% greater than the percentage of viable T cells in the input composition. In some embodiments, the percentage of viable T cells in the output composition is about 5% greater than the percentage of viable T cells in the input composition. In some embodiments, the percentage of viable T cells in the output composition is about 10% greater than the percentage of viable T cells in the input composition. In some embodiments, the percentage of viable T cells in the output composition is about 15% greater than the percentage of viable T cells in the input composition.

In some embodiments, at least or at least about 5%, at least or at least about 10%, at least or at least about 15%, at least or at least about 20%, at least or at least about 25%, at least or at least about 30% of the T cells in the output composition are transduced with the viral vector particle. In some embodiments, at least about 20% of the T cells in the output composition are transduced with the viral vector particle. In some embodiments, at least about 25% of the T cells in the output composition are transduced with the viral vector particle. In some embodiments, at least about 30% of the T cells in the output composition are transduced with the viral vector particle. In some embodiments, at least about 35% of the T cells in the output composition are transduced with the viral vector particle. In some embodiments, at least about 40% of the T cells in the output composition are transduced with the viral vector particle.

In some embodiments, the first centrifugal force is between about 2,000 G and about 4,000 G. In some embodiments, the first flow rate is between about 5 mL/min and about 15 mL/min. In some embodiments, the first centrifugal force and the first flow rate are applied to the cell composition or the input composition for about 15 seconds, about 30 seconds, about 45 seconds, or about 60 seconds. In some embodiments, the first centrifugal force and the first flow rate are applied to the cell composition or the input composition for about 30 seconds.

In some embodiments, the second centrifugal force is between about 500 G and about 1,500 G. In some embodiments, the second flow rate is between about 25 mL/min and about 30 mL/min. In some embodiments, the second centrifugal force and the second flow rate are applied to the input composition for at least about 15 minutes, at least about 30 minutes, at least about 45 minutes, at least about 60 minutes, at least about 75 minutes, or at least about 90 minutes. In some embodiments, the second centrifugal force and the second flow rate are applied to the input composition for about 30 minutes.

In some embodiments, the second centrifugal force is between about 500 G and about 1,500 G. In some embodiments, the second flow rate is between about 10 mL/min and about 100 mL/min. In some embodiments, the second centrifugal force and the second flow rate are applied to the input composition for at least about 15 minutes, at least about 30 minutes, at least about 45 minutes, at least about 60 minutes, at least about 75 minutes, or at least about 90 minutes. In some embodiments, the second centrifugal force and the second flow rate are applied to the input composition for about 30 minutes.

In some embodiments, the second centrifugal force is between about 100 G and about 2,000 G. In some embodiments, the second flow rate is between about 10 mL/min and about 100 mL/min. In some embodiments, the second centrifugal force and the second flow rate are applied to the input composition for at least about 15 minutes, at least about 30 minutes, at least about 45 minutes, at least about 60 minutes, at least about 75 minutes, or at least about 90 minutes. In some embodiments, the second centrifugal force and the second flow rate are applied to the input composition for about 30 minutes.

In some embodiments, the ratio of the first centrifugal force to the first flow rate is between about 200 and about 400. In some embodiments, the ratio of the first centrifugal force to the first flow rate is about 300. In some embodiments, the first centrifugal force is about 3,000 G and the first flow rate is about 10 mL/min. In some embodiments, the ratio of the second centrifugal force to the second flow rate is between about 20 and about 100, between about 25 and about 85, or between about 30 and about 65. In some embodiments, the ratio of the second centrifugal force to the second flow rate is about 35. In some embodiments, the second centrifugal force is about 1,000 G and the second flow rate is about 28.5 mL/min. In some embodiments, the ratio of the second centrifugal force to the second flow rate is about 62.5.

In some embodiments, the second centrifugal force is about 625 G and the second flow rate is about 10 mL/min.

In some embodiments, the ratio of the second centrifugal force to the second flow rate is about 30. In some embodiments, the second centrifugal force is about 300 G and the second flow rate is about 10 mL/min.

In some embodiments, the third centrifugal force is between about 2,000 G and about 3,000 G. In some embodiments, the third flow rate is between about 15 mL/min and about 25 mL/min. In some embodiments, the ratio of the third centrifugal force to the third flow rate is between about 100 and about 150. In some embodiments, the ratio of the third centrifugal force to the third flow rate is about 125. In some embodiments, the third centrifugal force is about 2,500 G and the third flow rate is about 20 mL/min.

In some embodiments, prior to the applying the third centrifugal force and the third flow rate, the method comprises subjecting the genetically engineered T cells to one or more washing steps. In some embodiments the one or more washing steps comprise media exchange. In some embodiments, the one or more washing steps are carried out at the second centrifugal force and the second flow rate.

In some embodiments, the method comprises incubating the T cells of the cell composition under stimulating conditions prior to the applying in (a). In some embodiments, the T cells of the cell compositions are incubated under stimulating conditions prior to the applying in (a). In some embodiments, the cell composition comprises activated cells. In some embodiments, the stimulating conditions comprise the presence of a stimulatory reagent that is capable of activating one or more intracellular signaling domains of one or more components of a TCR complex and one or more intracellular signaling domains of one or more costimulatory molecules. In some embodiments, the stimulatory reagent comprises (i) a primary agent that specifically binds to a member of a TCR complex, optionally that specifically binds to CD3; and (ii) a secondary agent that specifically binds to a T cell costimulatory molecule. In some embodiments, the primary agent specifically binds to CD3. In some embodiments, the secondary agent specifically binds to a costimulatory molecule selected from CD28, CD137 (4-1-BB), OX40, and ICOS. In some embodiments, the secondary agent specifically binds to CD28.

In some embodiments, at least one of the primary and secondary agents comprises an antibody or an antigen-binding fragment thereof. In some embodiments, the primary agent and the secondary agent each comprises an antibody or an antigen-binding fragment thereof. In some embodiments, the primary agent is an anti-CD3 antibody or an antigen-binding fragment thereof. In some embodiments, the secondary agent is an anti-CD28 antibody or an antigen-binding fragment thereof. In some embodiments, the primary agent is an anti-CD3 antibody or an antigen-binding fragment thereof and the secondary agent is an anti-CD28 antibody or an antigen-binding fragment thereof. In some embodiments, the primary agent and the secondary agent are each present or attached on the surface of a solid support. In some embodiments, the solid support is or comprises a bead. In some embodiments, the solid support is a paramagnetic bead with surface attached anti-CD3 and anti-CD28 antibodies. In some embodiments, the primary agent and the secondary agent are reversibly bound on the surface of an oligomeric particle reagent comprising a plurality of streptavidin molecules or streptavidin mutein molecules. In some embodiments, the streptavidin molecules or the streptavidin mutein molecules bind to or are capable of binding to biotin, avidin, a biotin analog or a biotin mutein, an avidin analog or an avidin mutein and/or a biologically active fragment thereof. In some embodiments, the primary agent comprises an anti-CD3 Fab and the secondary agent comprises an anti-CD28 Fab.

In some embodiments, the stimulating conditions comprise the presence of one or more recombinant cytokines. In some embodiments, the stimulating conditions comprise the presence of one or more of recombinant IL-2, IL-7, and IL-15.

In some embodiments, the method comprises collecting the output composition. In some embodiments, the output composition is collected.

In some embodiments, the method comprises incubating the genetically engineered T cells of the collected output composition. In some embodiments, the genetically engineered T cells of the collected output composition are incubated. In some embodiments, the genetically engineered T cells of the collected output composition are incubated immediately following the collecting for at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days.

In some embodiments, the percentage of viable T cells in the collected output composition about 1 day after, about 2 days after, about 3 days after, about 4 days after, about 5 days after, about 6 days after, about 7 days after, about 8 days after, about 9 days after, or about 10 days after collection is greater than the percentage of viable T cells in the input composition. In some embodiments, the percentage of viable T cells in the collected output composition about 1 day after collection is greater than the percentage of viable T cells in the input composition. In some embodiments, the percentage of viable T cells in the collected output composition about 5 days after collection is greater than the percentage of viable T cells in the input composition.

In some embodiments, the method comprises cryopreserving the collected output composition and/or the collected output composition is cryopreserved, thereby generating a cryopreserved composition. In some embodiments, the method comprises cryopreserving the collected output composition, thereby generating a cryopreserved composition. In some embodiments, the collected output composition is cryopreserved, thereby generating a cryopreserved composition. In some embodiments, the cryopreserved composition is thawed to produce a thawed composition, and the percentage of viable T cells in the thawed composition is greater than the percentage of viable T cells in the input composition. In some embodiments, the percentage of viable T cells in the thawed composition is at least about 5% greater, at least about 10% greater, at least about 15% greater, at least about 20% greater, at least about 25% greater, or at least about 30% greater than the percentage of viable T cells in the input composition.

In some embodiments, the input composition comprises T cells having an average diameter of greater than or greater than about 6 microns, greater than or greater than about 6 microns, greater than or greater than about 7 microns, greater than or greater than about 8 microns, greater or greater than about 9 microns, greater or greater than about 10 microns, or greater or greater than about 11 microns.

In some embodiments, the input composition comprises between about 1×10⁶ total T cells and about 2×10⁹ total T cells. In some embodiments, the input composition comprises at least about 1×10⁸ total T cells, at least about 2×10⁸ total T cells, at least about 3×10⁸ total T cells, at least about 4×10⁸ total T cells, at least about 5×10⁸ total T cells, at least about 6×10⁸ total T cells, at least about 7×10⁸ total T cells, at least about 8×10⁸ total T cells, at least about 7×10⁸ total T cells, at least about 8×10⁸ total T cells, at least about 9×10⁸ total T cells, at least about 1×10⁹ total T cells, at least about 1.25×10⁹ total T cells, at least about 1.50×10⁹ total T cells, or at least about 1.75×10⁹ total T cells. In some embodiments, the volume of the input composition is between about 5 ml and about 20,000 ml, between about 10 mL and about 2,000 mL, between about 15 mL and about 1,000 mL, between about 20 mL and about 500 mL, between about 25 mL and about 100 mL, or between about 30 mL and about 60 mL. In some embodiments, the volume of the input composition is between about 30 mL and about 60 mL. In some embodiments, the volume of the input composition is about 30 mL. In some embodiments, the volume of the input composition is about 60 mL.

In some embodiments, the volume of the output composition is between about 2.5 mL and about 60 mL, between about 5 mL and about 40 mL, or between about 10 mL and about 20 mL. In some embodiments, the volume of the output composition is about 5 mL, about 10 mL, about 15 mL, about 20 mL, about 25 mL, about 30 mL, about 35 mL, about 40 mL, about 45 mL, about 50 mL, about 55 mL, or about 60 mL.

In some embodiments, one or more steps of the method are automated. In some embodiments, the one or more steps of the method are automated by the centrifuge system or a component thereof.

In some embodiments, the viral vector particle comprises a heterologous nucleic acid encoding a recombinant molecule. In some embodiments, the recombinant molecule is a chemokine, a chemokine receptor, a cytokine, a cytokine receptor, an antigen receptor (e.g., a CAR or a TCR), or a combination thereof. In some embodiments, the recombinant molecule is an antigen receptor. In some embodiments, the antigen receptor is a transgenic T cell receptor (TCR). In some embodiments, is a chimeric antigen receptor (CAR).

In some embodiments, the chimeric antigen receptor (CAR) comprises an extracellular antigen-recognition domain that specifically binds to a target antigen and an intracellular signaling domain comprising an immunoreceptor tyrosine-based activation motif (ITAM). In some embodiments, the intracellular signaling domain comprises an intracellular domain of a CD3-zeta (CD3ζ) chain. In some embodiments, the CAR further comprises a transmembrane domain linking the extracellular domain and the intracellular signaling domain. In some embodiments, the transmembrane domain comprises a transmembrane portion of CD28. In some embodiments, the intracellular signaling domain further comprises an intracellular signaling domain of a T cell costimulatory molecule. In some embodiments, the T cell costimulatory molecule is selected from the group consisting of CD28 and 4-1BB. In some embodiments, the CAR is recombinantly expressed. In some embodiments, the CAR is expressed from a vector. In some embodiments, the CAR is expression from a γ-retroviral vector or a lentiviral vector. In some embodiments, the CAR is expressed from a lentiviral vector.

In some embodiments, the viral vector particle is a retroviral vector particle. In some embodiments, the retroviral vector particle is a γ-retroviral vector. In some embodiments, the retroviral vector particle is a lentiviral vector particle.

In some embodiments, the antigen receptor specifically binds to an antigen associated with a disease or a condition. In some embodiments, the disease or condition is a cancer, an autoimmune disease or disorder, and/or an infectious disease. In some embodiments, the disease or condition is a cancer. In some embodiments, the T cells are primary T cells, optionally from a human subject.

Also provided herein is a method for enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells, and (b) applying a second centrifugal force and a second flow rate to the cell composition, wherein the second centrifugal force and second flow rate recirculate cells of the cell composition in a fluid path of the centrifuge system, thereby elutriating out of the conical fluid enclosure a waste fraction of the cell composition that has a higher percentage of nonviable T cells than the percentage of nonviable T cells in the cell composition and producing within the conical fluid enclosure an enriched composition that has a higher percentage of viable T cells than the percentage of viable T cells in the cell composition. In some embodiments, the centrifuge system is a continuous counterflow centrifuge system.

In some embodiments, (i) the first centrifugal force is between about 1,000 G and about 4,000 G; and (ii) the first flow rate is between about 5 mL/min and about 15 mL/min.

In some embodiments, the ratio of the first centrifugal force (in G) to the first flow rate (in mL/min) is between about 200 and about 500. In some embodiments, the ratio of the first centrifugal force (in G) to the first flow rate (in mL/min) is between about 200 and about 400.

In some embodiments, the first centrifugal force and the first flow rate are applied to the cell composition for at least 30 seconds.

In some embodiments, the second centrifugal force is between about 350 G and about 4,000 G; and (ii) the second flow rate is between about 5 mL/min and about 100 mL/min.

In some embodiments, the second centrifugal force is between about 350 G and 3,000 G. In some embodiments, the second centrifugal force is between about 1,500 G and about 3,000 G. In some embodiments, the second centrifugal force is between about 500 G and about 1,500 G.

In some embodiments, the second flow rate is between about 65 mL/min and about 100 mL/min.

In some embodiments, the second flow rate is between about 10 mL/min and about 65 mL/min. In some embodiments, the second flow rate is between about 10 mL/min and about 35 mL/min.

In some embodiments, the second flow rate is between about 25 mL/min and about 30 mL/min.

In some embodiments, the ratio of the second centrifugal force (in G) to the second flow rate (in mL/min) is between about 30 and about 70. In some embodiments, the ratio of the second centrifugal force (in G) to the second flow rate (in mL/min) is between about 30 and about 40.

In some embodiments, the T cells have a mean diameter of about 9 μm to about 20 μm. In some embodiments, the T cells have a mean diameter of less than 9 μm. In some embodiments, the T cells have a mean diameter of about 6 μm to about 9 μm.

In some embodiments, the T cells have a mean diameter of about 9 μm to about 20 μm. In some embodiments, the T cells have a mean diameter of about 9 μm to about 20 μm, and the ratio of the second centrifugal force (in G) to the second flow rate (in mL/min) is between about 30 and about 70.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm.

In some embodiments, the T cells have a mean diameter of less than 9 μm, and the ratio of the second centrifugal force (in G) to the second flow rate (in mL/min) is between about 30 and about 40.

Also provided herein is a method of enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells, wherein (i) the first centrifugal force is between about 2,000 G and about 4,000 G; and (ii) the first flow rate is between about 5 mL/min and about 15 mL/min; and (b) applying a second centrifugal force and a second flow rate to the cell composition, wherein the second centrifugal force and second flow rate recirculate cells of the cell composition in a fluid path of the centrifuge system, thereby generating an enriched composition having a higher percentage of viable T cells than the percentage of viable T cells in the cell composition, wherein (i) the second centrifugal force is between about 1,500 G and about 3,000 G; (ii) the second flow rate is between about 65 mL/min and about 100 mL/min; and (iii) the ratio of the second centrifugal force (in G) to the second flow rate (in mL/min) is between about 30 and about 40; wherein the T cells have a mean diameter of less than 9 μm.

In some embodiments, the method comprises collecting the elutriated waste fraction. In some embodiments, the elutriated waste fraction is collected in a container that is in fluid communication with the wide end of the conical fluid enclosure.

Also provided herein is a method for enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells, and (b) applying a second centrifugal force and a second flow rate to the cell composition, wherein the second centrifugal force and second flow rate recirculate cells of the cell composition in a fluid path of the centrifuge system, thereby elutriating out of the conical fluid enclosure a waste fraction of the cell composition that has a higher percentage of nonviable T cells than the percentage of nonviable T cells in the cell composition and generating within the conical fluid enclosure an enriched composition that has a higher percentage of viable T cells than the percentage of viable T cells in the cell composition, wherein the cell composition comprises T cells that were cryopreserved and thawed before application of the method. In some embodiments, the centrifuge system is a continuous counterflow centrifuge system.

Also provided herein is a method for enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells, (b) applying a second centrifugal force and a second flow rate to the cell composition, wherein the second centrifugal force and second flow rate recirculate cells of the cell composition in a fluid path of the centrifuge system, thereby elutriating out of the conical fluid enclosure a waste fraction of the cell composition that has a higher percentage of nonviable T cells than the percentage of nonviable T cells in the cell composition and generating within the conical fluid enclosure an enriched composition that has a higher percentage of viable T cells than the percentage of viable T cells in the cell composition, and (c) cryopreserving cells of the enriched composition to create a cryopreserved cell composition following steps (a) and (b). In some embodiments, the method comprises (d) thawing the cryopreserved cell composition following step (c). In some embodiments, the centrifuge system is a continuous counterflow centrifuge system.

Also provided herein is a method of enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells; and (b) applying a second centrifugal force and a second flow rate to the cell composition, wherein the second centrifugal force and second flow rate recirculate cells of the cell composition in a fluid path of the centrifuge system, thereby generating an enriched composition having a higher percentage of viable T cells than the percentage of viable T cells in the cell composition. In some embodiments, the centrifuge system is a continuous counterflow centrifuge system.

In some embodiments, the method comprises collecting the elutriated waste fraction. In some embodiments, the elutriated waste fraction is collected in a container that is in fluid communication with the wide end of the conical fluid enclosure.

In some embodiments, the second flow rate is 30 mL/min or less. In some embodiments, the second flow rate is between about 25 mL/min and about 30 mL/min.

Also provided herein is a method of enriching a cell composition for viable cells, the method comprising: (a) applying a first centrifugal force and a first flow rate to a cell composition comprising T cells in a conical fluid enclosure of a centrifuge system to produce a fluidized bed of cells, wherein the cell composition comprises viable and non-viable T cells, wherein (i) the first centrifugal force is between about 2,000 G and about 4,000 G; and (ii) the first flow rate is between about 5 mL/min and about 15 mL/min; and (b) applying a second centrifugal force and a second flow rate to the cell composition, wherein the second centrifugal force and second flow rate recirculate cells of the cell composition in a fluid path of the centrifuge system, thereby generating an enriched composition having a higher percentage of viable T cells than the percentage of viable T cells in the cell composition, wherein (i) the second centrifugal force is between about 500 G and about 1,500 G; and (ii) the second flow rate is between about 25 mL/min and about 30 mL/min.

In some embodiments, the cell composition comprises T cells that were cryopreserved and thawed before application of the method. In some embodiments, the method further comprises thawing a cryopreserved cell composition to produce the cell composition comprising T cells.

In some embodiments, the second centrifugal force is between about 700 G and about 1,300 G. In some embodiments, the second centrifugal force is between about 800 G and about 1,200 G. In some embodiments, the second centrifugal force is between about 900 G and about 1,100 G. In some embodiments, the second centrifugal force is about 1,000 G.

In some embodiments, the ratio of the second centrifugal force (in G) to the second flow rate (in mL/min) is between about 30 and about 40.

In some embodiments, (i) the first centrifugal force is between about 2,000 G and about 4,000 G; and (ii) the first flow rate is between about 5 mL/min and about 15 mL/min. In some embodiments, (i) the second centrifugal force is between about 500 G and about 1,500 G; and (ii) the second flow rate is between about 25 mL/min and about 30 mL/min. In some embodiments, the method comprises loading the cell composition into the centrifuge system. In some embodiments, the loading is performed prior to and/or during at least a portion of the applying in (a). In some embodiments, the loading is performed prior to the applying in (a). In some embodiments, the loading is performed during at least a portion of the applying in (a). In some embodiments, the loading is performed prior to and during at least a portion of the applying in (a).

In some embodiments, prior to the applying in (a), the method comprises contacting the T cells of the cell composition with a viral vector particle, thereby producing genetically engineered T cells. In some embodiments, the T cells of the cell composition have been contacted with a viral vector particle, thereby producing genetically engineered T cells. In some embodiments, the T cells of the cell composition are genetically engineered T cells.

In some embodiments, the percentage of viable T cells in the enriched composition is at least about 10% greater, at least about 20% greater, at least about 30% greater, at least about 40% greater, at least about 50% greater, or at least about 60% greater than the percentage of viable T cells in the cell composition.

In some embodiments, the method comprises (c) applying a third centrifugal force and a third flow rate to the enriched composition in the conical fluid enclosure of the centrifuge system to collect the enriched composition. In some embodiments, (i) the third centrifugal force is between about 2,000 G and about 3,000 G; and (ii) the third flow rate is between about 15 mL/min and about 25 mL/min. In some embodiments, prior to the applying the third centrifugal force and the third flow rate, the method comprises subjecting the enriched composition to one or more washing steps. In some embodiments, the one or more washing steps comprise media exchange. In some embodiments, the one or more washing steps are carried out at the second centrifugal force and the second flow rate.

In some embodiments, the percentage of viable T cells in the collected enriched composition about 1 day after, about 2 days after, about 3 days after, about 4 days after, about 5 days after, about 6 days after, about 7 days after, about 8 days after, about 9 days after, or about 10 days after collection is greater than the percentage of viable T cells in the cell composition. In some embodiments, the percentage of viable T cells in the collected enriched composition about 1 day after collection is greater than the percentage of viable T cells in the cell composition. In some embodiments, the percentage of viable T cells in the collected enriched composition about 5 days after collection is greater than the percentage of viable T cells in the cell composition.

In some embodiments, the method comprises incubating the T cells of the cell composition under stimulating conditions prior to the applying in (a). In some embodiments, the T cells of the cell compositions are incubated under stimulating conditions prior to the applying in (a). In some embodiments, the cell composition comprises activated cells. In some embodiments, the stimulating conditions comprise the presence of a stimulatory reagent that is capable of activating one or more intracellular signaling domains of one or more components of a TCR complex and one or more intracellular signaling domains of one or more costimulatory molecules. In some embodiments, the stimulatory reagent comprises (i) a primary agent that specifically binds to a member of a TCR complex, optionally that specifically binds to CD3; and (ii) a secondary agent that specifically binds to a T cell costimulatory molecule. In some embodiments, the primary agent specifically binds to CD3. In some embodiments, the secondary agent specifically binds to a costimulatory molecule selected from CD28, CD137 (4-1-BB), OX40, and ICOS. In some embodiments, the secondary agent specifically binds to CD28.

In some embodiments, at least one of the primary and secondary agents comprises an antibody or an antigen-binding fragment thereof. In some embodiments, the primary agent and the secondary agent each comprises an antibody or an antigen-binding fragment thereof. In some embodiments, the primary agent is an anti-CD3 antibody or an antigen-binding fragment thereof. In some embodiments, the secondary agent is an anti-CD28 antibody or an antigen-binding fragment thereof. In some embodiments, the primary agent is an anti-CD3 antibody or an antigen-binding fragment thereof and the secondary agent is an anti-CD28 antibody or an antigen-binding fragment thereof. In some embodiments, the primary agent and the secondary agent are each present or attached on the surface of a solid support. In some embodiments, the solid support is or comprises a bead. In some embodiments, the solid support is a paramagnetic bead with surface attached anti-CD3 and anti-CD28 antibodies. In some embodiments, the primary agent and the secondary agent are reversibly bound on the surface of an oligomeric particle reagent comprising a plurality of streptavidin molecules or streptavidin mutein molecules. In some embodiments, the streptavidin molecules or the streptavidin mutein molecules bind to or are capable of binding to biotin, avidin, a biotin analog or a biotin mutein, an avidin analog or an avidin mutein and/or a biologically active fragment thereof. In some embodiments, the primary agent comprises an anti-CD3 Fab and the secondary agent comprises an anti-CD28 Fab.

In some embodiments, the stimulating conditions comprise the presence of one or more recombinant cytokines. In some embodiments, the stimulating conditions comprise the presence of one or more of recombinant IL-2, IL-7, and IL-15. In some embodiments, the cell composition comprises activated T cells.

In some embodiments, the method comprises cryopreserving the collected enriched composition, thereby generating a cryopreserved enriched composition. In some embodiments, the collected enriched composition is cryopreserved, thereby generating a cryopreserved enriched composition. In some embodiments, the cryopreserved enriched composition is thawed to produce a thawed enriched composition, and the percentage of viable T cells in the thawed enriched composition is greater than the percentage of viable T cells in the cell composition. In some embodiments, the percentage of viable T cells in the thawed enriched composition is at least about 5% greater, at least about 10% greater, at least about 15% greater, at least about 20% greater, at least about 25% greater, or at least about 30% greater than the percentage of viable T cells in the cell composition.

In some embodiments, the method comprises cryopreserving cells of the enriched composition following application of steps (a) and (b) to create a cryopreserved cell composition. In some embodiments, the cryopreserving comprises suspending the cells in a medium comprising a cryoprotectant and freezing the cells. In some embodiments, the freezing is in a controlled rate freezer.

In some embodiments, the method further comprises thawing the cryopreserved cell composition. In some embodiments, the thawing is done after the cryopreserved cell composition has been frozen for at least 3 days.

In some embodiments, one or more steps of the method are automated. In some embodiments, the one or more steps of the method are automated by the centrifuge system or a component thereof.

In some embodiments, the viral vector particle comprises a heterologous nucleic acid encoding a recombinant molecule. In some embodiments, the recombinant molecule is a chemokine, a chemokine receptor, a cytokine, a cytokine receptor, an antigen receptor (e.g., a CAR or a TCR), or a combination thereof. In some embodiments, the recombinant molecule is an antigen receptor. In some embodiments, the antigen receptor is a transgenic T cell receptor (TCR). In some embodiments, is a chimeric antigen receptor (CAR).

In some embodiments, the chimeric antigen receptor (CAR) comprises an extracellular antigen-recognition domain that specifically binds to a target antigen and an intracellular signaling domain comprising an immunoreceptor tyrosine-based activation motif (ITAM). In some embodiments, the intracellular signaling domain comprises an intracellular domain of a CD3-zeta (CD35) chain. In some embodiments, the CAR further comprises a transmembrane domain linking the extracellular domain and the intracellular signaling domain. In some embodiments, the transmembrane domain comprises a transmembrane portion of CD28. In some embodiments, the intracellular signaling domain further comprises an intracellular signaling domain of a T cell costimulatory molecule. In some embodiments, the T cell costimulatory molecule is selected from the group consisting of CD28 and 4-1BB. In some embodiments, the CAR is recombinantly expressed. In some embodiments, the CAR is expressed from a vector. In some embodiments, the CAR is expression from a γ-retroviral vector or a lentiviral vector. In some embodiments, the CAR is expressed from a lentiviral vector.

In some embodiments, the viral vector particle is a retroviral vector particle. In some embodiments, the retroviral vector particle is a γ-retroviral vector. In some embodiments, the retroviral vector particle is a lentiviral vector particle.

In some embodiments, the antigen receptor specifically binds to an antigen associated with a disease or a condition. In some embodiments, the disease or condition is a cancer, an autoimmune disease or disorder, and/or an infectious disease. In some embodiments, the disease or condition is a cancer. In some embodiments, the T cells are primary T cells, optionally from a human subject.

Provided herein is a composition comprising genetically engineered T cells produced by any of the methods provided herein. In some embodiments, the composition comprises between about 1.0×10⁶ CAR-expressing T cells and 2.0×10⁹ CAR-expressing T cells. In some embodiments, the composition comprises a pharmaceutically acceptable carrier. In some embodiments, the composition comprises a cryoprotectant.

Provided herein is a method of treating a subject having a disease or disorder, the method comprising administering any of the compositions provided herein to the subject. Provided herein is use of any of the compositions provided herein for the treatment of a disease or disorder in a subject. Provided herein is any of the compositions provided herein for use in treating a disease or disorder in a subject. In some embodiments, the genetically engineered cells are engineered to express an antigen receptor that specifically binds to an antigen associated with the disease or disorder.

### BRIEF DESCRIPTION OF THE DRAWINGS

FIGS. 1A and 1B show the results of three different experiments enriching input cell compositions for viable cells via continuous counterflow centrifugation (Runs 1-3). FIG. 1A shows the number of total live and dead cells of the input composition, as well as the product and waste fractions following continuous counterflow centrifugation. FIG. 1B shows the increase in the percentage of viable cells following enrichment by continuous counterflow centrifugation.

FIGS. 2A and 2B show the transduction efficiency in the transduced product fraction and the percentage of viable cells in input compositions and various product fractions, respectively, following transduction by viral vector, carried out by either a continuous counterflow centrifugation-based method or a scaled-down spinoculation method. As a control, cells were incubated with the viral vector by the scaled-down method, but not subjected to any centrifugation (“no spin”).

FIG. 3 shows the percentage of viable cells in input compositions and various product fractions, following transduction by viral vector, carried out by either a continuous counterflow centrifugation-based method or a scaled-down spinoculation method. The input compositions subjected to the continuous counterflow centrifugation-based method of transduction comprised either 30 mL or 60 mL of total volume.

FIG. 4A shows the percentage of viable cells in input compositions and washed product fractions generated from 11 different human donors, following either the continuous counterflow elutriation (CCE)-based method of enrichment and buffer exchange (“washing”), or an alternative method of dead-end centrifugation and buffer exchange.

FIG. 4B shows the percentage of viable cells in the pre-wash product (“harvested product”; HP) fraction, the washed product (WP) fraction, the formulated drug product (FDP) fraction, and the cryopreserved drug product (CDP) fraction using the CCE-based method or the alternative method.

FIG. 4C shows the viability of cells in the washed product (WP) fraction, the formulated drug product (FDP) fraction, and the cryopreserved drug product (CDP) fraction, relative to the viability of the pre-wash product (HP) fraction.

FIG. 4D shows the total viable and non-viable cells in the pre-wash product (HP) fraction, the washed product (WP) fraction, and the washed waste fraction.

FIG. 4E shows the viability ratio of the thawed cryopreserved drug product (CDP) fraction to pre-wash product (HP) fraction at various centrifugal forces.

FIG. 4F shows the final product yield using either the modified CCE-based method or the alternative method.

FIG. 4G shows the average viability of thawed cryopreserved drug product (CDP) fractions using the modified CCE-based method (Mod CCE) or the alternative (Alt) method with low, medium, or high cell loads.

FIGS. 4H, 4I, and 4J show the predicted improvement in cell viability of CDPs produced by the modified CCE-based method compared to the alternative method, including among donors with pre-wash product (HP) fractions exhibiting lower viability (FIG. 4H) and donors with low viability using the alternative method (FIG. 4J).

FIG. 4K shows the theoretical and measured concentration of an impurity in incoming media, and the theoretical concentration of the same impurity in outgoing media, with increasing wash volume of the modified CCE-based method.

FIG. 5A shows the number of total and non-viable cells in input compositions, and product and waste fractions following a continuous counterflow centrifugation method of enrichment. The washing step of the enrichment method was carried out at a centrifugal force to flow rate ratio (G/FR) of either 62.5 or 33.3.

FIG. 5B shows the percentage of viable cells in the input composition and the washed product fraction following a washing step performed at a centrifugal force to flow rate ratio (G/FR) of either 62.5 or 33.3.

FIG. 5C shows the size of viable (V) and non-viable (NV) cells in input compositions for viability enrichment that had been stimulated for 96 hours (top panel) or expanded in culture for 15 days after initiation of activation (bottom panel).

FIG. 6A shows the percentage of CD3+CAR+ cells following a continuous counterflow centrifugation-based method of transduction with a viral vector, carried out under different centrifugal forces and flow rates. For comparison, the percentage of CD3+CAR+ cells was assessed following a scaled-down spinoculation method of transduction (FIG. 6B).

FIG. 6C shows the percentage of CD3+CAR+ cells following a continuous counterflow centrifugation-based method of transduction with a viral vector, carried out under conditions with 3000 G centrifugal force and 30 mL/min flow rate. FIG. 6D shows the percentage of CD3+CAR+ cells following a continuous counterflow centrifugation-based method of transduction with a viral vector, carried out under conditions in which centrifugal force and flow rate conditions were periodically varied throughout incubation. For comparison, cells were transduced with the viral vector using a scaled-down spinoculation method (693G).

FIG. 7A shows the percentage of CD3+CAR+ cells among live CD45+ cells, following continuous counterflow centrifugation based-transduction of cells with either 1.11 μL or 3.33 μL of vector particles per million cells.

FIG. 7B shows the percentage of CD3+CAR+ cells following continuous counterflow centrifugation based-transduction of cells with 6 μL of vector particles per million cells. For comparison, cells were transduced with the viral vector using a scaled-down spinoculation method (693G).

FIG. 8A shows flow cytometric analysis of CD3+CAR+ cells following a continuous counterflow centrifugation-based method of transduction, where the input composition comprised either 30 mL or 60 mL of total volume. The results are quantified in FIG. 8B.

FIG. 9A shows flow cytometric analysis of CD3+CAR+ cells following a continuous counterflow centrifugation-based method of transduction, where the input composition comprised either 600×10⁶ total cells or 200×10⁶ total cells. As a control, 15×10⁶ total cells were subjected to a scaled-down spinoculation method of transduction. The results are quantified in FIG. 9B.

FIG. 10A shows the percentage of CAR+ Jurkat cells following transduction with viral vector supernatant taken from a reverse centrifuge system during continuous counterflow centrifugation-based transduction of primary T cells.

FIG. 10B shows the percentage of CD3+CAR+ primary T cells following 30 minutes or 90 minutes of continuous counterflow centrifugation-based transduction in a reverse centrifuge system.

FIG. 11 shows viral vector concentration and distribution in a continuous counterflow centrifuge system during a continuous counterflow centrifugation-based method of transduction.

### DETAILED DESCRIPTION

Provided herein is a method of transducing cells by subjecting a target cell, such as comprised in a cell composition, and a viral vector particle, to centrifugation. In some embodiments, the method of centrifugation is based on continuous counterflow elutriation (CCE). In some embodiments, the method relates to CCE-based centrifugation under conditions in which a target cell is repeatedly contacted by viral vector particles in an enclosure (e.g., a conical enclosure) of a centrifuge, thereby generating a composition comprising a plurality of the target cells transduced with the viral vector. In some embodiments, the centrifugation-based methods of transduction also enrich for viable target cells, remove impurities, or both. Also provided are related compositions containing the transduced cell populations, such as produced by the methods in accord with the provided disclosure.

Also provided are methods of transfer of viral vectors into cells (e.g., T cells) that involve transduction of cells, such as immune cells, e.g., T cells, by centrifugation. In some embodiments, the provided methods involve subjecting cells, such as immune cells (e.g., T cells), and viral vector particles, such as a lentiviral vector, to centrifugation. In some embodiments, the method of centrifugation is or is based on a continuous counterflow elutriation (CCE) method of centrifugation, such as is carried out in a reverse centrifuge system (e.g., a counterflow centrifugation system). In some embodiments, the centrifugation system is any of those described in WO 2018/204992 and WO 2019/140491, each incorporated by reference herein in its entirety. In some embodiments, the provided methods involve subjecting cells, such as immune cells (e.g., T cells), and viral vector particles, such as a lentiviral vector, to centrifugation in a reverse centrifugation system (e.g., a counterflow centrifugation system), wherein the viral vector particles are circulated through the system to repeatedly contact the cells.

Also provided are methods of enriching for viable cells in a cell composition, such as a transduced cell composition, by centrifugation. In some embodiments, the cell composition is a T cell composition, e.g., transduced T cell composition. In some embodiments, the method of centrifugation is or is based on a CCE method of centrifugation, such as is carried out in a reverse centrifuge system (e.g., a counterflow centrifugation system). Also provided are related compositions containing the cell populations enriched for viable cells, such as produced by the methods in accord with the provided disclosure.

Also provided are methods of removing beads from (debeading) a cell composition, e.g., T cell composition, by centrifugation. In some embodiments, the method of centrifugation is or is based on a CCE method of centrifugation, such as is carried out in a reverse centrifuge system (e.g., a counterflow centrifugation system). Also provided are related compositions containing the debeaded cell populations, such as produced by the methods in accord with the provided disclosure.

Reverse flow (also known as “counterflow”) centrifugation is a technique whereby the settling rate of particles in a fluid under centrifugal acceleration is counteracted by a flow of the supporting media. The particles are thereby suspended as a fluidized bed. Reverse flow centrifugation is gentle enough that cells can be cultured, expanding in the fluidized bed state. Cell aggregation can also be reduced. Further, this technique enables separation of dead cell from live cells due to different density and morphology characteristics. Delivering a fluid flow radial inwards to cells or particles under centrifugal acceleration creates a counterflow situation. The centrifugal acceleration experienced by each particle is proportional to the radial distance of that particle from the center of rotation. To create a bed of fluidized particles, the counteracting flow rate needs to be adjusted for each radius of rotation. In any of the provided embodiments, the flow rate is provided by a peristaltic pump. This is achieved by shaping the chamber, commonly as a cone with the tip of the cone pointing radially outwards. The counter fluid flow is input through the cone tip. The fluid flow enters the tip of the cone at a relatively high velocity and the velocity of the fluid flow progressively reduces as it progresses radially inwards due to the increasing cross section of the cone. Historically, there has been investigation into chamber shapes and specific cone geometries for reverse flow centrifugation, as documented in the article by R. J. Sanderson, K. E. Bird, N. F. Palmer and J. Brenman: “Design Principles for a Counter Flow Centrifugation Cell Separation Chamber” Analytical Biochemistry 71, 615-622 (1976).

In some embodiments, the provided methods are used to genetically engineer such cells with a heterologous molecule encoding a recombinant antigen receptor, such as a chimeric antigen receptor (CAR) or transgenic T cell receptor (TCR). The resulting genetically engineered cells can be used in adoptive immunotherapy. In some such embodiments, the provided methods can be used to prepare immune cells, such as T cells, for adoptive therapy, without spinoculation-based methods of transduction. In some aspects, the provided methods enrich a cell composition, a cell and viral vector particle composition, and/or a transduced cell population for viable cells. In some aspects, the enrichment in viable cells is more pronounced in compositions having low starting viability. In some aspects, the provided methods enrich for viable cells while maintaining comparable final cell yield, as compared to an alternative method (e.g., a dead-end centrifugation method).

In general, spinoculation-based methods can be used to transduce cells, such as immune cells (e.g., T cells) with viral vector particles. However, such methods can suffer from low numbers of viable cells and/or low numbers of viable transduced cells, as spinoculation-based methods of transduction do not inherently enrich a cell composition for viable cells. Thus, with existing spinoculation-based methods, it may not always be possible to transduce a large number of cells with maintained viability for downstream use, e.g., for use in cell therapy.

Further, by virtue of a continuous counterflow centrifugation system diluting impurities at a continuous exponential rate, the clearance of impurities in a cellular composition is improved, as compared to alternative methods and systems. In some aspects, impurities can include proteins, DNA, cell debris, reagent(s) used in manufacture, or any combination thereof.

In addition, alternative methods and systems for centrifugation cannot necessarily be automated and/or integrated into other systems. By contrast, the different steps of the methods provided herein can be automated using a continuous counterflow centrifugation system (e.g., the CTS Rotea™ Counterflow Centrifugation System). Further, such systems are compatible and can be integrated with other instruments and systems.

The provided methods are based on observations that continuous counterflow elutriation-based methods of centrifugation, such as is carried out in a reverse centrifuge system (e.g., e.g., the CTS Rotea™ Counterflow Centrifugation System), can achieve sufficient transduction of primary cells obtained from a subject and can enrich the cell composition for viable cells.

In some embodiments, the methods are capable of achieving at least a particular number or percentage of viable cells. For example, in some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% of the cells in the composition generated by the method are viable. In some embodiments, the methods produce an output composition having a greater percentage of viable cells than the input composition, for example, 5%, 10%, 15%, or 20% greater than the input composition.

In some such embodiments, the number or percentage of viable cells can be monitored and/or observed by measuring the level of expression of a marker indicative of cell viability or lack thereof. A number of well-known methods for assessing cell viability may be used, such as detection of cell death markers such as chromatin condensation, Annexin V, caspases, propidium iodide (PI), and/or phosphatidylserine (PS), e.g., in the context of cell surface proteins, such as by flow cytometry or cell staining (e.g., immunohistochemistry). In some examples, the expression is measured by detection of a level of a molecule produced by a cell, such as lactate dehydrogenase (LDH) or adenosinetriphosphate (ATP).

In some embodiments, the methods can be used to transduce a population of T cells in which at least 20%, 30%, 40%, 50%, 60%, 70%, 80%, 90% or more of the T cells in the population are viable T cells, such as T cells that lack a cell death marker, such as a surface marker or secreted molecule or other marker.

In some embodiments, the methods produce an output composition in which at least 25%, at least 30%, at least 40%, at least 50%, or at least 75% of the total cells (or of a particular target cell type, such as T cells) in the output composition, are viable and/or do not express a cell death marker, such as a surface marker or secreted molecule or other marker.

In some embodiments, the provided methods result in a relatively high viability of immune cells, for example T cells. In some embodiments, at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of the cells, such as T cells, in a cell population, e.g., output composition, are viable in accord with the provided methods.

In some embodiments, no more than 5%, 10%, 15%, 20%, 25%, 30%, 35%, 40%, 45%, 50%, 55%, or 60% of the T cells in the output composition are non-viable cells, express a surface marker selected from the group consisting of Annexin V, caspases, propidium iodide (PI) and/or phosphatidylserine (PS); and/or secrete relatively high levels of LDH and/or relatively low levels of ATP. For example, in some aspects, the population of cells of the input composition and/or output composition is one in which at least 40%, 50%, 55%, 60%, 65%, 70%, 75%, 80%, 85%, 90%, 95% are surface negative for Annexin V, caspases, propidium iodide (PI), and/or phosphatidylserine (PS).

Methods and techniques for assessing the viability of cells are known in the art. Antibodies and reagents for detection of such markers are well known in the art, and readily available.

In some embodiments, the methods are capable of achieving at least a particular transduction efficiency under certain conditions. For example, in some embodiments, where the input composition includes the virus and cells at a ratio of from or from about 1 infectious unit (IU) per one of the cells to 10 IU per one of the cells, such as at least or at or about 1 infectious units (IU) per one of the cells, or at least or at or about 2 IU per one of the cells, at least or at or about 5 IU per one of the cells, or at least or at or about 10 IU per one of the cells, the method is capable of producing an output composition in which at least 10%, at least 15%, at least 20%, at least 25%, at least 30%, at least 35%, at least 40%, at least 45%, at least 50%, at least 55%, at least 60%, at least 65%, at least 70%, or at least 75% of the cells in the composition generated by the method comprise, e.g., have been transduced with, the viral vector.

In some such embodiments, the transduction efficiency of cells with a viral vector particle (e.g., retroviral vector) can be monitored and/or observed by measuring the level of expression of a recombinant molecule or protein, such as a heterologous antigen receptor, encoded by a nucleic acid contained in the genome of the viral vector following transduction or other form of transfer of the vector into a cell, such as a viable host cell, such a viable T cell, or population of cells thereof. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the provided methods can include a cryopreservation step prior to or following the incubation, e.g., transduction, of cells with the viral particles. In some embodiments, the provided methods include a cryopreservation step following the incubation, e.g., transduction, of cells with the viral particles. In some embodiments, such a step could provide a break step in the process to allow for shipment of materials, sampling of materials, or ‘hold’ on therapy pending the patient's condition. In some embodiments, the enrichment in cell viability achieved by the provided methods is maintained in a cryopreserved drug product or in a thawed drug product that was previously cryopreserved. In some embodiments, the enrichment in cell viability achieved by the provided methods is maintained in a cryopreserved drug product. In some embodiments, the enrichment in cell viability achieved by the provided methods is maintained in a thawed drug product that was previously cryopreserved.

In some embodiments, the provided methods are carried out such that one, more, or all steps in the preparation of cells for clinical use, e.g., in adoptive cell therapy, are carried out without exposing the cells to non-sterile conditions and without the need to use a sterile room or cabinet. In some embodiments of such a process, the cells are enriched for viability and/or transduced, all within a closed system. In some embodiments, the closed system is or includes a conical enclosure of a reverse centrifuge system. In some embodiments, the methods, or any portion thereof, are carried out in an automated fashion. In some embodiments, the entire method is carried out in an automated fashion.

In some embodiments, the provided methods provide for an optimized or improved process where the cells are transduced and/or enriched for cell viability, in the absence of spinoculation, improving downstream processing and product. In some aspects, the provided methods also may produce transduced cells for administration to a subject with a better or more desirable phenotype, e.g., more viable cells.

In some embodiments, such cells produced by the method, or a composition comprising such cells, are administered to a subject for treating a disease or condition.

In some embodiments, the provided methods include administering to a subject a sub-optimal dose of cells. In some embodiments, the dose of cells is less than or less than about 1.5-fold, 2-fold, 3-fold, 4-fold, 5-fold or 10-fold less than a therapeutically effective dose of cells for treating the disease or condition. In such an example, expansion of cells to yield a therapeutically effective amount of cells can occur in vivo upon administration of cells to a subject.

In some embodiments, the provided methods include in vivo expansion of cells. In some aspects, in vivo expansion of cells can occur in vivo by transgene-specific activation or stimulation of the administered cells. In some embodiment, the antigen receptor (e.g., CAR) is stimulated, such as is activated or expanded, upon recognition of antigen. In some embodiments, one or more agent is administered to the subject to boost, augment, or increase the stimulation, activation, or expansion of the cells in vivo in the subject.

In some embodiments, the provided methods produce genetically engineered T cells that, when administered to a subject, exhibit increased persistence and/or expansion. In some embodiments, a genetically engineered cell with increased persistence and/or expansion exhibits better potency in a subject to which it is administered. In some embodiments, the provided viral vector particles and methods reduce variability in treatment outcomes in adoptive immunotherapy methods, for example, by minimizing and/or reducing the ex vivo manipulations of T cells prior to administration to a subject. In some embodiments, enriching for viable T cells prior to or concurrently with transduction improves the process of producing or preparing genetically engineered T-cells for adoptive immunotherapy by reducing time and reagents required for ex vivo manipulations. In some embodiments, selection for the transduced or engineered cells is carried out following genetic engineering.

Also among the provided embodiments are genetically engineered cells produced by any of the methods provided herein, such as cells expressing a recombinant antigen receptor (e.g., a TCR or a CAR), and methods and uses of such genetically engineered cells for adoptive immunotherapy.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. METHODS OF TRANSDUCING CELLS

Provided are methods for producing a composition of genetically engineered T cells in a centrifuge system. In some embodiments, the methods include transducing an input composition comprising a cell composition with a viral vector particle in a centrifuge system. In some embodiments, the centrifuge system is a continuous counterflow elutriation (“CCE”) centrifuge system, also known as a reverse centrifuge system. In some embodiments, the produced cells are for use in cell therapy, such as primary cells prepared for autologous or allogeneic transfer, e.g., in adoptive cell therapy. The methods may include additional cell processing steps, such as cell washing, isolation, separation, collection, formulation, or other steps related to producing a cell composition.

In some embodiments, the provided methods are used to introduce a viral vector particle, such as a retroviral vector particle, into cells, such as T cells. In some embodiments, the viral vector particles contain a heterologous polynucleotide encoding an antigen receptor, such as a chimeric antigen receptor (CAR) or a transgenic T cell receptor (TCR). Hence, in some embodiments, the provided methods can be used for expressing in cells, such as T cells, a genetically engineered antigen receptor, such as a transgenic TCR or a CAR. Also provided are cells transduced by such particles and methods and compositions containing such cells, and methods for using the same.

In some embodiments, the methods include features that result in an increased viability of transduced cells and/or certain populations or subpopulations thereof, desirable for use in adoptive immunotherapy. In some embodiments, the provided methods enrich for viable cells, including viable genetically engineered cells. Thus, in some embodiments, compositions of cells resulting from the provided methods exhibit increased viability following transduction, as compared to prior to transduction. In some embodiments, the increased viability is observed immediately following transduction and/or is maintained for a duration of time following transduction (e.g., hours or days).

### A. Input Composition

In some embodiments, the provided methods involve genetically engineering T cells by contacting a cell composition comprising T cells with a viral vector particle containing a heterologous polynucleotide encoding an antigen receptor (together an “input composition”) in a centrifuge system. In some embodiments, the method involves applying a first centrifugal force and a first flow rate to a cell composition comprising T cells in a conical fluid enclosure of a centrifuge system to produce a fluidized bed of cells. In some embodiments, the method includes loading the cell composition into the conical fluid enclosure prior to applying the first centrifugal force and the first flow rate.

In some embodiments, the centrifuge system includes a cannula within the conical fluid enclosure. In some embodiments, the cannula runs along the length of the conical fluid enclosure. In some embodiments, one end of the cannula is at or near the tip of the conical fluid enclosure. In some embodiments, the other end of the cannula is at or near the wide end of the conical fluid enclosure, such as at or near the center of the wide end.

In some embodiments, the cell composition is loaded into the conical fluid enclosure via the cannula. In some embodiments, the cell composition is loaded into the conical fluid enclosure at or near the tip of the conical fluid enclosure. In some embodiments, the cell composition is loaded into the conical fluid enclosure by entering the end of the cannula at or near the wide end of the conical fluid enclosure and exiting the end of the cannula at or near the tip of the conical fluid enclosure.

In some embodiments, the method includes loading the viral vector particle into the conical fluid enclosure of the centrifuge system, wherein the resulting composition comprising the cell composition and the viral vector particle is an input composition. In some embodiments, the loading of the viral vector particle is carried out concurrently with or during the applying of the first centrifugal force and the first flow rate.

In some embodiments, the viral vector particle is loaded into the conical fluid enclosure via the cannula. In some embodiments, the viral vector particle is loaded into the conical fluid enclosure at or near the tip of the conical fluid enclosure. In some embodiments, the viral vector particle is loaded into the conical fluid enclosure by entering the end of the cannula at or near the wide end of the conical fluid enclosure and exiting the end of the cannula at or near the tip of the conical fluid enclosure.

In some embodiments, the method includes applying a second centrifugal force and a second flow rate to the input composition. In some embodiments, the second centrifugal force and second flow rate recirculate the viral vector particle in a fluid path of the centrifuge system. Thus, in some embodiments, the viral vector particle repeatedly contacts the fluidized bed of cells during recirculation through the centrifuge system. In some embodiments, the methods produce genetically engineered T cells expressing the antigen receptor.

In some embodiments, applying the second centrifugal force and second flow rate thereby elutriates out of the conical fluid enclosure a waste fraction of the cell composition. In some embodiments, the elutriated waste fraction has a higher percentage of nonviable T cells than the percentage of nonviable T cells in the cell composition.

In some embodiments, the elutriated cells exit the conical fluid enclosure via an opening at the wide end of the conical fluid enclosure. In some embodiments, the opening at least partially surrounds the end of the cannula at or near the wide end of the conical fluid enclosure. In some embodiments, the opening surrounds the end of the cannula at or near the wide end of the conical fluid enclosure.

In some embodiments, the method involves collecting the elutriated waste fraction. In some embodiments, the elutriated waste fraction is collected in a container. In some embodiments, the container is in fluid communication with the wide end of the conical fluid enclosure.

In some embodiments, applying the second centrifugal force and second flow rate thereby produces within the conical fluid enclosure the enriched composition that has a higher percentage of viable T cells than the percentage of viable T cells in the cell composition.

In some embodiments, the loading of the viral vector particle is carried out concurrently with or during the applying of the second centrifugal force and the second flow rate. In some embodiments, the viral vector particle is present in the centrifuge system during at least a portion of the applying of the second centrifugal force and the second flow rate.

In some embodiments, the methods include (a) applying a first centrifugal force and a first flow rate to a cell composition comprising T cells in a conical fluid enclosure of a centrifuge system to produce a fluidized bed of cells; (b) loading a viral vector particle containing a heterologous polynucleotide encoding an antigen receptor into the conical fluid enclosure, thereby generating an input composition comprising the cell composition and the viral vector particle; and (c) applying a second centrifugal force and a second flow rate to the input composition, wherein the second centrifugal force and second flow rate recirculate the viral vector particle in a fluid path of the centrifuge system such that the viral vector particle repeatedly contacts the fluidized bed of cells, thereby generating genetically engineered T cells.

In some embodiments, the method involves applying a first centrifugal force and a first flow rate to an input composition comprising a viral vector particle containing a heterologous polynucleotide encoding an antigen receptor and a cell composition comprising T cells. In some embodiments, the viral vector particle and the cell composition are both present in a conical fluid enclosure of a centrifuge system when the first centrifugal force and the first flow rate are applied. In some embodiments, the applying of the first centrifugal force and the first flow rate produces a fluidized bed of cells. In some embodiments, the method comprises loading the cell composition and the viral vector particle into the conical fluid enclosure prior to the applying the first centrifugal force and the first flow rate. In some embodiments, the loading of the cell composition is before, during, and/or after loading of the viral vector particle. In some embodiments, the method comprises loading the input composition into the conical fluid enclosure prior to the applying the first centrifugal force and the first flow rate. In some embodiments, the cell composition and/or the viral vector particle are loaded into the conical fluid enclosure prior to or during the applying the first centrifugal force and the first flow rate.

In some embodiments, the first centrifugal force is between about 625 G and about 3,000 G. In some embodiments, the first centrifugal force is between about 625 G and about 3,000 G, between about 625 G and about 2,500 G, between about 625 G and about 2,000 G, between about 625 G and about 1,500 G, between about 625 G and about 1,000 G, between about 1,000 G and about 3,000 G, between about 1,000 G and about 2,500 G, between about 1,000 G and about 2,000 G, between about 1,000 G and about 1,500 G, between about 1,500 G and about 3,000 G, between about 1,500 G and about 2,500 G, between about 1,500 G and about 2,000 G, between about 2,000 G and about 3,000 G, between about 2,000 G and about 2,500 G, or between about 2,500 G and about 3,000 G. In some embodiments, the first centrifugal force is between about 2,000 G and about 4,000 G.

In some embodiments, the first centrifugal force is between about 1,000 G and about 5,000 G, between about 1,500 G and about 4,500 G, between about 2,000 G and about 4,000 G, between about 1,500 G and about 3,500 G, or between about 2,000 G and about 3,000 G. In some embodiments, the first centrifugal force is about 1,000 G. In some embodiments, the first centrifugal force is about 1,500 G. In some embodiments, the first centrifugal force is about 2,000 G. In some embodiments, the first centrifugal force is about 2,500 G. In some embodiments, the first centrifugal force is about 3,000 G. In some embodiments, the first centrifugal force is about 3,500 G. In some embodiments, the first centrifugal force is about 4,000 G. In some embodiments, the first centrifugal force is about 4,500 G. In some embodiments, the first centrifugal force is about 5,000 G.

In some embodiments, the first flow rate is radially inward. In some embodiments, the first flow rate is directed away from the tip of the conical fluid enclosure. In some embodiments, the first centrifugal force is counteracted by the first flow rate. In some embodiments, the first flow rate is a counterflow rate.

In some embodiments, the first flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In some embodiments, the first flow rate is between about 1 mL/min and about 20 mL/min, between about 3 mL/min and about 18 mL/min, between about 5 mL/min and about 15 mL/min, or between about 8 mL/min and about 12 mL/min. In some embodiments, the first flow rate is about 1 mL/min. In some embodiments, the first flow rate is about 3 mL/min. In some embodiments, the first flow rate is about 5 mL/min. In some embodiments, the first flow rate is about 8 mL/min. In some embodiments, the first flow rate is about 9 mL/min. In some embodiments, the first flow rate is about 10 mL/min. In some embodiments, the first flow rate is about 11 mL/min. In some embodiments, the first flow rate is about 12 mL/min. In some embodiments, the first flow rate is about 15 mL/min. In some embodiments, the first flow rate is about 18 mL/min. In some embodiments, the first flow rate is about 20 mL/min.

In some embodiments, the first flow rate is between about 1 mL/min and about 50 mL/min, between about 1 mL/min and about 40 mL/min, between about 1 mL/min and about 30 mL/min, between about 1 mL/min and about 20 mL/min, between about 1 mL/min and about 10 mL/min, between about 10 mL/min and about 50 mL/min, between about 10 mL/min and about 40 mL/min, between about 10 mL/min and about 30 mL/min, between about 10 mL/min and about 20 mL/min, between about 20 mL/min and about 50 mL/min, between about 20 mL/min and about 40 mL/min, between about 20 mL/min and about 30 mL/min, between about 30 mL/min and about 50 mL/min, between about 30 mL/min and about 40 mL/min, or between about 40 mL/min and about 50 mL/min. In some embodiments, the first flow rate is between about 10 mL/min and about 40 mL/min.

In some embodiments, the first flow rate is between about 10 mL/min and about 50 mL/min, between about 20 mL/min and about 50 mL/min, between about 30 mL/min and about 50 mL/min, or between about 35 mL/min and about 45 mL/min. In some embodiments, the first flow rate is about 40 mL/min.

In some embodiments, the first flow rate is between about 5 mL/min and about 15 mL/min. In some embodiments, (i) the first centrifugal force is between about 2,000 G and about 4,000 G; and (ii) the first flow rate is between about 5 mL/min and about 15 mL/min.

In some embodiments, the ratio of the first centrifugal force to the first flow rate is between about 200 and about 400. Ratios of centrifugal force to flow rate herein are ratios of centrifugal force in G to flow rate in mL/min, unless otherwise indicated.

In some embodiments, the ratio of the first centrifugal force to the first flow rate is between about 200 and about 400, between about 225 and about 375, between about 250 and about 350, or between about 275 and about 325. In some embodiments, the ratio of the first centrifugal force to the first flow rate is about 200. In some embodiments, the ratio of the first centrifugal force to the first flow rate is about 200. In some embodiments, the ratio of the first centrifugal force to the first flow rate is about 225. In some embodiments, the ratio of the first centrifugal force to the first flow rate is about 250. In some embodiments, the ratio of the first centrifugal force to the first flow rate is about 275. In some embodiments, the ratio of the first centrifugal force to the first flow rate is about 300. In some embodiments, the ratio of the first centrifugal force to the first flow rate is about 325. In some embodiments, the ratio of the first centrifugal force to the first flow rate is about 350. In some embodiments, the ratio of the first centrifugal force to the first flow rate is about 375. In some embodiments, the ratio of the first centrifugal force to the first flow rate is about 400. In some embodiments, the first centrifugal force is about 3,000 G and the first flow rate is about 10 mL/min.

In some embodiments, the ratio of the first centrifugal force to the first flow rate is between about 20 and 200, between about 20 and 180, between about 20 and 160, between about 20 and 140, between about 20 and 120, between about 20 and 100, between about 20 and 80, between about 20 and 60, between about 20 and 40, between about 40 and 200, between about 40 and 180, between about 40 and 160, between about 40 and 140, between about 40 and 120, between about 40 and 100, between about 40 and 80, between about 40 and 60, between about 60 and 200, between about 60 and 180, between about 60 and 160, between about 60 and 140, between about 60 and 120, between about 60 and 100, between about 60 and 80, between about 80 and 200, between about 80 and 180, between about 80 and 160, between about 80 and 140, between about 80 and 120, between about 80 and 100, between about 100 and 200, between about 100 and 180, between about 100 and 160, between about 100 and 140, between about 100 and 120, between about 120 and 200, between about 120 and 180, between about 120 and 160, between about 120 and 140, between about 140 and 200, between about 140 and 180, between about 140 and 160, between about 160 and 200, between about 160 and 180, or between about 180 and 200. In some embodiments, the ratio of the first centrifugal force to the first flow rate is between about 40 and 200, between about 40 and 180, between about 40 and 160, between about 40 and 140, between about 40 and 120, between about 40 and 100, between about 40 and 80, or between about 50 and 60. In some embodiments, the ratio of the first centrifugal force to the first flow rate is about 62.5.

In some embodiments, the ratio of the first centrifugal force to the first flow rate is between about 62.5 and 300, between about 62.5 and 250, between about 62.5 and 200, between about 62.5 and 150, or between about 62.5 and 100. In some embodiments, the ratio of the first centrifugal force to the first flow rate is about 62.5.

In some embodiments, the ratio of the first centrifugal force to the first flow rate is between about 62.5 and 300, between about 100 and 300, between about 150 and 300, between about 200 and 300, and between about 250 and 300.

In some embodiments, the first centrifugal force is about 2500 G and the first flow rate is about 40 mL/min.

In some embodiments, the first centrifugal force and the first flow rate are applied to the cell composition for about 15 seconds, about 30 seconds, about 45 seconds, about 60 seconds, about 2 minutes, about 3 minutes, about 4 minutes, about 5 minutes, about 10 minutes, or about 15 minutes. In some embodiments, the first centrifugal force and the first flow rate are applied to the cell composition for about 15 seconds. In some embodiments, the first centrifugal force and the first flow rate are applied to the cell composition for about 30 seconds. In some embodiments, the first centrifugal force and the first flow rate are applied to the cell composition for about 45 seconds. In some embodiments, the first centrifugal force and the first flow rate are applied to the cell composition for about 60 seconds. In some embodiments, the first centrifugal force and the first flow rate are applied to the cell composition for between about 1 minute and about 15 minutes, between about 3 minutes and about 12 minutes, or between about 5 minutes and about 10 minutes. In some embodiments, the first centrifugal force and the first flow rate are applied to the cell composition for about 1 minute. In some embodiments, the first centrifugal force and the first flow rate are applied to the cell composition for about 2 minutes. In some embodiments, the first centrifugal force and the first flow rate are applied to the cell composition for about 3 minutes. In some embodiments, the first centrifugal force and the first flow rate are applied to the cell composition for about 4 minutes. In some embodiments, the first centrifugal force and the first flow rate are applied to the cell composition for about 5 minutes. In some embodiments, the first centrifugal force and the first flow rate are applied to the cell composition for about 6 minutes. In some embodiments, the first centrifugal force and the first flow rate are applied to the cell composition for about 7 minutes. In some embodiments, the first centrifugal force and the first flow rate are applied to the cell composition for about 8 minutes. In some embodiments, the first centrifugal force and the first flow rate are applied to the cell composition for about 9 minutes. In some embodiments, the first centrifugal force and the first flow rate are applied to the cell composition for about 10 minutes. In some embodiments, the first centrifugal force and the first flow rate are applied to the cell composition for about 11 minutes. In some embodiments, the first centrifugal force and the first flow rate are applied to the cell composition for about 12 minutes. In some embodiments, the first centrifugal force and the first flow rate are applied to the cell composition for about 13 minutes. In some embodiments, the first centrifugal force and the first flow rate are applied to the cell composition for about 14 minutes. In some embodiments, the first centrifugal force and the first flow rate are applied to the cell composition for about 15 minutes. In some embodiments, the first centrifugal force and the first flow rate are applied to the cell composition at least until a fluidized bed of cells is established.

In some embodiments, the first centrifugal force and the first flow rate are applied to the cell composition for at least about 15 seconds, at least about 30 seconds, at least about 45 seconds, at least about 60 seconds, at least about 2 minutes, at least about 3 minutes, at least about 4 minutes, at least about 5 minutes, at least about 10 minutes, or at least about 15 minutes. In some embodiments, the first centrifugal force and the first flow rate are applied to the cell composition for at least about 15 seconds. In some embodiments, the first centrifugal force and the first flow rate are applied to the cell composition for at least about 20 seconds. In some embodiments, the first centrifugal force and the first flow rate are applied to the cell composition for at least about 25 seconds. In some embodiments, the first centrifugal force and the first flow rate are applied to the cell composition for at least about 30 seconds. In some embodiments, the first centrifugal force and the first flow rate are applied to the cell composition for at least about 45 seconds. In some embodiments, the first centrifugal force and the first flow rate are applied to the cell composition for at least about 60 seconds. In some embodiments, the first centrifugal force and the first flow rate are applied to the cell composition for at least about 2 minutes. In some embodiments, the first centrifugal force and the first flow rate are applied to the cell composition for at least about 3 minutes. In some embodiments, the first centrifugal force and the first flow rate are applied to the cell composition for at least about 4 minutes. In some embodiments, the first centrifugal force and the first flow rate are applied to the cell composition for at least about 5 minutes. In some embodiments, the first centrifugal force and the first flow rate are applied to the cell composition for at least about 10 minutes. In some embodiments, the first centrifugal force and the first flow rate are applied to the cell composition for at least about 15 minutes.

In some embodiments, the first centrifugal force and the first flow rate are applied to the cell composition for between or between about 15 seconds and 60 seconds. In some embodiments, the first centrifugal force and the first flow rate are applied to the cell composition for between or between about 25 seconds and 60 seconds. In some embodiments, the first centrifugal force and the first flow rate are applied to the cell composition for between or between about 30 seconds and 60 seconds. In some embodiments, the first centrifugal force and the first flow rate are applied to the cell composition for between or between about 15 seconds and 2 minutes. In some embodiments, the first centrifugal force and the first flow rate are applied to the cell composition for between or between about 25 seconds and 2 minutes.

In some embodiments, the first centrifugal force and the first flow rate are applied to the cell composition until a predetermined number of cells are loaded into the conical fluid enclosure. In some embodiments, the first centrifugal force and the first flow rate are applied to the cell composition until a predetermined number of cells are part of the fluidized bed of cells. In some embodiments, the predetermined number of cells is a predetermined number of T cells. In some embodiments, the predetermined number of cells, e.g., T cells, is between about 10 million and 100 million, between about 10 million and 90 million, between about 10 million and 80 million, between about 10 million and 70 million, between about 10 million and 60 million, between about 10 million and 50 million, between about 10 million and 40 million, between about 10 million and 30 million, between about 10 million and 20 million, between about 20 million and 100 million, between about 20 million and 90 million, between about 20 million and 80 million, between about 20 million and 70 million, between about 20 million and 60 million, between about 20 million and 50 million, between about 20 million and 40 million, between about 20 million and 30 million, between about 30 million and 100 million, between about 30 million and 90 million, between about 30 million and 80 million, between about 30 million and 70 million, between about 30 million and 60 million, between about 30 million and 50 million, between about 30 million and 40 million, between about 40 million and 100 million, between about 40 million and 90 million, between about 40 million and 80 million, between about 40 million and 70 million, between about 40 million and 60 million, between about 40 million and 50 million, between about 50 million and 100 million, between about 50 million and 90 million, between about 50 million and 80 million, between about 50 million and 70 million, between about 50 million and 60 million, between about 60 million and 100 million, between about 60 million and 90 million, between about 60 million and 80 million, between about 60 million and 70 million, between about 70 million and 100 million, between about 70 million and 90 million, between about 70 million and 80 million, between about 80 million and 100 million, between about 80 million and 90 million, or between about 90 million and 100 million cells, e.g., T cells. In some embodiments, the predetermined number of cells, e.g., T cells, is about 50 million cells, e.g., T cells.

In some embodiments, the first centrifugal force and the first flow rate are applied to the input composition for about 15 seconds, about 30 seconds, about 45 seconds, about 60 seconds, about 2 minutes, about 3 minutes, about 4 minutes, about 5 minutes, about 10 minutes, or about 15 minutes. In some embodiments, the first centrifugal force and the first flow rate are applied to the input composition for about 15 seconds. In some embodiments, the first centrifugal force and the first flow rate are applied to the input composition for about 30 seconds. In some embodiments, the first centrifugal force and the first flow rate are applied to the input composition for about 45 seconds. In some embodiments, the first centrifugal force and the first flow rate are applied to the input composition for about 60 seconds. In some embodiments, the first centrifugal force and the first flow rate are applied to the input composition for between about 1 minute and about 15 minutes, between about 3 minutes and about 12 minutes, or between about 5 minutes and about 10 minutes. In some embodiments, the first centrifugal force and the first flow rate are applied to the input composition for about 1 minute. In some embodiments, the first centrifugal force and the first flow rate are applied to the input composition for about 2 minutes. In some embodiments, the first centrifugal force and the first flow rate are applied to the input composition for about 3 minutes. In some embodiments, the first centrifugal force and the first flow rate are applied to the input composition for about 4 minutes. In some embodiments, the first centrifugal force and the first flow rate are applied to the input composition for about 5 minutes. In some embodiments, the first centrifugal force and the first flow rate are applied to the input composition for about 6 minutes. In some embodiments, the first centrifugal force and the first flow rate are applied to the input composition for about 7 minutes. In some embodiments, the first centrifugal force and the first flow rate are applied to the input composition for about 8 minutes. In some embodiments, the first centrifugal force and the first flow rate are applied to the input composition for about 9 minutes. In some embodiments, the first centrifugal force and the first flow rate are applied to the input composition for about 10 minutes. In some embodiments, the first centrifugal force and the first flow rate are applied to the input composition for about 11 minutes. In some embodiments, the first centrifugal force and the first flow rate are applied to the input composition for about 12 minutes. In some embodiments, the first centrifugal force and the first flow rate are applied to the input composition for about 13 minutes. In some embodiments, the first centrifugal force and the first flow rate are applied to the input composition for about 14 minutes. In some embodiments, the first centrifugal force and the first flow rate are applied to the input composition for about 15 minutes. In some embodiments, the first centrifugal force and the first flow rate are applied to the input composition at least until a fluidized bed of cells is established.

In some embodiments, the first centrifugal force and the first flow rate are applied to the input composition for at least about 15 seconds, at least about 30 seconds, at least about 45 seconds, at least about 60 seconds, at least about 2 minutes, at least about 3 minutes, at least about 4 minutes, at least about 5 minutes, at least about 10 minutes, or at least about 15 minutes. In some embodiments, the first centrifugal force and the first flow rate are applied to the input composition for at least about 15 seconds. In some embodiments, the first centrifugal force and the first flow rate are applied to the input composition for at least about 20 seconds. In some embodiments, the first centrifugal force and the first flow rate are applied to the input composition for at least about 25 seconds. In some embodiments, the first centrifugal force and the first flow rate are applied to the input composition for at least about 30 seconds. In some embodiments, the first centrifugal force and the first flow rate are applied to the input composition for at least about 45 seconds. In some embodiments, the first centrifugal force and the first flow rate are applied to the input composition for at least about 60 seconds. In some embodiments, the first centrifugal force and the first flow rate are applied to the input composition for at least about 2 minutes. In some embodiments, the first centrifugal force and the first flow rate are applied to the input composition for at least about 3 minutes. In some embodiments, the first centrifugal force and the first flow rate are applied to the input composition for at least about 4 minutes. In some embodiments, the first centrifugal force and the first flow rate are applied to the input composition for at least about 5 minutes. In some embodiments, the first centrifugal force and the first flow rate are applied to the input composition for at least about 10 minutes. In some embodiments, the first centrifugal force and the first flow rate are applied to the input composition for at least about 15 minutes.

In some embodiments, the first centrifugal force and the first flow rate are applied to the input composition for between or between about 15 seconds and 60 seconds. In some embodiments, the first centrifugal force and the first flow rate are applied to the input composition for between or between about 25 seconds and 60 seconds. In some embodiments, the first centrifugal force and the first flow rate are applied to the input composition for between or between about 30 seconds and 60 seconds. In some embodiments, the first centrifugal force and the first flow rate are applied to the input composition for between or between about 15 seconds and 2 minutes. In some embodiments, the first centrifugal force and the first flow rate are applied to the input composition for between or between about 25 seconds and 2 minutes.

In some embodiments, the first centrifugal force and the first flow rate are applied to the input composition until a predetermined number of cells are loaded into the conical fluid enclosure. In some embodiments, the first centrifugal force and the first flow rate are applied to the input composition until a predetermined number of cells are part of the fluidized bed of cells. In some embodiments, the predetermined number of cells is a predetermined number of T cells. In some embodiments, the predetermined number of cells, e.g., T cells, is between about 10 million and 100 million, between about 10 million and 90 million, between about 10 million and 80 million, between about 10 million and 70 million, between about 10 million and 60 million, between about 10 million and 50 million, between about 10 million and 40 million, between about 10 million and 30 million, between about 10 million and 20 million, between about 20 million and 100 million, between about 20 million and 90 million, between about 20 million and 80 million, between about 20 million and 70 million, between about 20 million and 60 million, between about 20 million and 50 million, between about 20 million and 40 million, between about 20 million and 30 million, between about 30 million and 100 million, between about 30 million and 90 million, between about 30 million and 80 million, between about 30 million and 70 million, between about 30 million and 60 million, between about 30 million and 50 million, between about 30 million and 40 million, between about 40 million and 100 million, between about 40 million and 90 million, between about 40 million and 80 million, between about 40 million and 70 million, between about 40 million and 60 million, between about 40 million and 50 million, between about 50 million and 100 million, between about 50 million and 90 million, between about 50 million and 80 million, between about 50 million and 70 million, between about 50 million and 60 million, between about 60 million and 100 million, between about 60 million and 90 million, between about 60 million and 80 million, between about 60 million and 70 million, between about 70 million and 100 million, between about 70 million and 90 million, between about 70 million and 80 million, between about 80 million and 100 million, between about 80 million and 90 million, or between about 90 million and 100 million cells, e.g., T cells. In some embodiments, the predetermined number of cells, e.g., T cells, is about 50 million cells, e.g., T cells.

In some embodiments, the second flow rate is radially inward. In some embodiments, the second flow rate is directed away from the tip of the conical fluid enclosure. In some embodiments, the second centrifugal force is counteracted by the second flow rate. In some embodiments, the second flow rate is a counterflow rate.

In some embodiments, the second flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In other embodiments, the second flow rate is radially outward. In some embodiments, the second flow rate is directed toward the tip of the conical fluid enclosure. In some embodiments, the second centrifugal force and the second flow rate are in the same or substantially the same direction.

In some embodiments, the second flow rate is effected by the flow of media through the conical fluid enclosure. In some embodiments, the flow of media through the conical fluid enclosure is from the wide end to the tip of the conical fluid enclosure. In some embodiments, the media exits the tip of the conical fluid enclosure to enter the cannula.

In some embodiments, the second centrifugal force is between about 100 G and about 2,000 G, between about 200 G and about 1800 G, between about 500 G and about 1,500 G, or between about 750 G and about 1,250 G. In some embodiments, the second centrifugal force is about 250 G. In some embodiments, the second centrifugal force is about 500 G. In some embodiments, the second centrifugal force is about 600 G. In some embodiments, the second centrifugal force is about 700 G. In some embodiments, the second centrifugal force is about 800 G. In some embodiments, the second centrifugal force is about 900 G. In some embodiments, the second centrifugal force is about 1,000 G. In some embodiments, the second centrifugal force is about 1,100 G. In some embodiments, the second centrifugal force is about 1,200 G. In some embodiments, the second centrifugal force is about 1,300 G. In some embodiments, the second centrifugal force is about 1,400 G. In some embodiments, the second centrifugal force is about 1,500 G. In some embodiments, the second centrifugal force is about 1,300 G. In some embodiments, the second centrifugal force is about 1,750 G. In some embodiments, the second centrifugal force is about 2,000 G.

In some embodiments, the second centrifugal force is between about 100 G and about 4,000 G, between about 100 G and about 3,750 G, between about 100 G and about 3,500 G, between about 100 G and about 3,250 G, between about 100 G and about 3,000 G, between about 100 G and about 2,750 G, between about 100 G and about 2,500 G, between about 100 G and about 2,250 G, between about 100 G and about 2,000 G, between about 100 G and about 1,750 G, between about 100 G and about 1,500 G, between about 100 G and about 1,250 G, between about 100 G and about 1,000 G, between about 100 G and about 750 G, between about 100 G and about 500 G, between about 100 G and about 250 G, between about 250 G and about 4,000 G, between about 250 G and about 3,750 G, between about 250 G and about 3,500 G, between about 250 G and about 3,250 G, between about 250 G and about 3,000 G, between about 250 G and about 2,750 G, between about 250 G and about 2,500 G, between about 250 G and about 2,250 G, between about 250 G and about 2,000 G, between about 250 G and about 1,750 G, between about 250 G and about 1,500 G, between about 250 G and about 1,250 G, between about 250 G and about 1,000 G, between about 250 G and about 750 G, between about 250 G and about 500 G, between about 500 G and about 4,000 G, between about 500 G and about 3,750 G, between about 500 G and about 3,500 G, between about 500 G and about 3,250 G, between about 500 G and about 3,000 G, between about 500 G and about 2,750 G, between about 500 G and about 2,500 G, between about 500 G and about 2,250 G, between about 500 G and about 2,000 G, between about 500 G and about 1,750 G, between about 500 G and about 1,500 G, between about 500 G and about 1,250 G, between about 500 G and about 1,000 G, between about 500 G and about 750 G, between about 750 G and about 4,000 G, between about 750 G and about 3,750 G, between about 750 G and about 3,500 G, between about 750 G and about 3,250 G, between about 750 G and about 3,000 G, between about 750 G and about 2,750 G, between about 750 G and about 2,500 G, between about 750 G and about 2,250 G, between about 750 G and about 2,000 G, between about 750 G and about 1,750 G, between about 750 G and about 1,500 G, between about 750 G and about 1,250 G, between about 750 G and about 1,000 G, between about 1,000 G and about 4,000 G, between about 1,000 G and about 3,750 G, between about 1,000 G and about 3,500 G, between about 1,000 G and about 3,250 G, between about 1,000 G and about 3,000 G, between about 1,000 G and about 2,750 G, between about 1,000 G and about 2,500 G, between about 1,000 G and about 2,250 G, between about 1,000 G and about 2,000 G, between about 1,000 G and about 1,750 G, between about 1,000 G and about 1,500 G, between about 1,000 G and about 1,250 G, between about 1,250 G and about 4,000 G, between about 1,250 G and about 3,750 G, between about 1,250 G and about 3,500 G, between about 1,250 G and about 3,250 G, between about 1,250 G and about 3,000 G, between about 1,250 G and about 2,750 G, between about 1,250 G and about 2,500 G, between about 1,250 G and about 2,250 G, between about 1,250 G and about 2,000 G, between about 1,250 G and about 1,750 G, between about 1,250 G and about 1,500 G, between about 1,500 G and about 4,000 G, between about 1,500 G and about 3,750 G, between about 1,500 G and about 3,500 G, between about 1,500 G and about 3,250 G, between about 1,500 G and about 3,000 G, between about 1,500 G and about 2,750 G, between about 1,500 G and about 2,500 G, between about 1,500 G and about 2,250 G, between about 1,500 G and about 2,000 G, between about 1,500 G and about 1,750 G, between about 1,750 G and about 4,000 G, between about 1,750 G and about 3,750 G, between about 1,750 G and about 3,500 G, between about 1,750 G and about 3,250 G, between about 1,750 G and about 3,000 G, between about 1,750 G and about 2,750 G, between about 1,750 G and about 2,500 G, between about 1,750 G and about 2,250 G, between about 1,750 G and about 2,000 G, between about 2,000 G and about 4,000 G, between about 2,000 G and about 3,750 G, between about 2,000 G and about 3,500 G, between about 2,000 G and about 3,250 G, between about 2,000 G and about 3,000 G, between about 2,000 G and about 2,750 G, between about 2,000 G and about 2,500 G, between about 2,000 G and about 2,250 G, between about 2,250 G and about 4,000 G, between about 2,250 G and about 3,750 G, between about 2,250 G and about 3,500 G, between about 2,250 G and about 3,250 G, between about 2,250 G and about 3,000 G, between about 2,250 G and about 2,750 G, between about 2,250 G and about 2,500 G, between about 2,500 G and about 4,000 G, between about 2,500 G and about 3,750 G, between about 2,500 G and about 3,500 G, between about 2,500 G and about 3,250 G, between about 2,500 G and about 3,000 G, between about 2,500 G and about 2,750 G, between about 2,750 G and about 4,000 G, between about 2,750 G and about 3,750 G, between about 2,750 G and about 3,500 G, between about 2,750 G and about 3,250 G, between about 2,750 G and about 3,000 G, between about 3,000 G and about 4,000 G, between about 3,000 G and about 3,750 G, between about 3,000 G and about 3,500 G, between about 3,000 G and about 3,250 G, between about 3,250 G and about 4,000 G, between about 3,250 G and about 3,750 G, between about 3,250 G and about 3,500 G, between about 3,500 G and about 4,000 G, between about 3,500 G and about 3,750 G, or between about 3,750 G and about 4,000 G.

In some embodiments, the second centrifugal force is between about 500 G and about 1,500 G. In some embodiments, the second centrifugal force is between about 500 G and about 1,500 G, between about 500 G and about 1,400 G, between about 500 G and about 1,300 G, between about 500 G and about 1,200 G, between about 500 G and about 1,100 G, between about 500 G and about 1,000 G, between about 500 G and about 900 G, between about 500 G and about 800 G, or between about 500 G and about 700 G. In some embodiments, the second centrifugal force is about 625 G.

In some embodiments, the second centrifugal force is between about 100 G and about 2,000 G. In some embodiments, the second centrifugal force is between about 100 G and about 2,000 G, between about 100 G and about 1,900 G, between about 100 G and about 1,800 G, between about 100 G and about 1,700 G, between about 100 G and about 1,600 G, between about 100 G and about 1,500 G, between about 100 G and about 1,400 G, between about 100 G and about 1,300 G, between about 100 G and about 1,200 G, between about 100 G and about 1,100 G, between about 100 G and about 1,000 G, between about 100 G and about 900 G, between about 100 G and about 800 G, between about 100 G and about 700 G, between about 100 G and about 600 G, between about 100 G and about 500 G, or between about 100 G and about 400 G. In some embodiments, the second centrifugal force is about 300 G.

In some embodiments, the second flow rate is between about 10 and about 100 mL/min. In some embodiments, (i) the second centrifugal force is between about 100 G and about 2,000 G; and (ii) the second flow rate is between about 10 mL/min and about 100 mL/min. In some embodiments, (i) the second centrifugal force is between about 500 G and about 1,500 G; and (ii) the second flow rate is between about 10 mL/min and about 100 mL/min.

In some embodiments, the second flow rate is between about 10 and about 100 mL/min, between about 15 and about 90 mL/min, between about 20 and about 80 mL/min, between about 25 and about 70 mL/min, between about 30 and about 60 mL/min, or between about 35 and about 50 mL/min. In some embodiments, the second flow rate is about 20 mL/min, about 21 mL/min, about 22 mL/min, about 23 mL/min, about 24 mL/min, about 25 mL/min, about 25.5 mL/min, about 26 mL/min, about 26.5 mL/min, about 27 mL/min, about 27.5 mL/min, about 28 mL/min, about 28.5 mL/min, about 29 mL/min, about 29.5 mL/min, about 30 mL/min, about 31 mL/min, about 32 mL/min, about 33 mL/min, about 34 mL/min, or about 35 mL/min. In some embodiments, the second flow rate is about 25 mL/min. In some embodiments, the second flow rate is about 25.5 mL/min. In some embodiments, the second flow rate is about 26 mL/min. In some embodiments, the second flow rate is about 26.5 mL/min. In some embodiments, the second flow rate is about 27 mL/min. In some embodiments, the second flow rate is about 27.5 mL/min. In some embodiments, the second flow rate is about 28 mL/min. In some embodiments, the second flow rate is about 28.5 mL/min. In some embodiments, the second flow rate is about 29 mL/min. In some embodiments, the second flow rate is about 29.5 mL/min. In some embodiments, the second flow rate is about 30 mL/min.

In some embodiments, the second flow rate is between about 5 and about 100 mL/min, between about 5 and about 90 mL/min, between about 5 and about 80 mL/min, between about 5 and about 70 mL/min, between about 5 and about 60 mL/min, between about 5 and about 50 mL/min, between about 5 and about 40 mL/min, between about 5 and about 30 mL/min, between about 5 and about 25 mL/min, between about 5 and about 20 mL/min, between about 5 and about 15 mL/min, between about 5 and about 10 mL/min, between about 10 and about 100 mL/min, between about 10 and about 90 mL/min, between about 10 and about 80 mL/min, between about 10 and about 70 mL/min, between about 10 and about 60 mL/min, between about 10 and about 50 mL/min, between about 10 and about 40 mL/min, between about 10 and about 30 mL/min, between about 10 and about 25 mL/min, between about 10 and about 20 mL/min, between about 10 and about 15 mL/min, between about 15 and about 100 mL/min, between about 15 and about 90 mL/min, between about 15 and about 80 mL/min, between about 15 and about 70 mL/min, between about 15 and about 60 mL/min, between about 15 and about 50 mL/min, between about 15 and about 40 mL/min, between about 15 and about 30 mL/min, between about 15 and about 25 mL/min, between about 15 and about 20 mL/min, between about 20 and about 100 mL/min, between about 20 and about 90 mL/min, between about 20 and about 80 mL/min, between about 20 and about 70 mL/min, between about 20 and about 60 mL/min, between about 20 and about 50 mL/min, between about 20 and about 40 mL/min, between about 20 and about 30 mL/min, between about 20 and about 25 mL/min, between about 25 and about 100 mL/min, between about 25 and about 90 mL/min, between about 25 and about 80 mL/min, between about 25 and about 70 mL/min, between about 25 and about 60 mL/min, between about 25 and about 50 mL/min, between about 25 and about 40 mL/min, between about 25 and about 30 mL/min, between about 30 and about 100 mL/min, between about 30 and about 90 mL/min, between about 30 and about 80 mL/min, between about 30 and about 70 mL/min, between about 30 and about 60 mL/min, between about 30 and about 50 mL/min, between about 30 and about 40 mL/min, between about 40 and about 100 mL/min, between about 40 and about 90 mL/min, between about 40 and about 80 mL/min, between about 40 and about 70 mL/min, between about 40 and about 60 mL/min, between about 40 and about 50 mL/min, between about 50 and about 100 mL/min, between about 50 and about 90 mL/min, between about 50 and about 80 mL/min, between about 50 and about 70 mL/min, between about 50 and about 60 mL/min, between about 60 and about 100 mL/min, between about 60 and about 90 mL/min, between about 60 and about 80 mL/min, between about 60 and about 70 mL/min, between about 70 and about 100 mL/min, between about 70 and about 90 mL/min, between about 70 and about 80 mL/min, between about 80 and about 100 mL/min, between about 80 and about 90 mL/min, or between about 90 and about 100 mL/min. In some embodiments, the second flow rate is between about 10 mL/min and about 30 mL/min. In some embodiments, the second flow rate is between about 25 mL/min and about 30 mL/min.

In some embodiments, (i) the second centrifugal force is between about 500 G and about 1,500 G; and (ii) the second flow rate is between about 25 mL/min and about 30 mL/min.

In some embodiments, the second flow rate is between about 5 and about 40 mL/min, between about 5 and about 35 mL/min, between about 5 and about 30 mL/min, between about 5 and about 25 mL/min, between about 5 and about 20 mL/min, or between about 5 and about 15 mL/min. In some embodiments, the second flow rate is about 10 mL/min.

In some embodiments, the ratio of the second centrifugal force to the second flow rate is between about 20 and about 100. In some embodiments, the ratio of the second centrifugal force to the second flow rate is between about 25 and about 85. In some embodiments, the ratio of the second centrifugal force to the second flow rate is between about 30 and about 65.

In some embodiments, the ratio of the second centrifugal force to the second flow rate is between about 20 and about 100, between about 25 and about 80, or between about 30 and about 60. In some embodiments, the ratio of the second centrifugal force to the second flow rate is about 20. In some embodiments, the ratio of the second centrifugal force to the second flow rate is about 25. In some embodiments, the ratio of the second centrifugal force to the second flow rate is about 30. In some embodiments, the ratio of the second centrifugal force to the second flow rate is about 35. In some embodiments, the ratio of the second centrifugal force to the second flow rate is about 40. In some embodiments, the ratio of the second centrifugal force to the second flow rate is about 45. In some embodiments, the ratio of the second centrifugal force to the second flow rate is about 50. In some embodiments, the ratio of the second centrifugal force to the second flow rate is about 55. In some embodiments, the ratio of the second centrifugal force to the second flow rate is about 60. In some embodiments, the ratio of the second centrifugal force to the second flow rate is about 65. In some embodiments, the ratio of the second centrifugal force to the second flow rate is about 70. In some embodiments, the ratio of the second centrifugal force to the second flow rate is about 75. In some embodiments, the second centrifugal force is about 1,000 G and the second flow rate is about 28.5 mL/min.

In some embodiments, the ratio of the second centrifugal force to the second flow rate is between about 20 and 200, between about 20 and 180, between about 20 and 160, between about 20 and 140, between about 20 and 120, between about 20 and 100, between about 20 and 80, between about 20 and 60, between about 20 and 40, between about 40 and 200, between about 40 and 180, between about 40 and 160, between about 40 and 140, between about 40 and 120, between about 40 and 100, between about 40 and 80, between about 40 and 60, between about 60 and 200, between about 60 and 180, between about 60 and 160, between about 60 and 140, between about 60 and 120, between about 60 and 100, between about 60 and 80, between about 80 and 200, between about 80 and 180, between about 80 and 160, between about 80 and 140, between about 80 and 120, between about 80 and 100, between about 100 and 200, between about 100 and 180, between about 100 and 160, between about 100 and 140, between about 100 and 120, between about 120 and 200, between about 120 and 180, between about 120 and 160, between about 120 and 140, between about 140 and 200, between about 140 and 180, between about 140 and 160, between about 160 and 200, between about 160 and 180, or between about 180 and 200.

In some embodiments, the ratio of the second centrifugal force to the second flow rate is between about 20 and 100, between about 20 and 80, between about 20 and 60, or between about 20 and 40. In some embodiments, the ratio of the second centrifugal force to the second flow rate is about 30. In some embodiments, the second centrifugal force is about 300 G and the second flow rate is about 10 mL/min.

In some embodiments, the ratio of the second centrifugal force to the second flow rate is between about 40 and 200, between about 40 and 180, between about 40 and 160, between about 40 and 140, between about 40 and 120, between about 40 and 100, between about 40 and 80, or between about 50 and 60. In some embodiments, the ratio of the second centrifugal force to the second flow rate is about 62.5. In some embodiments, the second centrifugal force is about 625 G and the second flow rate is about 10 mL/min.

In some embodiments, the second centrifugal force and the second flow rate are applied to the input composition for between about 5 minutes and about 100 minutes, between about 10 minutes and about 90 minutes, between about 15 minutes and about 80 minutes, between about 20 minutes and about 70 minutes, between about 25 minutes and about 60 minutes, between about 30 minutes and about 50 minutes, or between about 35 minutes and about 40 minutes. In some embodiments, the second centrifugal force and the second flow rate are applied to the input composition for about 5 minutes. In some embodiments, the second centrifugal force and the second flow rate are applied to the input composition for about 10 minutes. In some embodiments, the second centrifugal force and the second flow rate are applied to the input composition for about 15 minutes. In some embodiments, the second centrifugal force and the second flow rate are applied to the input composition for about 20 minutes. In some embodiments, the second centrifugal force and the second flow rate are applied to the input composition for about 25 minutes. In some embodiments, the second centrifugal force and the second flow rate are applied to the input composition for about 30 minutes. In some embodiments, the second centrifugal force and the second flow rate are applied to the input composition for about 45 minutes. In some embodiments, the second centrifugal force and the second flow rate are applied to the input composition for about 60 minutes. In some embodiments, the second centrifugal force and the second flow rate are applied to the input composition for about 75 minutes. In some embodiments, the second centrifugal force and the second flow rate are applied to the input composition for about 90 minutes.

In some embodiments, the second centrifugal force and the second flow rate are applied to the input composition for at least about 15 minutes. In some embodiments, the second centrifugal force and the second flow rate are applied to the input composition for at least about 30 minutes. In some embodiments, the second centrifugal force and the second flow rate are applied to the input composition for at least about 45 minutes. In some embodiments, the second centrifugal force and the second flow rate are applied to the input composition for at least about 60 minutes. In some embodiments, the second centrifugal force and the second flow rate are applied to the input composition for at least about 75 minutes. In some embodiments, the second centrifugal force and the second flow rate are applied to the input composition for at least about 90 minutes.

In some embodiments, the second centrifugal force and the second flow rate are continuously applied to the input composition.

In other embodiments, the second centrifugal force and the second flow rate are non-continuously applied to the input composition. In some embodiments, the second centrifugal force and the second flow rate are intermittently applied to the input composition. In some embodiments, the second centrifugal force and the second flow rate are applied to the input composition at regularly occurring intervals.

In any of the preceding embodiments, the amount of time in which the second centrifugal force and the second flow rate are applied to the input composition can be the total amount of time that the second centrifugal force and the second flow rate are non-continuously applied. For example, in some embodiments, the second centrifugal force and the second flow rate are non-continuously applied to the input composition for a total of between about 5 minutes and about 25 minutes during a 30-minute time period. In some embodiments, the second centrifugal force and the second flow rate are applied to the input composition for a total of about 15 minutes during a 30-minute time period.

In some embodiments, the second centrifugal force and the second flow rate are applied to the input composition in a first time period that is followed by a second time period in which the second centrifugal force and the second flow rate are not applied to the input composition. In some embodiments, a centrifugal force and flow rate that differ from the second centrifugal force and the second flow rate, respectively, are applied to the input composition. In some embodiments, the centrifugal force and flow rate applied during the second time period are any of the centrifugal forces and flow rates described herein, for instance any of the described second centrifugal forces and second flow rates, such as at any of the ratios of second centrifugal force to second flow rate described herein.

In some embodiments, the first time period and the second time period are about equal in length.

In other embodiments, the first time period and the second time period differ in length.

In some embodiments, the first and second time periods are repeated a predetermined number of times. In some embodiments, the first and second time periods are repeated until a predetermined amount of time has elapsed. In some embodiments, the predetermined amount of time is between about 5 minutes and about 100 minutes, between about 10 minutes and about 90 minutes, between about 15 minutes and about 80 minutes, between about 20 minutes and about 70 minutes, between about 25 minutes and about 60 minutes, between about 30 minutes and about 50 minutes, or between about 35 minutes and about 40 minutes.

In some embodiments, the first time period is between about 1 minute and about 10 minutes, between about 1 minute and about 9 minutes, between about 1 minute and about 8 minutes, between about 1 minute and about 7 minutes, between about 1 minute and about 6 minutes, between about 1 minute and about 5 minutes, between about 1 minute and about 4 minutes, between about 1 minute and about 3 minutes, between about 1 minute and about 2 minutes, between about 2 minutes and about 10 minutes, between about 2 minutes and about 9 minutes, between about 2 minutes and about 8 minutes, between about 2 minutes and about 7 minutes, between about 2 minutes and about 6 minutes, between about 2 minutes and about 5 minutes, between about 2 minutes and about 4 minutes, between about 2 minutes and about 3 minutes, between about 3 minutes and about 10 minutes, between about 3 minutes and about 9 minutes, between about 3 minutes and about 8 minutes, between about 3 minutes and about 7 minutes, between about 3 minutes and about 6 minutes, between about 3 minutes and about 5 minutes, between about 3 minutes and about 4 minutes, between about 4 minutes and about 10 minutes, between about 4 minutes and about 9 minutes, between about 4 minutes and about 8 minutes, between about 4 minutes and about 7 minutes, between about 4 minutes and about 6 minutes, between about 4 minutes and about 5 minutes, between about 5 minutes and about 10 minutes, between about 5 minutes and about 9 minutes, between about 5 minutes and about 8 minutes, between about 5 minutes and about 7 minutes, between about 5 minutes and about 6 minutes, between about 6 minutes and about 10 minutes, between about 6 minutes and about 9 minutes, between about 6 minutes and about 8 minutes, between about 6 minutes and about 7 minutes, between about 7 minutes and about 10 minutes, between about 7 minutes and about 9 minutes, between about 7 minutes and about 8 minutes, between about 8 minutes and about 10 minutes, between about 8 minutes and about 9 minutes, or between about 9 minutes and about 10 minutes. In some embodiments, the first time period is between about 1 minute and about 10 minutes, between about 1 minute and about 9 minutes, between about 1 minute and about 8 minutes, between about 1 minute and about 7 minutes, between about 1 minute and about 6 minutes, between about 1 minute and about 5 minutes, between about 1 minute and about 4 minutes, between about 1 minute and about 3 minutes, or between about 1 minute and about 2 minutes. In some embodiments, the first time period is about 1 minute.

In some embodiments, the second time period is between about 1 minute and about 10 minutes, between about 1 minute and about 9 minutes, between about 1 minute and about 8 minutes, between about 1 minute and about 7 minutes, between about 1 minute and about 6 minutes, between about 1 minute and about 5 minutes, between about 1 minute and about 4 minutes, between about 1 minute and about 3 minutes, between about 1 minute and about 2 minutes, between about 2 minutes and about 10 minutes, between about 2 minutes and about 9 minutes, between about 2 minutes and about 8 minutes, between about 2 minutes and about 7 minutes, between about 2 minutes and about 6 minutes, between about 2 minutes and about 5 minutes, between about 2 minutes and about 4 minutes, between about 2 minutes and about 3 minutes, between about 3 minutes and about 10 minutes, between about 3 minutes and about 9 minutes, between about 3 minutes and about 8 minutes, between about 3 minutes and about 7 minutes, between about 3 minutes and about 6 minutes, between about 3 minutes and about 5 minutes, between about 3 minutes and about 4 minutes, between about 4 minutes and about 10 minutes, between about 4 minutes and about 9 minutes, between about 4 minutes and about 8 minutes, between about 4 minutes and about 7 minutes, between about 4 minutes and about 6 minutes, between about 4 minutes and about 5 minutes, between about 5 minutes and about 10 minutes, between about 5 minutes and about 9 minutes, between about 5 minutes and about 8 minutes, between about 5 minutes and about 7 minutes, between about 5 minutes and about 6 minutes, between about 6 minutes and about 10 minutes, between about 6 minutes and about 9 minutes, between about 6 minutes and about 8 minutes, between about 6 minutes and about 7 minutes, between about 7 minutes and about 10 minutes, between about 7 minutes and about 9 minutes, between about 7 minutes and about 8 minutes, between about 8 minutes and about 10 minutes, between about 8 minutes and about 9 minutes, or between about 9 minutes and about 10 minutes. In some embodiments, the second time period is between about 1 minute and about 10 minutes, between about 1 minute and about 9 minutes, between about 1 minute and about 8 minutes, between about 1 minute and about 7 minutes, between about 1 minute and about 6 minutes, between about 1 minute and about 5 minutes, between about 1 minute and about 4 minutes, between about 1 minute and about 3 minutes, or between about 1 minute and about 2 minutes. In some embodiments, the second time period is about 1 minute.

In some embodiments, the second centrifugal force is about 1,500 G, the second flow rate is about 10 mL/min, and the second centrifugal force and the second flow rate are applied to the input composition for a first time period of about 1 minute, after which a centrifugal force of about 300 G and a flow rate of about 10 mL/min are applied to the input composition for a second time period of about 1 minute. In some embodiments, the first and second time period are repeated until about 30 minutes have elapsed.

In some embodiments, the volume of the input composition comprises between about 5 ml and about 20,000 ml, between about 10 mL and about 2,000 mL. between about 15 mL and about 1,000 mL, between about 20 mL and about 500 mL, between about 25 mL and about 100 mL, or between about 30 mL and about 60 mL. In some embodiments, the volume of the input composition is between about 30 mL and about 60 mL. In some embodiments, the volume of the input composition is about 5 ml. In some embodiments, the volume of the input composition is about 10 ml. In some embodiments, the volume of the input composition is about 15 ml. In some embodiments, the volume of the input composition is about 20 ml. In some embodiments, the volume of the input composition is about 25 ml. In some embodiments, the volume of the input composition is about 30 ml. In some embodiments, the volume of the input composition is about 35 ml. In some embodiments, the volume of the input composition is about 40 ml. In some embodiments, the volume of the input composition is about 45 ml. In some embodiments, the volume of the input composition is about 50 ml. In some embodiments, the volume of the input composition is about 55 ml. In some embodiments, the volume of the input composition is about 60 ml. In some embodiments, the volume of the input composition is about 65 ml. In some embodiments, the volume of the input composition is about 70 ml. In some embodiments, the volume of the input composition is about 75 ml.

The term “G” or “relative centrifugal force” (RCF) is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, cells produced from the provided method (hereinafter also called “genetically engineered T cells” or “output composition”) include those transduced with the viral vector, such as a viral vector containing a polynucleotide encoding a heterologous protein, such as a recombinant receptor, e.g., a CAR. By heterologous in this context refers to a protein that is not normally expressed from a virus and/or not encoded by a viral genome. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

i. Cell Composition

In some embodiments, a cell composition comprising T cells is transduced with a viral vector particle, such as in accord with the described methods. In some embodiments, the concentration of cells of the input composition is from or from about 1.0×10⁵ cells/mL to 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the cell composition comprises about 1×10⁶ cells/mL. In some embodiments, the cell composition comprises about 1.25×10⁶ cells/mL. In some embodiments, the cell composition comprises about 1.5×10⁶ cells/mL. In some embodiments, the cell composition comprises about 1.75×10⁶ cells/mL. In some embodiments, the cell composition comprises about 2×10⁶ cells/mL. In some embodiments, the cell composition comprises about 2.25×10⁶ cells/mL. In some embodiments, the cell composition comprises about 2.5×10⁶ cells/mL. In some embodiments, the cell composition comprises about 2.75×10⁶ cells/mL. In some embodiments, the cell composition comprises about 3×10⁶ cells/mL.

In some embodiments, the volume of the cell composition comprises between about 20 mL and about 300 mL, between about 25 mL and about 250 mL, between about 30 mL and about 200 mL, between about 35 mL and between about 150 mL, or between about 40 mL and about 100 mL. In some embodiments, the volume of the cell composition is about 20 mL. In some embodiments, the volume of the cell composition is about 25 mL. In some embodiments, the volume of the cell composition is about 30 mL. In some embodiments, the volume of the cell composition is about 35 mL. In some embodiments, the volume of the cell composition is about 40 mL. In some embodiments, the volume of the cell composition is about 45 mL. In some embodiments, the volume of the cell composition is about 50 mL. In some embodiments, the volume of the cell composition is about 55 mL. In some embodiments, the volume of the cell composition is about 60 mL. In some embodiments, the volume of the cell composition is about 70 mL. In some embodiments, the volume of the cell composition is about 80 mL. In some embodiments, the volume of the cell composition is about 100 mL. In some embodiments, the volume of the cell composition is about 125 mL. In some embodiments, the volume of the cell composition is about 150 mL. In some embodiments, the volume of the cell composition is about 175 mL. In some embodiments, the volume of the cell composition is about 200 mL.

In some embodiments, the cell composition comprises about 1×10⁸ total cells, about 2×10⁸ total cells, about 3×10⁸ total cells, about 4×10⁸ total cells, about 5×10⁸ total cells, about 6×10⁸ total cells, about 7×10⁸ total cells, about 8×10⁸ total cells, about 9×10⁸ total cells, or about 1×10⁹ total cells.

In some embodiments, the T cells have a mean diameter of about 5 μm to about 25 μm, about 5 μm to about 20 μm, about 5 μm to about 15 μm, about 5 μm to about 10 μm, about 10 μm to about 25 μm, about 10 μm to about 20 μm, about 10 μm to about 15 μm, about 15 μm to about 25 μm, about 15 μm to about 20 μm, or about 20 μm to about 25 μm. In some embodiments, the T cells have a mean diameter of about 9 μm to about 20 μm.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm.

In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the cell composition contains T cells that were cryopreserved and thawed before application of the method. In some embodiments, the method involves thawing a cryopreserved cell composition to produce the cell composition comprising T cells.

In some embodiments, the cell composition contains T cells that have not been cryopreserved or thawed before application of the method.

In some embodiments, the cells are incubated and/or cultured prior to transduction in accord with the provided methods. The incubation steps can include culture, cultivation, stimulation, activation, and/or propagation. In some embodiments, the method comprises incubating the T cells of the cell composition under stimulating conditions prior to the applying the first centrifugal force and the first flow rate. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the T cells of the cell composition are incubated under stimulating conditions prior to the applying the first centrifugal force and the first flow rate. In some embodiments, the method comprises incubating the T cells of the cell composition under stimulating conditions prior to the loading the cell composition into the centrifuge system. In some embodiments, the T cells of the cell composition are incubated under stimulating conditions prior to the loading the cell composition into the centrifuge system. In some embodiments, the cell composition comprises activated T cells. In some embodiments, the cell composition comprises T cells expressing HLA-DR, CD25, CD69, CD71, CD40L, 4-1BB, or a combination thereof.

In some embodiments, the stimulating conditions comprise the presence of a stimulatory reagent. In some embodiments, the stimulatory reagent is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. In some embodiments, the stimulatory reagent is capable of activating one or more intracellular signaling domains of one or more components of a TCR complex and one or more intracellular signaling domains of one or more costimulatory molecules. In some embodiments, the stimulatory reagent comprises (i) a primary agent that specifically binds to a member of a TCR complex; and (ii) a secondary agent that specifically binds to a T cell costimulatory molecule. In some embodiments, the primary agent specifically binds to CD3. In some embodiments, the costimulatory the costimulatory molecule is selected from CD28, CD137 (4-1-BB), OX40 or ICOS. In some embodiments, at least one of the primary and secondary agents comprises an antibody or an antigen-binding fragment thereof. In some embodiments, the primary agent is or comprises an anti-CD3 antibody or antigen-binding fragment thereof. In some embodiments, the secondary agent is or comprises an anti-CD28 antibody or antigen-binding fragment thereof. Optionally, the expansion method may further comprise the step of adding anti-CD3 and/or anti CD28 antibody to the culture medium (e.g., at a concentration of at least about 0.5 ng/ml).

In some embodiments, the stimulating agents include IL-2 and/or IL-15, for example, an IL-2 concentration of at least about 10 units/mL. In some aspects, incubation is carried out in accordance with techniques such as those described in U.S. Pat. No. 6,040,177 to Riddell et al., Klebanoff et al. (2012) J Immunother. 35 (9): 651-660, Terakura et al. (2012) Blood. 1:72-82, and/or Wang et al. (2012) J Immunother. 35 (9): 689-701.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some cases, a viral vector particle may be used that does not require that the cells, e.g., T cells, are activated. In some such instances, the cells may be selected and/or transduced prior to activation and or in the absence of activation.

In some embodiments, at least 40%, 50%, 60%, 70%, 80%, 90% or more of the cells, e.g., T cells, in the cell composition are activated, such as, in some cases, are surface positive for one or more of HLA-DR, CD25, CD69, CD71, CD40L and/or 4-1BB. In some embodiments, cells are activated with an activating agent, such as in the presence of anti-CD3/anti-CD28, prior to initiation of the applying the first centrifugal force and the first flow rate, e.g., prior to establishment of the fluidized bed and/or prior to initiation of transduction. Methods of expanding T cell populations in vitro in the absence of exogenous growth factors or low amounts of exogenous growth factors are known in the art (see, e.g., U.S. Pat. No. 6,352,694 B1 and European Patent EP 0 700 430 B1). In general, such methods employ a solid phase surfaces of greater than 1 μM to which various bind agents (e.g., anti-CD3 antibody and/or anti-CD28 antibody) are immobilized. For example, Dynabeads® CD3/CD28 (Invitrogen) are commercially available reagents for T cell expansion, which are uniform, 4.5 μm superparamagnetic, sterile, non-pyrogenic polystyrene beads coated with a mixture of affinity purified monoclonal antibodies against the CD3 and CD28 cell surface molecules on human T cells In some embodiments, the activating agent, e.g., anti-CD3 and/or anti-CD28, can be immobilized on beads, such as magnetic beads.

In some embodiments, the cell activation is also performed in the presence IL-2 (e.g., from or from about 50 IU/mL to 200 IU/mL, such as or about 100 IU/mL). In some embodiments, the activation is carried out between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the activation is carried out at a temperature greater than or greater than about 25° C., such as generally greater than or greater than about 32° C., 35° C. or 37° C., for example at or about 37° C.±2° C., such as at a temperature of at or about 37° C.

In some embodiments, cells are not activated with an activating agent, such as in the presence of anti-CD3/anti-CD28, prior to initiation of the contacting, e.g., prior to initiation of transduction. In some embodiments, the cell composition comprises a plurality of resting cells. In some embodiments, at least 40%, 50%, 60%, 70%, 80%, 90% or more of the T cells in the population are resting T cells, such as T cells that lack a T cell activation marker, such as a surface marker or intracellular cytokine or other marker, and/or T cells that are in the G0 or G1 stage of the cell cycle.

In particular aspects, the provided methods allow transduction to happen in T cells without the need for activation prior to the contacting and/or incubation. In some embodiments, the methods include transducing a population of T cells that contain resting or naïve T cells with a viral vector without first, e.g., prior to the transduction, activating and/or stimulating the T cells. In some such embodiments, the provided methods can be used to prepare cells, such as T cells, for adoptive therapy, that do not include a step of activating and/or stimulating T cells.

In some embodiments, the cells generally are eukaryotic cells, such as mammalian cells, and typically are human cells. In some embodiments, the cells are derived from the blood, bone marrow, lymph, or lymphoid organs, or are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells are primary T cells. In some embodiments, the cells are primary T cells from a human subject. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells comprise or are enriched for CD3+ T cells. In some embodiments, the cells comprise or are enriched for CD4+ T cells. In some embodiments, the cells comprise or are enriched for CD8+ T cells. In some embodiments the cells comprise CD4+ and CD8+ T cells. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, preparation of the cells includes one or more culture and/or preparation steps. The cells may be isolated from a sample, such as a biological sample, e.g., one obtained from or derived from a subject. In some embodiments, the subject from which the cells are isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In some embodiments, the cells are PBMCs. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, it is not necessary to enrich or select cells prior to performing the provided method.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. Separation methods may include any of those disclosed herein, including methods using reversible reagent systems, e.g., agents (such as receptor binding agents or selection agents) and reagents as described herein.

In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some embodiments, the cell composition is enriched for CD3+ T cells. In some embodiments, at least 70%, at least 75%, at least 80%, at least 80%, at least 85%, at least 90%, at least 95%, or at least 99% of the cells in the cell composition are CD3+ T cells. In some embodiments, the cell composition is enriched for CD4+ T cells. In some embodiments, at least 70%, at least 75%, at least 80%, at least 80%, at least 85%, at least 90%, at least 95%, or at least 99% of the cells in the cell composition are CD4+ T cells. In some embodiments, the cell composition is enriched for CD8+ T cells. In some embodiments, at least 70%, at least 75%, at least 80%, at least 80%, at least 85%, at least 90%, at least 95%, or at least 99% of the cells in the cell composition are CD8+ T cells. In some embodiments, the cell composition is enriched for CD4+ and CD8+ T cells. In some embodiments, at least 70%, at least 75%, at least 80%, at least 80%, at least 85%, at least 90%, at least 95%, or at least 99% of the cells in the cell composition are CD4+ or CD8+ T cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakuraet al. (2012) Blood. 1:72-82; Wang et al. (2012) J Immunother. 35 (9): 689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher @ Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead contains a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US20110003380 A1.

In some embodiments, the system or apparatus carries out one or more, e.g., all, of the isolation, processing, and/or formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. In certain embodiments, separation and/or other steps are carried out using the CliniMACS Prodigy system (Miltenyi Biotec). The CliniMACS Prodigy system in some aspects is equipped with a cell processing unit that permits automated washing and fractionation of cells by centrifugation.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5): 355-376. In both cases, cells can be labeled with multiple markers, allowing for the isolation of well-defined T cell subsets at high purity.

In some embodiments, the preparation methods include steps for freezing, e.g., cryopreserving, the cells, either before or after isolation, incubation, and/or genetic engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

ii. Viral Vector Particles

In some embodiments, a cell composition comprising T cells is transduced with a viral vector particle, such as any of those described in Section IV, by any of the methods provided herein.

In some embodiments, the lentiviral vector is loaded into the centrifuge in a composition. In some embodiments, the volume of the composition comprising the lentiviral vector particle is about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL. In some embodiments, the volume of the composition comprises the lentiviral vector is about 5 mL. In some embodiments, the volume of the composition comprises the lentiviral vector is about 10 mL. In some embodiments, the volume of the composition comprises the lentiviral vector is about 15 mL. In some embodiments, the volume of the composition comprises the lentiviral vector is about 20 mL.

In some embodiments, the viral vector particles are provided at a certain ratio of copies of the viral vector particles or infectious units (IU) thereof, per total number of cells (IU/cell) in the input composition or total number of cells to be transduced. For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, transduction can be achieved at a multiplicity of infection (MOI) of less than 100, such as generally less than 60, 50, 40, 30, 20, 10, 5 or less.

### B. Output Composition

In some embodiments, the methods comprise applying a third centrifugal force and a third flow rate to the genetically engineered T cells to produce an output composition comprising the genetically engineered T cells. In some embodiments, the output composition is collected or harvested, such as for downstream use in a cell therapy. In some embodiments, the third centrifugal force and the third flow rate are applied to the genetically engineered T cells in the conical fluid enclosure of the centrifuge system. In some embodiments, the application of the third centrifugal force and the third flow rate to the genetically engineered cells allows for the collection or harvest of the output composition.

In some embodiments, the output composition is collected via the cannula. In some embodiments, the output composition enters the end of the cannula at or near the tip of the conical fluid enclosure and exits the end of the cannula at or near the wide end of the conical fluid enclosure.

In some embodiments, the method involves cryopreserving the output composition. In some embodiments, the level of viable cells in the output composition is maintained in the cryopreserved output composition. In some embodiments, the method involves thawing the cryopreserved output composition. In some embodiments, the level of viable cells in the output composition is maintained in the thawed output composition.

In some embodiments, the third centrifugal force is between about 2,000 G and about 3,000 G.

In some embodiments, the third centrifugal force is between about 2,000 G and about 3,000 G, between about 2,200 G and about 2,800 G, or between about 2,400 G and about 2,600 G. In some embodiments, the third centrifugal force is about 2,000 G. In some embodiments, the third centrifugal force is about 2,100 G. In some embodiments, the third centrifugal force is about 2,200 G. In some embodiments, the third centrifugal force is about 2,300 G. In some embodiments, the third centrifugal force is about 2,400 G. In some embodiments, the third centrifugal force is about 2,500 G. In some embodiments, the third centrifugal force is about 2,600 G. In some embodiments, the third centrifugal force is about 2,700 G. In some embodiments, the third centrifugal force is about 2,800 G. In some embodiments, the third centrifugal force is about 2,900 G. In some embodiments, the third centrifugal force is about 3,000 G.

In some embodiments, the third flow rate is radially outward. In some embodiments, the third flow rate is directed toward the tip of the conical fluid enclosure. In some embodiments, the third centrifugal force and the third flow rate are in the same or substantially the same direction.

In some embodiments, the third flow rate is effected by the flow of media through the conical fluid enclosure. In some embodiments, the flow of media through the conical fluid enclosure is from the wide end to the tip of the conical fluid enclosure. In some embodiments, the media exits the tip of the conical fluid enclosure to enter the cannula.

In some embodiments, the third flow rate is between about 10 mL/min and about 30 mL/min, between about 12 mL/min and about 28 mL/min, between about 15 mL/min and about 25 mL/min, or between about 18 mL/min and about 22 mL/min. In some embodiments, the third flow rate is about 15 mL/min. In some embodiments, the third flow rate is about 16 mL/min. In some embodiments, the third flow rate is about 17 mL/min. In some embodiments, the third flow rate is about 18 mL/min. In some embodiments, the third flow rate is about 19 mL/min. In some embodiments, the third flow rate is about 20 mL/min. In some embodiments, the third flow rate is about 21 mL/min. In some embodiments, the third flow rate is about 22 mL/min. In some embodiments, the third flow rate is about 23 mL/min. In some embodiments, the third flow rate is about 24 mL/min. In some embodiments, the third flow rate is about 15 mL/min. In some embodiments, the third flow rate is about 25 mL/min.

In some embodiments, the third flow rate is between about 15 mL/min and about 25 mL/min. In some embodiments, (i) the third centrifugal force is between about 2,000 G and about 3,000 G; and (ii) the third flow rate is between about 15 mL/min and about 25 mL/min.

In some embodiments, the ratio of the third centrifugal force to the third flow rate is between about 100 and about 150, between about 110 and 140, or between about 120 and 130. In some embodiments, the ratio of the third centrifugal force to the third flow rate is about 100. In some embodiments, the ratio of the third centrifugal force to the third flow rate is about 105. In some embodiments, the ratio of the third centrifugal force to the third flow rate is about 110. In some embodiments, the ratio of the third centrifugal force to the third flow rate is about 115. In some embodiments, the ratio of the third centrifugal force to the third flow rate is about 120. In some embodiments, the ratio of the third centrifugal force to the third flow rate is about 125. In some embodiments, the ratio of the third centrifugal force to the third flow rate is about 130. In some embodiments, the ratio of the third centrifugal force to the third flow rate is about 135. In some embodiments, the ratio of the third centrifugal force to the third flow rate is about 140. In some embodiments, the ratio of the third centrifugal force to the third flow rate is about 145. In some embodiments, the ratio of the third centrifugal force to the third flow rate is about 150. In some embodiments, the third centrifugal force is about 2,500 G and the third flow rate is about 20 mL/min.

In some embodiments, the ratio of the third centrifugal force to the third flow rate is between about 100 and about 150.

In some embodiments, the method comprises cryopreserving cells of the output composition to create a cryopreserved composition. In some embodiments, the cryopreserving comprises suspending the cells in a medium comprising a cryoprotectant and freezing the cells. In some embodiments, the freezing is in a controlled rate freezer. In some embodiments, the method involves thawing the cryopreserved cell composition. In some embodiments, the method comprises formulating the thawed cells to make a cell composition for administration as a drug product. In some embodiments, the thawing is done after the cryopreserved cell composition has been frozen for at least 1 day. In some embodiments, the thawing is done after the cryopreserved cell composition has been frozen for at least 2 days. In some embodiments, the thawing is done after the cryopreserved cell composition has been frozen for at least 3 days. In some embodiments, the thawing is done after the cryopreserved cell composition has been frozen for at least 1 week, 10 days, 2 weeks, or 1 month. In some embodiments, the thawing is done after the cryopreserved cell composition has been frozen for up to 10 days, 2 weeks, 1 month, 2 months, 3 months, or 6 months.

### C. Other Processing Steps

In some embodiments, the processing steps for transduction, such as in connection with cell engineering, can additionally include washing, culture, cultivation, stimulation, activation, propagation, and/or formulation of cells. In some embodiments, the genetically engineered cells are subjected to one or more washing steps prior to being collected as an output composition. In some embodiments, the collected output compositions are incubated in the presence of stimulating conditions or a stimulatory agent. Such conditions include those designed to induce proliferation, expansion, activation, and/or survival of cells in the population and/or to mimic antigen exposure. The stimulation can be carried out ex vivo or in vivo after administration to the subject.

i. Washing

In some embodiments, prior to applying the third centrifugal force and the third flow rate to the genetically engineered cells, the method comprises subjecting the genetically engineered cells to one or more washing steps. In some embodiments, the one or more washing steps removes non-viable cells. In some embodiments, the one or more washing steps removes impurities (e.g., proteins, DNA, cell debris, reagents).

In some embodiments, the one or more washing steps comprises applying a centrifugal force of between about 500 G and about 3,000 G. In some embodiments, the centrifugal force is about 500 G. In some embodiments, the centrifugal force is about 1,000 G. In some embodiments, the centrifugal force is about 1,500 G. In some embodiments, the centrifugal force is about 2,000 G. In some embodiments, the centrifugal force is about 2,500 G. In some embodiments, the centrifugal force is about 3,000 G. In some embodiments, the one or more washing steps comprises applying a flow rate of between about 20 mL/min and about 100 mL/min. In some embodiments, the flow rate is about 20 mL/min, about 30 mL/min, about 40 mL/min, about 50 mL/min, about 60 mL/min, about 70 mL/min, about 80 mL/min, about 90 mL/min, or about 100 mL/min. In some embodiments, the flow rate is about 30 mL/min. In some embodiments, the flow rate is about 35 mL/min. In some embodiments, the flow rate is about 40 mL/min. In some embodiments, the flow rate is about 45 mL/min. In some embodiments, the flow rate is about 50 mL/min. In some embodiments, the flow rate is about 55 mL/min. In some embodiments, the flow rate is about 60 mL/min. In some embodiments, the flow rate is about 65 mL/min. In some embodiments, the flow rate is about 70 mL/min. In some embodiments, the flow rate is about 75 mL/min. In some embodiments, the flow rate is about 80 mL/min. In some embodiments, the flow rate is about 85 mL/min. In some embodiments, the flow rate is about 90 mL/min.

In some embodiments, the one or more washing steps comprises applying a centrifugal force (G) and a flow rate (FR) at a ratio of between about 20 G/FR and about 80 G/FR. In some embodiments, the one or more washing steps comprises applying a G/F ratio of about 20, about 25, about 30, about 35, about 40, about 45, about 50, about 55, about 60, about 65, about 70, or about 75. In some embodiments. In some embodiments, the G/FR ratio is about 62.5 G/FR. In some embodiments, the one or more washing steps comprises applying a centrifugal force of about 2,500 G and a flow rate of about 40 mL/min. In some embodiments, the G/FR ratio is about 33.3 G/FR. In some embodiments, the one or more washing steps comprises applying a centrifugal force of about 2,500 G and a flow rate of about 75 mL/min. In some embodiments, the G/FR ratio is about 35 G/FR. In some embodiments, the one or more washing steps comprises applying a centrifugal force of about 1,000 G and a flow rate of about 28.5 mL/min.

In some embodiments, the genetically engineered cells are suspended in media in the centrifuge prior to the one or more washing steps. In some embodiments, the one or more washing steps comprises media exchange. Thus, in some aspects, the media is exchanged during the one or more washing steps for a different solution. In some embodiments, non-viable cells are elutriated during the one or more washing steps. In some embodiments, the non-viable cells are collected as a waste fraction during the one or more washing steps. In some embodiments, the cells of the cell composition are transduced during the one or more washing steps. Thus, in some embodiments, the cells of the cell composition are transduced and washed simultaneously.

ii. Post-Transduction Activation and/or Expansion of Cells

In some embodiments, the cells, e.g., the collected output composition, are further incubated and/or further cultured in connection with genetic engineering. The incubation steps can include culture, cultivation, stimulation, activation, and/or propagation. In some such embodiments, the further incubation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g., stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35 (9): 651-660, Terakura et al. (2012) Blood. 1:72-82, and/or Wang et al. (2012) J Immunother. 35 (9): 689-701.

In some embodiments, the further incubation is carried out in the centrifuge. In some embodiments, the further incubation is carried out without rotation or centrifugation, which generally is carried out subsequent to the collecting of the output composition. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g., automatic transfer, of the cells, e.g., the collected output composition, into a different container or apparatus subsequent to centrifugation.

In some embodiments, the genetically engineered T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of genetically engineered T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing virus-transformed lymphoblastoid cells (LCL) as feeder cells, for example, the virus may be EBV, CMV, or influenza and the transformed LCLs present virally-derived antigen on their surface, optionally in the context of an MHC. In some embodiments, the T cells express a TCR that recognizes a viral antigen. In some embodiments, the viral antigen can be from EBV, CMV, or influenza. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, the further culturing or incubation, e.g., to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days, 14 days, or 15 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g., do not include ex vivo expansion step, or include a substantially shorter ex vivo expansion step.

In some embodiments, the entire process of engineering the cells, e.g., selection and/or enrichment, incubation in connection with transduction and/or further culturing or cultivation is carried out within a time period of more than 9 days, no more than 8 days, no more than 7 days, no more than 6 days, no more than 5 days, no more than 4 days, no more than 3 days, no more than 2 days or no more than 1 days following obtaining a sample from a subject. It is understood that such timing does not include any period of time in which the cells are subjected to cryopreservation.

In some embodiments of the methods provided herein, engineered cells, e.g., output composition or formulated composition, are administered to the subject immediately or shortly after transduction, without significant ex vivo expansion. In some embodiments, the engineered cells can be administered immediately after the transduction step. In some embodiments, the engineered cells can be administered shortly after the transduction step, e.g., with no significant ex vivo expansion or substantially shorter ex vivo expansion than in conventional methods, which can require significant in vitro activation, expansion and/or enrichment. For example, in some embodiments of the methods provided herein, the engineered cells can be administered within three, two or one day of transduction. In some embodiments, the engineered cells can be administered within 48, 36, 24, 20, 16, 12, 8, 4, 2, 1 or fewer hours of the transduction step. In some embodiments, the engineered cells are subject to a substantially shorter in vitro expansion than conventional methods, e.g., for 48, 36, 24, 20, 16, 12, 8, 4, 2, 1 or fewer hours.

In any of such embodiments, expansion and/or activation of cells can occur in vivo after exposure to antigen, e.g., expansion of the engineered cells in the body of the subject after administration of the cells. In some embodiments, the extent, degree or magnitude of in vivo expansion can be further augmented, boosted or enhanced by various methods that are able to modulate, e.g., increase, expansion, proliferation, survival and/or efficacy of the administered cells, e.g., recombinant receptor expressing cells.

In some embodiments, such methods include those involving administration of engineered cells that are further modified with an agent, e.g., nucleic acid, to alter (e.g., increase or decrease) expression or activity of a molecule, in which such altered expression or activity augments, boosts or enhances the expansion, proliferation, survival and/or efficacy of the administered cells. In some embodiments, the expression of the agent, e.g., a nucleic acid, is inducible, repressible, regulatable and/or user controlled, such as by administration of an inducer or other modulating molecule.

In some embodiments, such methods include methods involving the combined administration, e.g., simultaneous or sequential administration, with a drug or agent capable of augmenting, boosting or enhancing the expansion, proliferation, survival and/or efficacy of the administered cells, e.g., recombinant receptor expressing cells.

iii. Formulating

In some embodiments, subsequent to the further incubation, the process for preparing the cells can further include formulating the cells. Thus, among the processing steps may include formulating such compositions.

Also provided are pharmaceutical compositions or formulations for use in such methods, which in some embodiments are formulated in connection with the provided processing methods, such as in the closed system in which other processing steps are carried out, such as in an automated or partially automated fashion.

In some embodiments, the cells and compositions are administered to a subject in the form of a pharmaceutical composition or formulation, such as a composition comprising the cells or cell populations and a pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered.

The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16^{th} edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21^{st} ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### D. Exemplary Features of the Output Composition

In some embodiments, the volume of the output composition (e.g., the collected output composition) comprises between about 1 ml and about 20,000 ml, between about 5 mL and about 2,000 mL, between about 10 mL and about 1,000 mL, between about 15 mL and about 500 mL, or between about 20 mL and about 100 mL. In some embodiments, the volume of the output composition is about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL. In some embodiments, the volume of the output composition is about 1 mL. In some embodiments, the volume of the output composition is about 5 mL. In some embodiments, the volume of the output composition is about 10 mL. In some embodiments, the volume of the output composition is about 15 mL. In some embodiments, the volume of the output composition is about 20 mL. In some embodiments, the volume of the output composition is about 25 mL. In some embodiments, the volume of the output composition is about 30 mL. In some embodiments, the volume of the output composition is about 35 mL. In some embodiments, the volume of the output composition is about 40 mL. In some embodiments, the volume of the output composition is about 45 mL. In some embodiments, the volume of the output composition is about 50 mL.

In some embodiments, the output composition comprises a greater percentage of viable T cells than the cell composition or the input composition. In some embodiments, the output composition comprises a greater percentage of viable T cells than the cell composition. In some embodiments, the percentage of viable T cells in the output composition is at least about 5% greater, at least about 10% greater, at least about 15% greater, at least about 20% greater, or at least about 25% greater than the percentage of viable T cells in the cell composition. In some embodiments, the percentage of viable T cells in the output composition is about 5% greater than the percentage of viable T cells in the cell composition. In some embodiments, the percentage of viable T cells in the output composition is about 10% greater than the percentage of viable T cells in the cell composition. In some embodiments, the percentage of viable T cells in the output composition is about 15% greater than the percentage of viable T cells in the cell composition. In some embodiments, the percentage of viable T cells in the output composition is about 20% greater than the percentage of viable T cells in the cell composition. In some embodiments, the percentage of viable T cells in the output composition is about 25% greater than the percentage of viable T cells in the cell composition. In some embodiments, the percentage of viable T cells in the output composition is about 30% greater than the percentage of viable T cells in the cell composition.

In some embodiments, the output composition comprises a greater percentage of viable T cells than the input composition. In some embodiments, the percentage of viable T cells in the output composition is at least about 5% greater, at least about 10% greater, at least about 15% greater, at least about 20% greater, or at least about 25% greater than the percentage of viable T cells in the input composition. In some embodiments, the percentage of viable T inputs in the output composition is about 5% greater than the percentage of viable T inputs in the input composition. In some embodiments, the percentage of viable T inputs in the output composition is about 10% greater than the percentage of viable T inputs in the input composition. In some embodiments, the percentage of viable T inputs in the output composition is about 15% greater than the percentage of viable T inputs in the input composition. In some embodiments, the percentage of viable T inputs in the output composition is about 20% greater than the percentage of viable T inputs in the input composition. In some embodiments, the percentage of viable T inputs in the output composition is about 25% greater than the percentage of viable T inputs in the input composition. In some embodiments, the percentage of viable T inputs in the output composition is about 30% greater than the percentage of viable T inputs in the input composition.

In particular embodiments, the output composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable cells. In some embodiments, the output composition contains at least at or about 75% viable cells. In certain embodiments, the output composition contains at least at or about 85%, at least at or about 90%, or at least at or about 95% viable cells. In some embodiments, the output composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD3+ T cells. In particular embodiments, the output composition contains at least at or about 75% viable CD3+ T cells. In certain embodiments, the output composition contains at least at or about 85%, at least at or about 90%, or at least at or about 95% viable CD3+ T cells. In some embodiments, the output composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD4+ T cells. In certain embodiments, the output composition contains at least at or about 75% viable CD4+ T cells. In particular embodiments, the output composition contains at least at or about 85%, at least at or about 90%, or at least at or about 95% viable CD4+ T cells. In particular embodiments, the output composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD8+ T cells. In some embodiments, the output composition contains at least at or about 75% viable CD8+ T cells. In certain embodiments, the output composition contains at least at or about 85%, at least at or about 90%, or at least at or about 95% viable CD8+ T cells.

In particular embodiments, the output cells have a low portion and/or frequency of cells that are undergoing and/or are prepared, primed, and/or entering apoptosis. In particular embodiments, the output cells have a low portion and/or frequency of cells that are positive for an apoptotic marker. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the output composition express, contain, and/or are positive for an apoptotic marker. In certain embodiments, less than at or about 25% of the cells of the output composition express, contain, and/or are positive for a marker of apoptosis. In certain embodiments, less than at or about less than at or about 10% cells of the output composition express, contain, and/or are positive for an apoptotic marker. In certain embodiments, less than at or about less than at or about 5% cells of the output composition express, contain, and/or are positive for an apoptotic marker. In certain embodiments, less than at or about less than at or about 1% cells of the output composition express, contain, and/or are positive for an apoptotic marker.

In particular embodiments, the output composition is a composition of cells enriched for CD3+ T cells. In some embodiments, at least or about 60%, at least or about 65%, at least or about 70%, at least or about 75%, at least or about 80%, at least or about 85%, at least or about 90%, at least or about 95%, at least or about 98%, at least or about 98.5%, at least or about 99%, at least or about 99.5%, at least or about 99.9%, 100%, or about 100% of the total cells in the output composition are CD3+ T cells. In some embodiments, at least or about 86%, at least or about 86.5%, at least or about 87%, at least or about 87.5%, at least or about 88%, at least or about 88.5%, at least or about 89%, at least or about 89.5%, at least or about 90%, at least or about 90.5%, at least or about 91%, at least or about 91.5%, at least or about 92%, at least or about 92.5%, at least or about 93%, at least or about 93.5%, at least or about 94%, at least or about 94.5%, at least or about 95%, at least or about 95.5%, at least or about 96%, at least or about 96.5%, at least or about 97%, at least or about 97.5%, at least or about 98%, or at least or about 98.5% of the total cells in the output composition are CD3+ T cells. In some embodiments, between about 80% and about 100%, between about 85% and about 98%, between about 88% and about 96%, or between about 90% and about 94% of the total cells in the output composition are CD3+ T cells. In some embodiments, the output composition consists essentially of CD3+ T cells. In some embodiments, at least or about 90% of the total cells in the output composition are CD3+ T cells and at least or about 40% of the total cells in the output composition express the recombinant receptor (e.g., the CAR).

In certain embodiments, the output composition is a composition of cells enriched for CD4+ T cells and CD8+ T cells. In particular embodiments, CD4+ T cells and CD8+ T cells account for at least or about 60%, at least or about 65%, at least or about 70%, at least or about 75%, at least or about 80%, at least or about 85%, at least or about 90%, at least or about 95%, at least or about 98%, at least or about 98.5%, at least or about 99%, at least or about 99.5%, at least or about 99.9%, 100%, or about 100% of the total cells in the output composition. In some embodiments, CD4+ T cells and CD8+ T cells account for at least or about 86%, at least or about 86.5%, at least or about 87%, at least or about 87.5%, at least or about 88%, at least or about 88.5%, at least or about 89%, at least or about 89.5%, at least or about 90%, at least or about 90.5%, at least or about 91%, at least or about 91.5%, at least or about 92%, at least or about 92.5%, at least or about 93%, at least or about 93.5%, at least or about 94%, at least or about 94.5%, at least or about 95%, at least or about 95.5%, at least or about 96%, at least or about 96.5%, at least or about 97%, at least or about 97.5%, at least or about 98%, or at least or about 98.5% of the total cells in the output composition. In some embodiments, CD4+ T cells and CD8+ T cells account for between about 80% and about 100%, between about 85% and about 98%, between about 88% and about 96%, or between about 90% and about 94% of the total cells in the output composition. In some embodiments, the output composition consists essentially of CD4+ T cells and CD8+ T cells.

In particular embodiments, the output composition contains between at or about 10% and at or about 90%, between at or about 20% and at or about 80%, between at or about 25% and at or about 75%, between at or about 30% and at or about 70%, between at or about 35% and at or about 65%, between at or about 40% and at or about 60%, between at or about 55% and at or about 45%, or about 50% or 50% CD4+ T cells and at or about between at or about 10% and at or about 90%, between at or about 20% and at or about 80%, between at or about 25% and at or about 75%, between at or about 30% and at or about 70%, between at or about 35% and at or about 65%, between at or about 40% and at or about 60%, between at or about 55% and at or about 45%, or about 50% or 50% CD8+ T cells. In certain embodiments, the output composition contains between at or about 35% and at or about 65%, between at or about 40% and at or about 60%, between at or about 55% and at or about 45%, or about 50% or 50% CD4+ T cells and at or about between at or about 35% and at or about 65%, between at or about 40% and at or about 60%, between at or about 55% and at or about 45%, or about 50% or 50% CD8+ T cells. In particular embodiments, the output contains between at or about 35% and at or about 65% CD4+ T cells and at or about between at or about 35% and at or about 65% CD8+ T cells. In particular embodiments, the output composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells to CD8+ T cells. In some embodiments, the composition of cells has a ratio of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3 CD4+ T cells to CD8+ T cells.

In some embodiments, the output composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells that express the recombinant receptor, e.g., the CAR, to CD8+ T cells that express the recombinant receptor, e.g., the CAR. In some embodiments, the ratio of CD4+ T cells that express the recombinant receptor (e.g., the CAR) to CD8+ T cells that express the recombinant receptor (e.g., the CAR) in the output composition is of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3.

In some embodiments, an output composition generated or produced in connection with the provided methods contains cells expressing a recombinant receptor, e.g., a TCR or a CAR. In some embodiments, expressing a recombinant receptor may include, but is not limited to, having one or more recombinant receptor proteins localized at the cell membrane and/or cell surface, having a detectable amount of recombinant receptor protein, having a detectable amount of mRNA encoding the recombinant receptor, having or containing a recombinant polynucleotide that encodes the recombinant receptor, and/or having or containing an mRNA or protein that is a surrogate marker for recombinant receptor expression.

In some embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the output composition express the recombinant receptor. In certain embodiments, at least or about 50% of the cells of the output composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD3+ T cells of the output composition express the recombinant receptor. In some embodiments, at least or about 50% of the CD3+ T cells of the output composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the output composition are CD3+ T cells that express the recombinant receptor. In some embodiments, at least or about 50% of the cells of the output composition are CD3+ T cells that express the recombinant receptor.

In particular embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD4+ T cells of the output composition express the recombinant receptor. In particular embodiments, at least or about 50% of the CD4+ T cells of the output composition express the recombinant receptor. In some embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD8+ T cells of the output composition express the recombinant receptor. In certain embodiments, at least or about 50% of the CD8+ T cells of the output composition express the recombinant receptor.

In particular embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) cells of the output composition are viable cells, e.g., cells negative for an apoptotic marker, such as a caspase (e.g., an activated caspase-3). In certain embodiments, at least at or about 85%, at least at or about 90%, or at least at or about 95% of recombinant receptor-expressing (e.g., CAR+) cells of the output composition are viable cells, e.g., cells negative for an apoptotic marker, such as a caspase (e.g., an activated caspase-3). In some embodiments, at least at or about 90% of recombinant receptor-expressing (e.g., CAR+) cells of the output composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of the output composition are viable cells, e.g., cells negative for an apoptotic marker, such as a caspase (e.g., an activated caspase-3). In certain embodiments, at least at or about 85%, at least at or about 90%, or at least at or about 95% of CD3+ T cells of the output composition are viable cells, e.g., cells negative for an apoptotic marker, such as a caspase (e.g., an activated caspase-3). In particular embodiments, at least at or about 90% of CD3+ T cells of the output composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) CD3+ T cells of the output composition are viable cells, e.g., cells negative for an apoptotic marker, such as a caspase (e.g., an activated caspase-3). In particular embodiments, at least at or about 85%, at least at or about 90%, or at least at or about 95% of recombinant receptor-expressing (e.g., CAR+) CD3+ T cells of the output composition are viable cells, e.g., cells negative for an apoptotic marker, such as a caspase (e.g., an activated caspase-3). In certain embodiments, at least at or about 90% of recombinant receptor-expressing (e.g., CAR+) CD3+ T cells of the output composition are viable cells, e.g., cells negative for an apoptotic marker, such as a caspase (e.g., an activated caspase-3).

In particular embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) cells of a plurality of output compositions produced by the method disclosed herein are viable cells, e.g., cells negative for an apoptotic marker, such as a caspase (e.g., an activated caspase-3). In certain embodiments, on average, at least at or about 85%, at least at or about 90%, or at least at or about 95% of recombinant receptor-expressing (e.g., CAR+) cells of a plurality of output compositions produced by the method disclosed herein are viable cells, e.g., cells negative for an apoptotic marker, such as a caspase (e.g., an activated caspase-3). In some embodiments, on average, at least at or about 90% of recombinant receptor-expressing (e.g., CAR+) cells of a plurality of output compositions produced by the method disclosed herein are viable cells, e.g., cells negative for an apoptotic marker, such as a caspase (e.g., an activated caspase-3). In some embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of a plurality of output compositions produced by the method disclosed herein are viable cells, e.g., cells negative for an apoptotic marker, such as a caspase (e.g., an activated caspase-3). In certain embodiments, on average, at least at or about 85%, at least at or about 90%, or at least at or about 95% of CD3+ T cells of a plurality of output compositions produced by the method disclosed herein are viable cells, e.g., cells negative for an apoptotic marker, such as a caspase (e.g., an activated caspase-3). In particular embodiments, on average, at least at or about 90% of CD3+ T cells of a plurality of output compositions produced by the method disclosed herein are viable cells, e.g., cells negative for an apoptotic marker, such as a caspase (e.g., an activated caspase-3). In some embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) CD3+ T cells of a plurality of output compositions produced by the method disclosed herein are viable cells, e.g., cells negative for an apoptotic marker, such as a caspase (e.g., an activated caspase-3). In particular embodiments, on average, at least 85%, at least 90%, or at least 95% of recombinant receptor-expressing (e.g., CAR+) CD3+ T cells of a plurality of output compositions produced by the method disclosed herein are viable cells, e.g., cells negative for an apoptotic marker, such as a caspase (e.g., an activated caspase-3). In certain embodiments, on average, at least 90% of recombinant receptor-expressing (e.g., CAR+) CD3+ T cells of a plurality of output compositions produced by the method disclosed herein are viable cells, e.g., cells negative for an apoptotic marker, such as a caspase (e.g., an activated caspase-3).

In any of the proceeding embodiments, the plurality of output compositions produced by the method disclosed herein may be originated from the same or different donors. In some aspects, at least two of the plurality of output compositions are originated from different donors. In some aspects, each of the plurality of output compositions is originated from one of a number of different donors, e.g., from about 2, about 5, about 10, about 15, about 20, about 25, about 30, about 35, about 40, about 45, about 50, about 55, about 60, or more than about 60 different donors, e.g., patients in need of a cell therapy such as a CAR-T cell therapy.

In particular embodiments, a majority of the cells of the output composition are naïve or naïve-like cells, central memory cells, and/or effector memory cells. In particular embodiments, a majority of the cells of the output composition are naïve-like or central memory cells. In some embodiments, a majority of the cells of the output composition are central memory cells. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24 (5): 563-571.

In certain embodiments, the cells of the output composition have a high portion and/or frequency of naïve-like T cells or T cells that are surface positive for a marker expressed on naïve-like T cells. In certain embodiments, the cells of the output compositions have a greater portion and/or frequency of naïve-like cells than output compositions generated from alternative processes, such as processes that involve expansion (e.g., processes that include an expansion unit operation and/or include steps intended to cause expansion of cells). In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L-CCR7+.

### II. METHODS OF ENRICHING VIABLE CELLS

Provided are methods for enriching a cell composition (e.g., an engineered cell composition) for viable cells in a centrifuge system. In some embodiments, the centrifuge system is a continuous counterflow elutriation (“CCE”) centrifuge system, also known as a reverse centrifuge system. In some embodiments, the produced cells are for use in cell therapy, such as primary cells prepared for autologous or allogeneic transfer, e.g., in adoptive cell therapy. The methods may include additional cell processing steps, such as cell washing, isolation, separation, collection, formulation, or other steps related to producing a cell composition.

Also provided herein is a method of enriching a cell composition for viable cells comprising (a) applying a first centrifugal force and a first flow rate to a cell composition comprising T cells in a conical fluid enclosure of a centrifuge system to produce a fluidized bed of cells, wherein the cell composition comprises viable and non-viable T cells; and (b) applying a second centrifugal force and a second flow rate to the cell composition, wherein the second centrifugal force and second flow rate recirculate cells of the input composition in a fluid path of the centrifuge system, thereby generating an enriched composition having a higher percentage of viable T cells than the percentage of viable T cells in the cell composition. In some embodiments, the method comprises loading the cell composition (e.g., engineered cell composition) into the centrifuge system, wherein the loading is performed prior to and/or during at least a portion of the applying in (a).

In some embodiments, the centrifuge system includes a cannula within the conical fluid enclosure. In some embodiments, the cannula runs along the length of the conical fluid enclosure. In some embodiments, one end of the cannula is at or near the tip of the conical fluid enclosure. In some embodiments, the other end of the cannula is at or near the wide end of the conical fluid enclosure, such as at or near the center of the wide end.

In some embodiments, the cell composition is loaded into the conical fluid enclosure via the cannula. In some embodiments, the cell composition is loaded into the conical fluid enclosure at or near the tip of the conical fluid enclosure. In some embodiments, the cell composition is loaded into the conical fluid enclosure by entering the end of the cannula at or near the wide end of the conical fluid enclosure and exiting the end of the cannula at or near the tip of the conical fluid enclosure.

In some embodiments, applying the second centrifugal force and second flow rate thereby elutriates out of the conical fluid enclosure a waste fraction of the cell composition. In some embodiments, the elutriated waste fraction has a higher percentage of nonviable T cells than the percentage of nonviable T cells in the cell composition.

In some embodiments, the elutriated cells exit the conical fluid enclosure via an opening at the wide end of the conical fluid enclosure. In some embodiments, the opening at least partially surrounds the end of the cannula at or near the wide end of the conical fluid enclosure. In some embodiments, the opening surrounds the end of the cannula at or near the wide end of the conical fluid enclosure.

In some embodiments, the method involves collecting the elutriated waste fraction. In some embodiments, the elutriated waste fraction is collected in a container. In some embodiments, the container is in fluid communication with the wide end of the conical fluid enclosure.

In some embodiments, applying the second centrifugal force and second flow rate thereby produces within the conical fluid enclosure the enriched composition that has a higher percentage of viable T cells than the percentage of viable T cells in the cell composition.

In some embodiments, the provided methods are used to enrich for viable cells, such as T cells (e.g., engineered T cells). In some embodiments, the cells have been previously introduced with a heterologous polynucleotide encoding an antigen receptor, such as a chimeric antigen receptor (CAR) or a transgenic T cell receptor (TCR). In some embodiments, the cells were previously introduced with a heterologous nucleic acid by a viral vector particle. In some embodiments, the cells have been previously transduced with a viral vector particle containing a heterologous polynucleotide encoding an antigen receptor. In some embodiments, the genetically engineering comprises contacting the T cells of the cell composition with a viral vector particle containing a heterologous polynucleotide encoding the antigen receptor. In some embodiments, the cells express the antigen receptor. In some embodiments, prior to the applying in (a), the method comprises genetically engineering the T cells of the cell composition to express an antigen receptor and/or the T cells of the cell composition have been genetically engineered to express an antigen receptor. Hence, in some embodiments, the provided methods can be used for enrichment of viable T cells that have been previously engineered to express an antigen receptor, such as a transgenic TCR or a CAR.

In some embodiments, the cell composition contains T cells that were cryopreserved and thawed before application of the method In some embodiments, the method involves thawing a cryopreserved cell composition to produce the cell composition comprising T cells.

In some embodiments, the method comprises cryopreserving cells of the enriched composition to create a cryopreserved composition. In some embodiments, the cryopreserving comprises suspending the cells in a medium comprising a cryoprotectant and freezing the cells. In some embodiments, the freezing is in a controlled rate freezer. In some embodiments, the method involves thawing the cryopreserved cell composition. In some embodiments, the method comprises formulating the thawed cells to make a cell composition for administration as a drug product. In some embodiments, the thawing is done after the cryopreserved cell composition has been frozen for at least 1 day. In some embodiments, the thawing is done after the cryopreserved cell composition has been frozen for at least 2 days. In some embodiments, the thawing is done after the cryopreserved cell composition has been frozen for at least 3 days. In some embodiments, the thawing is done after the cryopreserved cell composition has been frozen for at least 1 week, 10 days, 2 weeks, or 1 month. In some embodiments, the thawing is done after the cryopreserved cell composition has been frozen for up to 10 days, 2 weeks, 1 month, 2 months, 3 months, or 6 months.

In some embodiments, the cell composition contains T cells that have not been cryopreserved or thawed before application of the method.

Also provided are populations of cells produced by such methods and methods for using the same.

Thus, in some embodiments, compositions of cells resulting from the provided methods exhibit increased viability following centrifugation, as compared to prior to centrifugation. In some embodiments, the increased viability is observed immediately following centrifugation and/or is maintained for a duration of time following centrifugation (e.g., hours or days).

In some embodiments, the first centrifugal force is between about 500 G and about 5,000 G, between about 500 G and about 4,500 G, between about 500 G and about 4,000 G, between about 500 G and about 3,500 G, between about 500 G and about 3,000 G, between about 500 G and about 2,500 G, between about 500 G and about 2,000 G, between about 500 G and about 1,500 G, between about 500 G and about 1,000 G, between about 1,000 G and about 5,000 G, between about 1,000 G and about 4,500 G, between about 1,000 G and about 4,000 G, between about 1,000 G and about 3,500 G, between about 1,000 G and about 3,000 G, between about 1,000 G and about 2,500 G, between about 1,000 G and about 2,000 G, between about 1,000 G and about 1,500 G, between about 1,500 G and about 5,000 G, between about 1,500 G and about 4,500 G, between about 1,500 G and about 4,000 G, between about 1,500 G and about 3,500 G, between about 1,500 G and about 3,000 G, between about 1,500 G and about 2,500 G, between about 1,500 G and about 2,000 G, between about 2,000 G and about 5,000 G, between about 2,000 G and about 4,500 G, between about 2,000 G and about 4,000 G, between about 2,000 G and about 3,500 G, between about 2,000 G and about 3,000 G, between about 2,000 G and about 2,500 G, between about 2,500 G and about 5,000 G, between about 2,500 G and about 4,500 G, between about 2,500 G and about 4,000 G, between about 2,500 G and about 3,500 G, between about 2,500 G and about 3,000 G, between about 3,000 G and about 5,000 G, between about 3,000 G and about 4,500 G, between about 3,000 G and about 4,000 G, between about 3,000 G and about 3,500 G, between about 3,500 G and about 5,000 G, between about 3,500 G and about 4,500 G, between about 3,500 G and about 4,000 G, between about 4,000 G and about 5,000 G, between about 4,000 G and about 4,500 G, or between or between about 4,500 G and about 5,000 G. In some embodiments, the first centrifugal force is between about 1,000 G and about 4,000 G. In some embodiments, the first centrifugal force is between about 2,000 G and about 4,000 G.

In some embodiments, the first centrifugal force is between about 1,000 G and about 5,000 G, between about 1,500 G and about 4,500 G, between about 2,000 G and about 4,000 G, between about 1,500 G and about 3,500 G, or between about 2,000 G and about 3,000 G. In some embodiments, the first centrifugal force is about 1,000 G. In some embodiments, the first centrifugal force is about 1,500 G. In some embodiments, the first centrifugal force is about 2,000 G. In some embodiments, the first centrifugal force is about 2,500 G. In some embodiments, the first centrifugal force is about 3,000 G. In some embodiments, the first centrifugal force is about 3,500 G. In some embodiments, the first centrifugal force is about 4,000 G. In some embodiments, the first centrifugal force is about 4,500 G. In some embodiments, the first centrifugal force is about 5,000 G.

In some embodiments, the first flow rate is radially inward. In some embodiments, the first flow rate is directed away from the tip of the conical fluid enclosure. In some embodiments, the first centrifugal force is counteracted by the first flow rate. In some embodiments, the first flow rate is a counterflow rate.

In some embodiments, the first flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In some embodiments, the first flow rate is between about 1 mL/min and about 20 mL/min, between about 3 mL/min and about 18 mL/min, between about 5 mL/min and about 15 mL/min, or between about 8 mL/min and about 12 mL/min. In some embodiments, the first flow rate is about 1 mL/min. In some embodiments, the first flow rate is about 3 mL/min. In some embodiments, the first flow rate is about 5 mL/min. In some embodiments, the first flow rate is about 8 mL/min. In some embodiments, the first flow rate is about 9 mL/min. In some embodiments, the first flow rate is about 10 mL/min. In some embodiments, the first flow rate is about 11 mL/min. In some embodiments, the first flow rate is about 12 mL/min. In some embodiments, the first flow rate is about 15 mL/min. In some embodiments, the first flow rate is about 18 mL/min. In some embodiments, the first flow rate is about 20 mL/min.

In some embodiments, the first flow rate is between about 5 mL/min and about 15 mL/min. In some embodiments, (i) the first centrifugal force is between about 1,000 G and about 4,000 G; and (ii) the first flow rate is between about 5 mL/min and about 15 mL/min. In some embodiments, (i) the first centrifugal force is between about 2,000 G and about 4,000 G; and (ii) the first flow rate is between about 5 mL/min and about 15 mL/min.

In some embodiments, the ratio of the first centrifugal force to the first flow rate is between about 100 and about 600, between about 100 and about 500, between about 100 and about 400, between about 100 and about 300, between about 100 and about 200, between about 200 and about 600, between about 200 and about 500, between about 200 and about 400, between about 200 and about 300, between about 300 and about 600, between about 300 and about 500, between about 300 and about 400, between about 400 and about 600, between about 400 and about 500, or between about 500 and about 600. In some embodiments, the ratio of the first centrifugal force to the first flow rate is between about 200 and about 500. In some embodiments, the ratio of the first centrifugal force to the first flow rate is between about 200 and about 400. Ratios of centrifugal force to flow rate herein are ratios of centrifugal force in G to flow rate in mL/min, unless otherwise indicated.

In some embodiments, the ratio of the first centrifugal force to the first flow rate is between about 200 and about 400, between about 225 and about 375, between about 250 and about 350, or between about 275 and about 325. In some embodiments, the ratio of the first centrifugal force to the first flow rate is about 200. In some embodiments, the ratio of the first centrifugal force to the first flow rate is about 200. In some embodiments, the ratio of the first centrifugal force to the first flow rate is about 225. In some embodiments, the ratio of the first centrifugal force to the first flow rate is about 250. In some embodiments, the ratio of the first centrifugal force to the first flow rate is about 275. In some embodiments, the ratio of the first centrifugal force to the first flow rate is about 300. In some embodiments, the ratio of the first centrifugal force to the first flow rate is about 325. In some embodiments, the ratio of the first centrifugal force to the first flow rate is about 350. In some embodiments, the ratio of the first centrifugal force to the first flow rate is about 375. In some embodiments, the ratio of the first centrifugal force to the first flow rate is about 400.

In some embodiments, the first centrifugal force is between about 2,000 G and about 4,000 G and the first flow rate is between about 5 mL/min and about 15 mL/min. In some embodiments, the first centrifugal force is about 3,000 G and the first flow rate is about 10 ml/min.

In some embodiments, the first centrifugal force and the first flow rate are applied to the cell composition (e.g., engineered cell composition) for about 15 seconds, about 30 seconds, about 45 seconds, about 60 seconds, about 2 minutes, about 3 minutes, about 4 minutes, about 5 minutes, about 10 minutes, or about 15 minutes. In some embodiments, the first centrifugal force and the first flow rate are applied to the cell composition for about 15 seconds. In some embodiments, the first centrifugal force and the first flow rate are applied to the cell composition for about 30 seconds. In some embodiments, the first centrifugal force and the first flow rate are applied to the cell composition for about 45 seconds. In some embodiments, the first centrifugal force and the first flow rate are applied to the cell composition for about 60 seconds. In some embodiments, the first centrifugal force and the first flow rate are applied to the cell composition at least until a fluidized bed of cells is established.

In some embodiments, the first centrifugal force and the first flow rate are applied to the cell composition for at least about 15 seconds, at least about 30 seconds, at least about 45 seconds, at least about 60 seconds, at least about 2 minutes, at least about 3 minutes, at least about 4 minutes, at least about 5 minutes, at least about 10 minutes, or at least about 15 minutes. In some embodiments, the first centrifugal force and the first flow rate are applied to the cell composition for at least about 15 seconds. In some embodiments, the first centrifugal force and the first flow rate are applied to the cell composition for at least about 20 seconds. In some embodiments, the first centrifugal force and the first flow rate are applied to the cell composition for at least about 25 seconds. In some embodiments, the first centrifugal force and the first flow rate are applied to the cell composition for at least about 30 seconds. In some embodiments, the first centrifugal force and the first flow rate are applied to the cell composition for at least about 45 seconds. In some embodiments, the first centrifugal force and the first flow rate are applied to the cell composition for at least about 60 seconds. In some embodiments, the first centrifugal force and the first flow rate are applied to the cell composition for at least about 2 minutes. In some embodiments, the first centrifugal force and the first flow rate are applied to the cell composition for at least about 3 minutes. In some embodiments, the first centrifugal force and the first flow rate are applied to the cell composition for at least about 4 minutes. In some embodiments, the first centrifugal force and the first flow rate are applied to the cell composition for at least about 5 minutes. In some embodiments, the first centrifugal force and the first flow rate are applied to the cell composition for at least about 10 minutes. In some embodiments, the first centrifugal force and the first flow rate are applied to the cell composition for at least about 15 minutes.

In some embodiments, the first centrifugal force and the first flow rate are applied to the cell composition for between or between about 15 seconds and 60 seconds. In some embodiments, the first centrifugal force and the first flow rate are applied to the cell composition for between or between about 25 seconds and 60 seconds. In some embodiments, the first centrifugal force and the first flow rate are applied to the cell composition for between or between about 30 seconds and 60 seconds. In some embodiments, the first centrifugal force and the first flow rate are applied to the cell composition for between or between about 15 seconds and 2 minutes. In some embodiments, the first centrifugal force and the first flow rate are applied to the cell composition for between or between about 25 seconds and 2 minutes.

In some embodiments, the first centrifugal force and the first flow rate are applied to the cell composition until a predetermined number of cells are loaded into the conical fluid enclosure. In some embodiments, the first centrifugal force and the first flow rate are applied to the cell composition until a predetermined number of cells are part of the fluidized bed of cells. In some embodiments, the predetermined number of cells is a predetermined number of T cells. In some embodiments, the predetermined number of cells, e.g., T cells, is between about 10 million and 100 million, between about 10 million and 90 million, between about 10 million and 80 million, between about 10 million and 70 million, between about 10 million and 60 million, between about 10 million and 50 million, between about 10 million and 40 million, between about 10 million and 30 million, between about 10 million and 20 million, between about 20 million and 100 million, between about 20 million and 90 million, between about 20 million and 80 million, between about 20 million and 70 million, between about 20 million and 60 million, between about 20 million and 50 million, between about 20 million and 40 million, between about 20 million and 30 million, between about 30 million and 100 million, between about 30 million and 90 million, between about 30 million and 80 million, between about 30 million and 70 million, between about 30 million and 60 million, between about 30 million and 50 million, between about 30 million and 40 million, between about 40 million and 100 million, between about 40 million and 90 million, between about 40 million and 80 million, between about 40 million and 70 million, between about 40 million and 60 million, between about 40 million and 50 million, between about 50 million and 100 million, between about 50 million and 90 million, between about 50 million and 80 million, between about 50 million and 70 million, between about 50 million and 60 million, between about 60 million and 100 million, between about 60 million and 90 million, between about 60 million and 80 million, between about 60 million and 70 million, between about 70 million and 100 million, between about 70 million and 90 million, between about 70 million and 80 million, between about 80 million and 100 million, between about 80 million and 90 million, or between about 90 million and 100 million cells, e.g., T cells. In some embodiments, the predetermined number of cells, e.g., T cells, is about 50 million cells, e.g., T cells.

In some embodiments, the second flow rate is radially inward. In some embodiments, the second flow rate is directed away from the tip of the conical fluid enclosure. In some embodiments, the second centrifugal force is counteracted by the second flow rate. In some embodiments, the second flow rate is a counterflow rate.

In some embodiments, the second flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In other embodiments, the second flow rate is radially outward. In some embodiments, the second flow rate is directed toward the tip of the conical fluid enclosure. In some embodiments, the second centrifugal force and the second flow rate are in the same or substantially the same direction.

In some embodiments, the second flow rate is effected by the flow of media through the conical fluid enclosure. In some embodiments, the flow of media through the conical fluid enclosure is from the wide end to the tip of the conical fluid enclosure. In some embodiments, the media exits the tip of the conical fluid enclosure to enter the cannula.

In some embodiments, the second centrifugal force is between about 100 G and about 4,000 G, between about 100 G and about 3,500 G, between about 100 G and about 3,000 G, between about 100 G and about 2,500 G, between about 100 G and about 2,000 G, between about 100 G and about 1,500 G, between about 100 G and about 1,000 G, between about 100 G and about 500 G, between about 100 G and about 350 G, between about 350 G and about 4,000 G, between about 350 G and about 3,500 G, between about 350 G and about 3,000 G, between about 350 G and about 2,500 G, between about 350 G and about 2,000 G, between about 350 G and about 1,500 G, between about 350 G and about 1,000 G, between about 350 G and about 500 G, between about 500 G and about 4,000 G, between about 500 G and about 3,500 G, between about 500 G and about 3,000 G, between about 500 G and about 2,500 G, between about 500 G and about 2,000 G, between about 500 G and about 1,500 G, between about 500 G and about 1,000 G, between about 1,000 G and about 4,000 G, between about 1,000 G and about 3,500 G, between about 1,000 G and about 3,000 G, between about 1,000 G and about 2,500 G, between about 1,000 G and about 2,000 G, between about 1,000 G and about 1,500 G, between about 1,500 G and about 4,000 G, between about 1,500 G and about 3,500 G, between about 1,500 G and about 3,000 G, between about 1,500 G and about 2,500 G, between about 1,500 G and about 2,000 G, between about 2,000 G and about 4,000 G, between about 2,000 G and about 3,500 G, between about 2,000 G and about 3,000 G, between about 2,000 G and about 2,500 G, between about 2,500 G and about 4,000 G, between about 2,500 G and about 3,500 G, between about 2,500 G and about 3,000 G, between about 3,000 G and about 4,000 G, between about 3,000 G and about 3,500 G, or between about 3,500 G and about 4,000 G.

In some embodiments, the second centrifugal force is between about 350 G and about 4,000 G. In some embodiments, the second centrifugal force is between about 350 G and 3,000 G. In some embodiments, the second centrifugal force is between about 1,500 G and about 3,000 G. In some embodiments, the T cells are activated T cells. In some embodiments, the T cells, e.g., activated T cells, have a mean diameter of about 5 μm to about 25 μm, about 5 μm to about 20 μm, about 5 μm to about 15 μm, about 5 μm to about 10 μm, about 10 μm to about 25 μm, about 10 μm to about 20 μm, about 10 μm to about 15 μm, about 15 μm to about 25 μm, about 15 μm to about 20 μm, or about 20 μm to about 25 μm. In some embodiments, the T cells have a mean diameter of about 9 μm to about 20 μm.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm.

In some embodiments, the second centrifugal force is between about 350 G and about 4,000 G. In some embodiments, the second centrifugal force is between about 350 G and 3,000 G. In some embodiments, the second centrifugal force is between about 500 G and about 1,500 G. In some embodiments, the second centrifugal force is between about 700 G and about 1,300 G. In some embodiments, the second centrifugal force is between about 800 G and about 1,200 G. In some embodiments, the second centrifugal force is between about 900 G and about 1,100 G. In some embodiments, the second centrifugal force is about 1,000 G. In some embodiments, the T cells are non-activated or less activated T cells. In some embodiments, the T cells were cryopreserved and thawed before application of the method. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the second centrifugal force is between about 100 G and about 2,000 G, between about 200 G and about 1800 G, between about 500 G and about 1,500 G, or between about 750 G and about 1,250 G. In some embodiments, the second centrifugal force is about 250 G. In some embodiments, the second centrifugal force is about 500 G. In some embodiments, the second centrifugal force is about 600 G. In some embodiments, the second centrifugal force is about 700 G. In some embodiments, the second centrifugal force is about 800 G. In some embodiments, the second centrifugal force is about 900 G. In some embodiments, the second centrifugal force is about 1,000 G. In some embodiments, the second centrifugal force is about 1,100 G. In some embodiments, the second centrifugal force is about 1,200 G. In some embodiments, the second centrifugal force is about 1,300 G. In some embodiments, the second centrifugal force is about 1,400 G. In some embodiments, the second centrifugal force is about 1,500 G. In some embodiments, the second centrifugal force is about 1,300 G. In some embodiments, the second centrifugal force is about 1,750 G. In some embodiments, the second centrifugal force is about 2,000 G.

In some embodiments, the second flow rate is between about 5 mL/min and about 100 mL/min, between about 5 mL/min and about 90 mL/min, between about 5 mL/min and about 80 mL/min, between about 5 mL/min and about 70 mL/min, between about 5 mL/min and about 60 mL/min, between about 5 mL/min and about 50 mL/min, between about 5 mL/min and about 40 mL/min, between about 5 mL/min and about 30 mL/min, between about 5 mL/min and about 20 mL/min, between about 5 mL/min and about 10 mL/min, between about 10 mL/min and about 100 mL/min, between about 10 mL/min and about 90 mL/min, between about 10 mL/min and about 80 mL/min, between about 10 mL/min and about 70 mL/min, between about 10 mL/min and about 60 mL/min, between about 10 mL/min and about 50 mL/min, between about 10 mL/min and about 40 mL/min, between about 10 mL/min and about 30 mL/min, between about 10 mL/min and about 20 mL/min, between about 20 mL/min and about 100 mL/min, between about 20 mL/min and about 90 mL/min, between about 20 mL/min and about 80 mL/min, between about 20 mL/min and about 70 mL/min, between about 20 mL/min and about 60 mL/min, between about 20 mL/min and about 50 mL/min, between about 20 mL/min and about 40 mL/min, between about 20 mL/min and about 30 mL/min, between about 30 mL/min and about 100 mL/min, between about 30 mL/min and about 90 mL/min, between about 30 mL/min and about 80 mL/min, between about 30 mL/min and about 70 mL/min, between about 30 mL/min and about 60 mL/min, between about 30 mL/min and about 50 mL/min, between about 30 mL/min and about 40 mL/min, between about 40 mL/min and about 100 mL/min, between about 40 mL/min and about 90 mL/min, between about 40 mL/min and about 80 mL/min, between about 40 mL/min and about 70 mL/min, between about 40 mL/min and about 60 mL/min, between about 40 mL/min and about 50 mL/min, between about 50 mL/min and about 100 mL/min, between about 50 mL/min and about 90 mL/min, between about 50 mL/min and about 80 mL/min, between about 50 mL/min and about 70 mL/min, between about 50 mL/min and about 60 mL/min, between about 60 mL/min and about 100 mL/min, between about 60 mL/min and about 90 mL/min, between about 60 mL/min and about 80 mL/min, between about 60 mL/min and about 70 mL/min, between about 70 mL/min and about 100 mL/min, between about 70 mL/min and about 90 mL/min, between about 70 mL/min and about 80 mL/min, between about 80 mL/min and about 100 mL/min, between about 80 mL/min and about 90 mL/min, or between about 90 mL/min and about 100 mL/min.

In some embodiments, the second flow rate is between about 5 mL/min and about 100 mL/min. In some embodiments, (i) the second centrifugal force is between about 350 G and about 4,000 G; and (ii) the second flow rate is between about 5 mL/min and about 100 mL/min. In some embodiments, the second flow rate is between about 10 mL/min and about 65 mL/min. In some embodiments, the second flow rate is between about 10 mL/min and about 35 mL/min. In some embodiments, the T cells are activated T cells. In some embodiments, the T cells, e.g., activated T cells, have a mean diameter of about 5 μm to about 25 μm, about 5 μm to about 20 μm, about 5 μm to about 15 μm, about 5 μm to about 10 μm, about 10 μm to about 25 μm, about 10 μm to about 20 μm, about 10 μm to about 15 μm, about 15 μm to about 25 μm, about 15 μm to about 20 μm, or about 20 μm to about 25 μm. In some embodiments, the T cells have a mean diameter of about 9 μm to about 20 μm.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm.

In some embodiments, the second flow rate is 30 mL/min or less. In some embodiments, the second flow rate is between about 25 mL/min and about 30 mL/min. In some embodiments, (i) the second centrifugal force is between about 500 G and about 1,500 G; and (ii) the second flow rate is between about 25 mL/min and about 30 mL/min. In some embodiments, the T cells are non-activated or less activated T cells. In some embodiments, the T cells were cryopreserved and thawed before application of the method. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the second flow rate is 30 mL/min or less. In some embodiments, the second flow rate is between about 25 mL/min and about 30 mL/min. In some embodiments, (i) the second centrifugal force is between about 500 G and about 1,500 G; and (ii) the second flow rate is between about 25 mL/min and about 30 mL/min. In some embodiments, the T cells are non-activated or less activated T cells. In some embodiments, the method comprises activating and culturing the cells of the cell composition (e.g., culturing the cells for at least 24, 48, 72, or 96 hours) before applying the first centrifugal force and the first flow rate to the cell composition. In some embodiments, the method comprises cryopreserving and optionally thawing T cells of the enriched composition. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the second flow rate is between about 65 mL/min and about 100 mL/min. In some embodiments, (i) the second centrifugal force is between about 1,500 G and about 3,000 G; and (ii) the second flow rate is between about 65 mL/min and about 100 mL/min. In some embodiments, the second flow rate is between about 65 mL/min and about 90 mL/min. In some embodiments, the second flow rate is between about 65 mL/min and about 80 mL/min. In some embodiments, the T cells are non-activated or less activated T cells. In some embodiments, the T cells were cryopreserved and thawed before application of the method. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the second flow rate is between about 65 mL/min and about 100 mL/min. In some embodiments, (i) the second centrifugal force is between about 1,500 G and about 3,000 G; and (ii) the second flow rate is between about 65 mL/min and about 100 mL/min. In some embodiments, the second flow rate is between about 65 mL/min and about 90 mL/min. In some embodiments, the second flow rate is between about 65 mL/min and about 80 mL/min. In some embodiments, the T cells are non-activated or less activated T cells. In some embodiments, the method comprises cryopreserving and optionally thawing cells of the enriched composition. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the second flow rate is between about 10 and about 100 mL/min, between about 15 and about 90 mL/min, between about 20 and about 80 mL/min, between about 25 and about 70 mL/min, between about 30 and about 60 mL/min, or between about 35 and about 50 mL/min. In some embodiments, the second flow rate is about 20 mL/min, about 21 mL/min, about 22 mL/min, about 23 mL/min, about 24 mL/min, about 25 mL/min, about 25.5 mL/min, about 26 mL/min, about 26.5 mL/min, about 27 mL/min, about 27.5 mL/min, about 28 mL/min, about 28.5 mL/min, about 29 mL/min, about 29.5 mL/min, about 30 mL/min, about 31 mL/min, about 32 mL/min, about 33 mL/min, about 34 mL/min, or about 35 mL/min. In some embodiments, the second flow rate is about 25 mL/min. In some embodiments, the second flow rate is about 25.5 mL/min. In some embodiments, the second flow rate is about 26 mL/min. In some embodiments, the second flow rate is about 26.5 mL/min. In some embodiments, the second flow rate is about 27 mL/min. In some embodiments, the second flow rate is about 27.5 mL/min. In some embodiments, the second flow rate is about 28 mL/min. In some embodiments, the second flow rate is about 28.5 mL/min. In some embodiments, the second flow rate is about 29 mL/min. In some embodiments, the second flow rate is about 29.5 mL/min. In some embodiments, the second flow rate is about 30 mL/min.

In some embodiments, the ratio of the second centrifugal force to the second flow rate is between about 20 and about 100, between about 20 and about 90, between about 20 and about 80, between about 20 and about 70, between about 20 and about 60, between about 20 and about 50, between about 20 and about 40, between about 20 and about 30, between about 30 and about 100, between about 30 and about 90, between about 30 and about 80, between about 30 and about 70, between about 30 and about 60, between about 30 and about 50, between about 30 and about 40, between about 40 and about 100, between about 40 and about 90, between about 40 and about 80, between about 40 and about 70, between about 40 and about 60, between about 40 and about 50, between about 50 and about 100, between about 50 and about 90, between about 50 and about 80, between about 50 and about 70, between about 50 and about 60, between about 60 and about 100, between about 60 and about 90, between about 60 and about 80, between about 60 and about 70, between about 70 and about 100, between about 70 and about 90, between about 70 and about 80, between about 80 and about 100, between about 80 and about 90, or between about 90 and about 100.

In some embodiments, the ratio of the second centrifugal force to the second flow rate is between about 30 and about 70. In some embodiments, the ratio of the second centrifugal force to the second flow rate is between about 30 and about 40. In some embodiments, the T cells are activated T cells. In some embodiments, the T cells, e.g., activated T cells, have a mean diameter of about 5 μm to about 25 μm, about 5 μm to about 20 μm, about 5 μm to about 15 μm, about 5 μm to about 10 μm, about 10 μm to about 25 μm, about 10 μm to about 20 μm, about 10 μm to about 15 μm, about 15 μm to about 25 μm, about 15 μm to about 20 μm, or about 20 μm to about 25 μm. In some embodiments, the T cells have a mean diameter of about 9 μm to about 20 μm.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm.

In some embodiments, the T cells have a mean diameter of about 9 μm to about 20 μm, and the ratio of the second centrifugal force to the second flow rate is between about 30 and about 70. In some embodiments, the T cells have a mean diameter of 10 μm to 20 μm (e.g., 12 μm to 20 μm or 14 μm to 20 μm), and the ratio of the second centrifugal force to the second flow rate is between about 30 and about 70. In some embodiments, the T cells have a mean diameter of 12 μm to 20 μm, and the ratio of the second centrifugal force to the second flow rate is between about 30 and about 70. In some embodiments, the T cells have a mean diameter of 14 μm to 20 μm, and the ratio of the second centrifugal force to the second flow rate is between about 30 and about 70.

In some embodiments, the T cells have a mean diameter of less than 9 μm, and the ratio of the second centrifugal force to the second flow rate is between about 30 and about 40.

In some embodiments, the ratio of the second centrifugal force to the second flow rate is between about 30 and about 40. In some embodiments, the T cells are non-activated or less activated T cells. In some embodiments, the T cells were cryopreserved and thawed before application of the method. In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the ratio of the second centrifugal force to the second flow rate is between about 20 and about 100, between about 25 and about 80, or between about 30 and about 60. In some embodiments, the ratio of the second centrifugal force to the second flow rate is about 20. In some embodiments, the ratio of the second centrifugal force to the second flow rate is about 25. In some embodiments, the ratio of the second centrifugal force to the second flow rate is about 30. In some embodiments, the ratio of the second centrifugal force to the second flow rate is about 35. In some embodiments, the ratio of the second centrifugal force to the second flow rate is about 40. In some embodiments, the ratio of the second centrifugal force to the second flow rate is about 45. In some embodiments, the ratio of the second centrifugal force to the second flow rate is about 50. In some embodiments, the ratio of the second centrifugal force to the second flow rate is about 55. In some embodiments, the ratio of the second centrifugal force to the second flow rate is about 60. In some embodiments, the ratio of the second centrifugal force to the second flow rate is about 65. In some embodiments, the ratio of the second centrifugal force to the second flow rate is about 70. In some embodiments, the ratio of the second centrifugal force to the second flow rate is about 75.

In some embodiments, the second centrifugal force is between about 500 G and about 1,500 G and the second flow rate is between about 25 mL/min and about 30 mL/min. In some embodiments, the second centrifugal force is about 1,000 G and the flow rate is about 28.5 mL/min.

In some embodiments, the second centrifugal force and the second flow rate are applied to the cell composition for between about 5 minutes and about 100 minutes, between about 10 minutes and about 90 minutes, between about 15 minutes and about 80 minutes, between about 20 minutes and about 70 minutes, between about 25 minutes and about 60 minutes, between about 30 minutes and about 50 minutes, or between about 35 minutes and about 40 minutes. In some embodiments, the second centrifugal force and the second flow rate are applied to the cell composition for about 5 minutes. In some embodiments, the second centrifugal force and the second flow rate are applied to the cell composition for about 10 minutes. In some embodiments, the second centrifugal force and the second flow rate are applied to the cell composition for about 15 minutes. In some embodiments, the second centrifugal force and the second flow rate are applied to the cell composition for about 20 minutes. In some embodiments, the second centrifugal force and the second flow rate are applied to the cell composition for about 25 minutes. In some embodiments, the second centrifugal force and the second flow rate are applied to the cell composition for about 30 minutes. In some embodiments, the second centrifugal force and the second flow rate are applied to the cell composition for about 45 minutes. In some embodiments, the second centrifugal force and the second flow rate are applied to the cell composition for about 60 minutes. In some embodiments, the second centrifugal force and the second flow rate are applied to the cell composition for about 75 minutes. In some embodiments, the second centrifugal force and the second flow rate are applied to the cell composition for about 90 minutes.

In some embodiments, the second centrifugal force and the second flow rate are applied to the cell composition for at least about 15 minutes. In some embodiments, the second centrifugal force and the second flow rate are applied to the cell composition for at least about 30 minutes. In some embodiments, the second centrifugal force and the second flow rate are applied to the cell composition for at least about 45 minutes. In some embodiments, the second centrifugal force and the second flow rate are applied to the cell composition for at least about 60 minutes. In some embodiments, the second centrifugal force and the second flow rate are applied to the cell composition for at least about 75 minutes. In some embodiments, the second centrifugal force and the second flow rate are applied to the cell composition for at least about 90 minutes.

### A. Cell Composition (e.g., Engineered Cell Composition)

In some embodiments, a cell composition comprising T cells (e.g., T cells previously engineered to express an antigen receptor) is enriched for viable cells in a continuous counterflow centrifuge system. In some embodiments, the concentration of cells of the engineered cell composition is from or from about 1.0×10⁵ cells/mL to 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the engineered cell composition comprises about 1×10⁶ cells/mL. In some embodiments, the engineered cell composition comprises about 1.25×10⁶ cells/mL. In some embodiments, the cell composition comprises about 1.5×10⁶ cells/mL. In some embodiments, the engineered cell composition comprises about 1.75×10⁶ cells/mL. In some embodiments, the engineered cell composition comprises about 2×10⁶ cells/mL. In some embodiments, the engineered cell composition comprises about 2.25×10⁶ cells/mL. In some embodiments, the engineered cell composition comprises about 2.5×10⁶ cells/mL. In some embodiments, the engineered cell composition comprises about 2.75×10⁶ cells/mL. In some embodiments, the engineered cell composition comprises about 3×10⁶ cells/mL.

In some embodiments, the volume of the engineered cell composition comprises between about 20 mL and about 300 mL, between about 25 mL and about 250 mL, between about 30 mL and about 200 mL, between about 35 mL and between about 150 mL, or between about 40 mL and about 100 mL. In some embodiments, the volume of the engineered cell composition is about 20 mL. In some embodiments, the volume of the engineered cell composition is about 25 mL. In some embodiments, the volume of the engineered cell composition is about 30 mL. In some embodiments, the volume of the engineered cell composition is about 35 mL. In some embodiments, the volume of the engineered cell composition is about 40 mL. In some embodiments, the volume of the engineered cell composition is about 45 mL. In some embodiments, the volume of the engineered cell composition is about 50 mL. In some embodiments, the volume of the engineered cell composition is about 55 mL. In some embodiments, the volume of the engineered cell composition is about 60 mL. In some embodiments, the volume of the engineered cell composition is about 70 mL. In some embodiments, the volume of the engineered cell composition is about 80 mL. In some embodiments, the volume of the engineered cell composition is about 100 mL. In some embodiments, the volume of the engineered cell composition is about 125 mL. In some embodiments, the volume of the engineered cell composition is about 150 mL. In some embodiments, the volume of the engineered cell composition is about 175 mL. In some embodiments, the volume of the engineered cell composition is about 200 mL.

In some embodiments, the engineered cell composition comprises about 1×10⁸ total cells, about 2×10⁸ total cells, about 3×10⁸ total cells, about 4×10⁸ total cells, about 5×10⁸ total cells, about 6×10⁸ total cells, about 7×10⁸ total cells, about 8×10⁸ total cells, about 9×10⁸ total cells, or about 1×10⁹ total cells.

In some embodiments, the T cells have a mean diameter of about 5 μm to about 25 μm, about 5 μm to about 20 μm, about 5 μm to about 15 μm, about 5 μm to about 10 μm, about 10 μm to about 25 μm, about 10 μm to about 20 μm, about 10 μm to about 15 μm, about 15 μm to about 25 μm, about 15 μm to about 20 μm, or about 20 μm to about 25 μm. In some embodiments, the T cells have a mean diameter of about 9 μm to about 20 μm. In some embodiments, the T cells are activated T cells.

In some embodiments, the T cells have a mean diameter of about 10 μm to about 20 μm. In some embodiments, the T cells have a mean diameter of about 12 μm to about 20 μm. In some embodiments, the T cells have a mean diameter of about 14 μm to about 20 μm. In some embodiments, the T cells are activated T cells.

In some embodiments, the T cells have a mean diameter of less than 9 μm. In some embodiments, the T cells have a mean diameter of about 3 μm to about 9 μm, about 4 μm to about 9 μm, about 5 μm to about 9 μm, about 6 μm to about 9 μm, about 7 μm to about 9 μm, or about 8 μm to about 9 μm. In some embodiments, the T cells have a mean diameter of about 6 μm to about 9 μm.

In some embodiments, the cells are incubated and/or cultured prior to genetic engineering in accord with the provided methods. The incubation steps can include culture, cultivation, stimulation, activation, and/or propagation. In some embodiments, the method comprises incubating the T cells of a cell composition under stimulating conditions prior to genetic engineering. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the T cells of the cell composition are incubated under stimulating conditions prior to the applying the first centrifugal force and the first flow rate. In some embodiments, the method comprises incubating the T cells of the cell composition under stimulating conditions prior to the loading the cell composition into the centrifuge system. In some embodiments, the T cells of the cell composition are incubated under stimulating conditions prior to the loading the cell composition into the centrifuge system. In some embodiments, the cell composition comprises activated T cells. In some embodiments, the cell composition comprises T cells expressing HLA-DR, CD25, CD69, CD71, CD40L, 4-1BB, or a combination thereof.

In some embodiments, the stimulating conditions comprise the presence of a stimulatory reagent. In some embodiments, the stimulatory reagent is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. In some embodiments, the stimulatory reagent is capable of activating one or more intracellular signaling domains of one or more components of a TCR complex and one or more intracellular signaling domains of one or more costimulatory molecules. In some embodiments, the stimulatory reagent comprises (i) a primary agent that specifically binds to a member of a TCR complex; and (ii) a secondary agent that specifically binds to a T cell costimulatory molecule. In some embodiments, the primary agent specifically binds to CD3. In some embodiments, the costimulatory the costimulatory molecule is selected from CD28, CD137 (4-1-BB), OX40 or ICOS. In some embodiments, at least one of the primary and secondary agents comprises an antibody or an antigen-binding fragment thereof. In some embodiments, the primary agent is or comprises an anti-CD3 antibody or antigen-binding fragment thereof. In some embodiments, the secondary agent is or comprises an anti-CD28 antibody or antigen-binding fragment thereof. Optionally, the expansion method may further comprise the step of adding anti-CD3 and/or anti CD28 antibody to the culture medium (e.g., at a concentration of at least about 0.5 ng/ml).

In some embodiments, the stimulating agents include IL-2 and/or IL-15, for example, an IL-2 concentration of at least about 10 units/mL. In some aspects, incubation is carried out in accordance with techniques such as those described in U.S. Pat. No. 6,040,177 to Riddell et al., Klebanoff et al. (2012) J Immunother. 35 (9): 651-660, Terakura et al. (2012) Blood. 1:72-82, and/or Wang et al. (2012) J Immunother. 35 (9): 689-701.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some cases, a viral vector particle may be used that does not require that the cells, e.g., T cells, are activated. In some such instances, the cells may be selected and/or transduced prior to activation and or in the absence of activation.

In some embodiments, at least 40%, 50%, 60%, 70%, 80%, 90% or more of the cells, e.g., T cells, in the cell composition are activated, such as, in some cases, are surface positive for one or more of HLA-DR, CD25, CD69, CD71, CD40L and/or 4-1BB. In some embodiments, cells are activated with an activating agent, such as in the presence of anti-CD3/anti-CD28, prior to initiation of the applying the first centrifugal force and the first flow rate, e.g., prior to establishment of the fluidized bed and/or prior to initiation of transduction. Methods of expanding T cell populations in vitro in the absence of exogenous growth factors or low amounts of exogenous growth factors are known in the art (see, e.g., U.S. Pat. No. 6,352,694 B1 and European Patent EP 0 700 430 B1). In general, such methods employ a solid phase surfaces of greater than 1 μM to which various bind agents (e.g., anti-CD3 antibody and/or anti-CD28 antibody) are immobilized. For example, Dynabeads® CD3/CD28 (Invitrogen) are commercially available reagents for T cell expansion, which are uniform, 4.5 μm superparamagnetic, sterile, non-pyrogenic polystyrene beads coated with a mixture of affinity purified monoclonal antibodies against the CD3 and CD28 cell surface molecules on human T cells In some embodiments, the activating agent, e.g., anti-CD3 and/or anti-CD28, can be immobilized on beads, such as magnetic beads.

In some embodiments, the cell activation is also performed in the presence IL-2 (e.g., from or from about 50 IU/mL to 200 IU/mL, such as or about 100 IU/mL). In some embodiments, the activation is carried out between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the activation is carried out at a temperature greater than or greater than about 25° C., such as generally greater than or greater than about 32° C., 35° C. or 37° C., for example at or about 37° C.±2° C., such as at a temperature of at or about 37° C.

In some embodiments, cells are not activated with an activating agent, such as in the presence of anti-CD3/anti-CD28, prior to initiation of the contacting, e.g., prior to initiation of transduction. In some embodiments, the cell composition comprises a plurality of resting cells. In some embodiments, at least 40%, 50%, 60%, 70%, 80%, 90% or more of the T cells in the population are resting T cells, such as T cells that lack a T cell activation marker, such as a surface marker or intracellular cytokine or other marker, and/or T cells that are in the G0 or G0G1a stage of the cell cycle.

In particular aspects, the provided methods allow transduction to happen in T cells without the need for activation prior to the contacting and/or incubation with the oligomeric protein reagent, such as multimerization reagent. In some embodiments, the methods include transducing a population of T cells that contain resting or naïve T cells with a viral vector without first, e.g., prior to the transduction, activating and/or stimulating the T cells. In some such embodiments, the provided methods can be used to prepare cells, such as T cells, for adoptive therapy, that do not include a step of activating and/or stimulating T cells.

In some embodiments, the cells generally are eukaryotic cells, such as mammalian cells, and typically are human cells. In some embodiments, the cells are derived from the blood, bone marrow, lymph, or lymphoid organs, or are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells are primary T cells. In some embodiments, the cells are primary T cells from a human subject. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells comprise or are enriched for CD3+ T cells. In some embodiments, the cells comprise or are enriched for CD4+ T cells. In some embodiments, the cells comprise or are enriched for CD8+ T cells. In some embodiments the cells comprise CD4+ and CD8+ T cells. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, preparation of the cells includes one or more culture and/or preparation steps. The cells may be isolated from a sample, such as a biological sample, e.g., one obtained from or derived from a subject. In some embodiments, the subject from which the cells are isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In some embodiments, the cells are PBMCs. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, it is not necessary to enrich or select cells prior to performing the provided method.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. Separation methods may include any of those disclosed herein, including methods using reversible reagent systems, e.g., agents (such as receptor binding agents or selection agents) and reagents as described herein.

In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some embodiments, the cell composition is enriched for CD3+ T cells. In some embodiments, at least 70%, at least 75%, at least 80%, at least 80%, at least 85%, at least 90%, at least 95%, or at least 99% of the cells in the cell composition are CD3+ T cells. In some embodiments, the cell composition is enriched for CD4+ T cells. In some embodiments, at least 70%, at least 75%, at least 80%, at least 80%, at least 85%, at least 90%, at least 95%, or at least 99% of the cells in the cell composition are CD4+ T cells. In some embodiments, the cell composition is enriched for CD8+ T cells. In some embodiments, at least 70%, at least 75%, at least 80%, at least 80%, at least 85%, at least 90%, at least 95%, or at least 99% of the cells in the cell composition are CD8+ T cells. In some embodiments, the cell composition is enriched for CD4+ and CD8+ T cells. In some embodiments, at least 70%, at least 75%, at least 80%, at least 80%, at least 85%, at least 90%, at least 95%, or at least 99% of the cells in the cell composition are CD4+ or CD8+ T cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakuraet al. (2012) Blood. 1:72-82; Wang et al. (2012) J Immunother. 35 (9): 689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead contains a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5): 355-376. In both cases, cells can be labeled with multiple markers, allowing for the isolation of well-defined T cell subsets at high purity.

In some embodiments, the preparation methods include steps for freezing, e.g., cryopreserving, the cells, either before or after isolation, incubation, and/or genetic engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

### B. Enriched Composition

In some embodiments, the methods comprise applying a third centrifugal force and a third flow rate to the enriched composition to produce an enriched composition comprising the viability enriched cells (e.g., comprising engineered cells). In some embodiments, the enriched composition is collected or harvested, such as for downstream use in a cell therapy. In some embodiments, the third centrifugal force and the third flow rate and applied to the cells of the enriched composition in the conical fluid enclosure of the centrifuge system. In some embodiments, the application of the third centrifugal force and the third flow rate to the enriched cells allows for the collection or harvest of the enriched composition.

In some embodiments, the enriched composition is collected via the cannula. In some embodiments, the enriched composition enters the end of the cannula at or near the tip of the conical fluid enclosure and exits the end of the cannula at or near the wide end of the conical fluid enclosure.

In some embodiments, the method involves cryopreserving the enriched composition. In some embodiments, the level of viable cells in the enriched composition is maintained in the cryopreserved enriched composition. In some embodiments, the method involves thawing the cryopreserved enriched composition. In some embodiments, the level of viable cells in the enriched composition is maintained in the thawed enriched composition.

In some embodiments, the third centrifugal force is between about 2,000 G and about 3,000 G, between about 2,200 G and about 2,800 G, or between about 2,400 G and about 2,600 G. In some embodiments, the third centrifugal force is about 2,000 G. In some embodiments, the third centrifugal force is about 2,100 G. In some embodiments, the third centrifugal force is about 2,200 G. In some embodiments, the third centrifugal force is about 2,300 G. In some embodiments, the third centrifugal force is about 2,400 G. In some embodiments, the third centrifugal force is about 2,500 G. In some embodiments, the third centrifugal force is about 2,600 G. In some embodiments, the third centrifugal force is about 2,700 G. In some embodiments, the third centrifugal force is about 2,800 G. In some embodiments, the third centrifugal force is about 2,900 G. In some embodiments, the third centrifugal force is about 3,000 G.

In some embodiments, the third flow rate is radially outward. In some embodiments, the third flow rate is directed toward the tip of the conical fluid enclosure. In some embodiments, the third centrifugal force and the third flow rate are in the same or substantially the same direction.

In some embodiments, the third flow rate is effected by the flow of media through the conical fluid enclosure. In some embodiments, the flow of media through the conical fluid enclosure is from the wide end to the tip of the conical fluid enclosure. In some embodiments, the media exits the tip of the conical fluid enclosure to enter the cannula.

In some embodiments, the third flow rate is between about 10 mL/min and about 30 mL/min, between about 12 mL/min and about 28 mL/min, between about 15 mL/min, and about 25 mL/min, or between about 18 mL/min and about 22 mL/min. In some embodiments, the third flow rate is about 15 mL/min. In some embodiments, the third flow rate is about 16 mL/min. In some embodiments, the third flow rate is about 17 mL/min. In some embodiments, the third flow rate is about 18 mL/min. In some embodiments, the third flow rate is about 19 mL/min. In some embodiments, the third flow rate is about 20 mL/min. In some embodiments, the third flow rate is about 21 mL/min. In some embodiments, the third flow rate is about 22 mL/min. In some embodiments, the third flow rate is about 23 mL/min. In some embodiments, the third flow rate is about 24 mL/min. In some embodiments, the third flow rate is about 15 mL/min. In some embodiments, the third flow rate is about 25 mL/min.

In some embodiments, the ratio of the third centrifugal force to the third flow rate is between about 100 and about 150, between about 110 and 140, or between about 120 and 130. In some embodiments, the ratio of the third centrifugal force to the third flow rate is about 100. In some embodiments, the ratio of the third centrifugal force to the third flow rate is about 105. In some embodiments, the ratio of the third centrifugal force to the third flow rate is about 110. In some embodiments, the ratio of the third centrifugal force to the third flow rate is about 115. In some embodiments, the ratio of the third centrifugal force to the third flow rate is about 120. In some embodiments, the ratio of the third centrifugal force to the third flow rate is about 125. In some embodiments, the ratio of the third centrifugal force to the third flow rate is about 130. In some embodiments, the ratio of the third centrifugal force to the third flow rate is about 135. In some embodiments, the ratio of the third centrifugal force to the third flow rate is about 140. In some embodiments, the ratio of the third centrifugal force to the third flow rate is about 145. In some embodiments, the ratio of the third centrifugal force to the third flow rate is about 150.

In some embodiments, the third centrifugal force is between about 2,000 G and about 3,000 G; and the third flow rate is between about 15 mL/min and about 25 mL/min. In some embodiments, the third centrifugal force is about 2,500 G and the third flow rate is about 20 mL/min.

In some embodiments, the processing steps for can additionally include washing, culture, cultivation, stimulation, activation, propagation, and/or formulation of cells. In some embodiments, the enriched cells are subjected to one or more washing steps prior to being collected as an output composition. In some embodiments, the collected enriched compositions cells are incubated in the presence of stimulating conditions or a stimulatory agent. Such conditions include those designed to induce proliferation, expansion, activation, and/or survival of cells in the population and/or to mimic antigen exposure. The stimulation can be carried out ex vivo or in vivo after administration to the subject. In some embodiments, the enriched composition is collected and subjected to further processing steps, including any of those described in Section I.C.

### C. Exemplary Features of the Enriched Composition

In some embodiments, the volume of the enriched composition (e.g., the collected enriched composition) comprises between about 1 ml and about 20,000 ml, between about 5 mL and about 2,000 mL, between about 10 mL and about 1,000 mL, between about 15 mL and about 500 mL, or between about 20 mL and about 100 mL. In some embodiments, the volume of the enriched composition is about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL. In some embodiments, the volume of the enriched composition is about 1 mL. In some embodiments, the volume of the enriched composition is about 5 mL. In some embodiments, the volume of the enriched composition is about 10 mL. In some embodiments, the volume of the enriched composition is about 15 mL. In some embodiments, the volume of the enriched composition is about 20 mL. In some embodiments, the volume of the enriched composition is about 25 mL. In some embodiments, the volume of the enriched composition is about 30 mL. In some embodiments, the volume of the enriched composition is about 35 mL. In some embodiments, the volume of the enriched composition is about 40 mL. In some embodiments, the volume of the enriched composition is about 45 mL. In some embodiments, the volume of the enriched composition is about 50 mL.

In some embodiments, the enriched composition comprises a greater percentage of viable T cells than the cell composition or the cell composition. In some embodiments, the enriched composition comprises a greater percentage of viable T cells than the cell composition. In some embodiments, the percentage of viable T cells in the enriched composition is at least about 5% greater, at least about 10% greater, at least about 15% greater, at least about 20% greater, or at least about 25% greater than the percentage of viable T cells in the cell composition. In some embodiments, the percentage of viable T cells in the enriched composition is about 5% greater than the percentage of viable T cells in the cell composition. In some embodiments, the percentage of viable T cells in the enriched composition is about 10% greater than the percentage of viable T cells in the cell composition. In some embodiments, the percentage of viable T cells in the enriched composition is about 15% greater than the percentage of viable T cells in the cell composition. In some embodiments, the percentage of viable T cells in the enriched composition is about 20% greater than the percentage of viable T cells in the cell composition. In some embodiments, the percentage of viable T cells in the enriched composition is about 25% greater than the percentage of viable T cells in the cell composition. In some embodiments, the percentage of viable T cells in the enriched composition is about 30% greater than the percentage of viable T cells in the cell composition.

In some embodiments, the enriched composition comprises a greater percentage of viable T cells than the cell composition (e.g., the engineered cell composition). In some embodiments, the percentage of viable T cells in the enriched composition is at least about 5% greater, at least about 10% greater, at least about 15% greater, at least about 20% greater, or at least about 25% greater than the percentage of viable T cells in the cell composition. In some embodiments, the percentage of viable T inputs in the enriched composition is about 5% greater than the percentage of viable T inputs in the cell composition. In some embodiments, the percentage of viable T inputs in the enriched composition is about 10% greater than the percentage of viable T inputs in the cell composition. In some embodiments, the percentage of viable T inputs in the enriched composition is about 15% greater than the percentage of viable T inputs in the cell composition. In some embodiments, the percentage of viable T inputs in the enriched composition is about 20% greater than the percentage of viable T inputs in the cell composition. In some embodiments, the percentage of viable T inputs in the enriched composition is about 25% greater than the percentage of viable T inputs in the cell composition. In some embodiments, the percentage of viable T inputs in the enriched composition is about 30% greater than the percentage of viable T inputs in the cell composition.

In particular embodiments, the enriched composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable cells. In some embodiments, the enriched composition contains at least at or about 75% viable cells. In certain embodiments, the enriched composition contains at least at or about 85%, at least at or about 90%, or at least at or about 95% viable cells. In some embodiments, the enriched composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD3+ T cells. In particular embodiments, the enriched composition contains at least at or about 75% viable CD3+ T cells. In certain embodiments, the enriched composition contains at least at or about 85%, at least at or about 90%, or at least at or about 95% viable CD3+ T cells. In some embodiments, the enriched composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD4+ T cells. In certain embodiments, the enriched composition contains at least at or about 75% viable CD4+ T cells. In particular embodiments, the enriched composition contains at least at or about 85%, at least at or about 90%, or at least at or about 95% viable CD4+ T cells. In particular embodiments, the enriched composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD8+ T cells. In some embodiments, the enriched composition contains at least at or about 75% viable CD8+ T cells. In certain embodiments, the enriched composition contains at least at or about 85%, at least at or about 90%, or at least at or about 95% viable CD8+ T cells.

In particular embodiments, the output cells have a low portion and/or frequency of cells that are undergoing and/or are prepared, primed, and/or entering apoptosis. In particular embodiments, the output cells have a low portion and/or frequency of cells that are positive for an apoptotic marker. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the enriched composition express, contain, and/or are positive for an apoptotic marker. In certain embodiments, less than at or about 25% of the cells of the enriched composition express, contain, and/or are positive for a marker of apoptosis. In certain embodiments, less than at or about less than at or about 10% cells of the enriched composition express, contain, and/or are positive for an apoptotic marker. In certain embodiments, less than at or about less than at or about 5% cells of the enriched composition express, contain, and/or are positive for an apoptotic marker. In certain embodiments, less than at or about less than at or about 1% cells of the enriched composition express, contain, and/or are positive for an apoptotic marker.

In particular embodiments, the enriched composition is a composition of cells enriched for CD3+ T cells. In some embodiments, at least or about 60%, at least or about 65%, at least or about 70%, at least or about 75%, at least or about 80%, at least or about 85%, at least or about 90%, at least or about 95%, at least or about 98%, at least or about 98.5%, at least or about 99%, at least or about 99.5%, at least or about 99.9%, 100%, or about 100% of the total cells in the enriched composition are CD3+ T cells. In some embodiments, at least or about 86%, at least or about 86.5%, at least or about 87%, at least or about 87.5%, at least or about 88%, at least or about 88.5%, at least or about 89%, at least or about 89.5%, at least or about 90%, at least or about 90.5%, at least or about 91%, at least or about 91.5%, at least or about 92%, at least or about 92.5%, at least or about 93%, at least or about 93.5%, at least or about 94%, at least or about 94.5%, at least or about 95%, at least or about 95.5%, at least or about 96%, at least or about 96.5%, at least or about 97%, at least or about 97.5%, at least or about 98%, or at least or about 98.5% of the total cells in the enriched composition are CD3+ T cells. In some embodiments, between about 80% and about 100%, between about 85% and about 98%, between about 88% and about 96%, or between about 90% and about 94% of the total cells in the enriched composition are CD3+ T cells. In some embodiments, the enriched composition consists essentially of CD3+ T cells. In some embodiments, at least or about 90% of the total cells in the enriched composition are CD3+ T cells and at least or about 40% of the total cells in the enriched composition express the recombinant receptor (e.g., the CAR).

In certain embodiments, the enriched composition is a composition of cells enriched for CD4+ T cells and CD8+ T cells. In particular embodiments, CD4+ T cells and CD8+ T cells account for at least or about 60%, at least or about 65%, at least or about 70%, at least or about 75%, at least or about 80%, at least or about 85%, at least or about 90%, at least or about 95%, at least or about 98%, at least or about 98.5%, at least or about 99%, at least or about 99.5%, at least or about 99.9%, 100%, or about 100% of the total cells in the enriched composition. In some embodiments, CD4+ T cells and CD8+ T cells account for at least or about 86%, at least or about 86.5%, at least or about 87%, at least or about 87.5%, at least or about 88%, at least or about 88.5%, at least or about 89%, at least or about 89.5%, at least or about 90%, at least or about 90.5%, at least or about 91%, at least or about 91.5%, at least or about 92%, at least or about 92.5%, at least or about 93%, at least or about 93.5%, at least or about 94%, at least or about 94.5%, at least or about 95%, at least or about 95.5%, at least or about 96%, at least or about 96.5%, at least or about 97%, at least or about 97.5%, at least or about 98%, or at least or about 98.5% of the total cells in the enriched composition. In some embodiments, CD4+ T cells and CD8+ T cells account for between about 80% and about 100%, between about 85% and about 98%, between about 88% and about 96%, or between about 90% and about 94% of the total cells in the enriched composition. In some embodiments, the enriched composition consists essentially of CD4+ T cells and CD8+ T cells.

In particular embodiments, the enriched composition contains between at or about 10% and at or about 90%, between at or about 20% and at or about 80%, between at or about 25% and at or about 75%, between at or about 30% and at or about 70%, between at or about 35% and at or about 65%, between at or about 40% and at or about 60%, between at or about 55% and at or about 45%, or about 50% or 50% CD4+ T cells and at or about between at or about 10% and at or about 90%, between at or about 20% and at or about 80%, between at or about 25% and at or about 75%, between at or about 30% and at or about 70%, between at or about 35% and at or about 65%, between at or about 40% and at or about 60%, between at or about 55% and at or about 45%, or about 50% or 50% CD8+ T cells. In certain embodiments, the enriched composition contains between at or about 35% and at or about 65%, between at or about 40% and at or about 60%, between at or about 55% and at or about 45%, or about 50% or 50% CD4+ T cells and at or about between at or about 35% and at or about 65%, between at or about 40% and at or about 60%, between at or about 55% and at or about 45%, or about 50% or 50% CD8+ T cells. In particular embodiments, the output contains between at or about 35% and at or about 65% CD4+ T cells and at or about between at or about 35% and at or about 65% CD8+ T cells. In particular embodiments, the enriched composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells to CD8+ T cells. In some embodiments, the composition of cells has a ratio of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3 CD4+ T cells to CD8+ T cells.

In some embodiments, the enriched composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells that express the recombinant receptor, e.g., the CAR, to CD8+ T cells that express the recombinant receptor, e.g., the CAR. In some embodiments, the ratio of CD4+ T cells that express the recombinant receptor (e.g., the CAR) to CD8+ T cells that express the recombinant receptor (e.g., the CAR) in the enriched composition is of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3.

In some embodiments, an enriched composition generated or produced in connection with the provided methods contains cells expressing a recombinant receptor, e.g., a TCR or a CAR. In some embodiments, expressing a recombinant receptor may include, but is not limited to, having one or more recombinant receptor proteins localized at the cell membrane and/or cell surface, having a detectable amount of recombinant receptor protein, having a detectable amount of mRNA encoding the recombinant receptor, having or containing a recombinant polynucleotide that encodes the recombinant receptor, and/or having or containing an mRNA or protein that is a surrogate marker for recombinant receptor expression.

In some embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the enriched composition express the recombinant receptor. In certain embodiments, at least or about 50% of the cells of the enriched composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD3+ T cells of the enriched composition express the recombinant receptor. In some embodiments, at least or about 50% of the CD3+ T cells of the enriched composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the enriched composition are CD3+ T cells that express the recombinant receptor. In some embodiments, at least or about 50% of the cells of the enriched composition are CD3+ T cells that express the recombinant receptor.

In particular embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD4+ T cells of the enriched composition express the recombinant receptor. In particular embodiments, at least or about 50% of the CD4+ T cells of the enriched composition express the recombinant receptor. In some embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD8+ T cells of the enriched composition express the recombinant receptor. In certain embodiments, at least or about 50% of the CD8+ T cells of the enriched composition express the recombinant receptor.

In particular embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) cells of the enriched composition are viable cells, e.g., cells negative for an apoptotic marker, such as a caspase (e.g., an activated caspase-3). In certain embodiments, at least at or about 85%, at least at or about 90%, or at least at or about 95% of recombinant receptor-expressing (e.g., CAR+) cells of the enriched composition are viable cells, e.g., cells negative for an apoptotic marker, such as a caspase (e.g., an activated caspase-3). In some embodiments, at least at or about 90% of recombinant receptor-expressing (e.g., CAR+) cells of the enriched composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of the enriched composition are viable cells, e.g., cells negative for an apoptotic marker, such as a caspase (e.g., an activated caspase-3). In certain embodiments, at least at or about 85%, at least at or about 90%, or at least at or about 95% of CD3+ T cells of the enriched composition are viable cells, e.g., cells negative for an apoptotic marker, such as a caspase (e.g., an activated caspase-3). In particular embodiments, at least at or about 90% of CD3+ T cells of the enriched composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) CD3+ T cells of the enriched composition are viable cells, e.g., cells negative for an apoptotic marker, such as a caspase (e.g., an activated caspase-3). In particular embodiments, at least at or about 85%, at least at or about 90%, or at least at or about 95% of recombinant receptor-expressing (e.g., CAR+) CD3+ T cells of the enriched composition are viable cells, e.g., cells negative for an apoptotic marker, such as a caspase (e.g., an activated caspase-3).

In certain embodiments, at least at or about 90% of recombinant receptor-expressing (e.g., CAR+) CD3+ T cells of the enriched composition are viable cells, e.g., cells negative for an apoptotic marker, such as a caspase (e.g., an activated caspase-3).

In particular embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) cells of a plurality of enriched compositions produced by the method disclosed herein are viable cells, e.g., cells negative for an apoptotic marker, such as a caspase (e.g., an activated caspase-3). In certain embodiments, on average, at least at or about 85%, at least at or about 90%, or at least at or about 95% of recombinant receptor-expressing (e.g., CAR+) cells of a plurality of enriched compositions produced by the method disclosed herein are viable cells, e.g., cells negative for an apoptotic marker, such as a caspase (e.g., an activated caspase-3). In some embodiments, on average, at least at or about 90% of recombinant receptor-expressing (e.g., CAR+) cells of a plurality of enriched compositions produced by the method disclosed herein are viable cells, e.g., cells negative for an apoptotic marker, such as a caspase (e.g., an activated caspase-3). In some embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of a plurality of enriched compositions produced by the method disclosed herein are viable cells, e.g., cells negative for an apoptotic marker, such as a caspase (e.g., an activated caspase-3). In certain embodiments, on average, at least at or about 85%, at least at or about 90%, or at least at or about 95% of CD3+ T cells of a plurality of enriched compositions produced by the method disclosed herein are viable cells, e.g., cells negative for an apoptotic marker, such as a caspase (e.g., an activated caspase-3). In particular embodiments, on average, at least at or about 90% of CD3+ T cells of a plurality of enriched compositions produced by the method disclosed herein are viable cells, e.g., cells negative for an apoptotic marker, such as a caspase (e.g., an activated caspase-3). In some embodiments, on average, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) CD3+ T cells of a plurality of enriched compositions produced by the method disclosed herein are viable cells, e.g., cells negative for an apoptotic marker, such as a caspase (e.g., an activated caspase-3). In particular embodiments, on average, at least 85%, at least 90%, or at least 95% of recombinant receptor-expressing (e.g., CAR+) CD3+ T cells of a plurality of enriched compositions produced by the method disclosed herein are viable cells, e.g., cells negative for an apoptotic marker, such as a caspase (e.g., an activated caspase-3). In certain embodiments, on average, at least 90% of recombinant receptor-expressing (e.g., CAR+) CD3+ T cells of a plurality of enriched compositions produced by the method disclosed herein are viable cells, e.g., cells negative for an apoptotic marker, such as a caspase (e.g., an activated caspase-3).

In any of the proceeding embodiments, the plurality of enriched compositions produced by the method disclosed herein may be originated from the same or different donors. In some aspects, at least two of the plurality of enriched compositions are originated from different donors. In some aspects, each of the plurality of enriched compositions is originated from one of a number of different donors, e.g., from about 2, about 5, about 10, about 15, about 20, about 25, about 30, about 35, about 40, about 45, about 50, about 55, about 60, or more than about 60 different donors, e.g., patients in need of a cell therapy such as a CAR-T cell therapy.

In particular embodiments, a majority of the cells of the enriched composition are naïve or naïve-like cells, central memory cells, and/or effector memory cells. In particular embodiments, a majority of the cells of the enriched composition are naïve-like or central memory cells. In some embodiments, a majority of the cells of the enriched composition are central memory cells. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24 (5): 563-571.

In certain embodiments, the cells of the enriched composition have a high portion and/or frequency of naïve-like T cells or T cells that are surface positive for a marker expressed on naïve-like T cells. In certain embodiments, the cells of the enriched compositions have a greater portion and/or frequency of naïve-like cells than enriched compositions generated from alternative processes, such as processes that involve expansion (e.g., processes that include an expansion unit operation and/or include steps intended to cause expansion of cells). In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L-CCR7+.

### III. METHODS OF DEBEADING CELLULAR COMPOSITIONS

Provided herein are methods of removing beads or “debeading” a cell composition using a centrifuge system (e.g., a “reverse” or continuous counterflow centrifuge system). In some embodiments, the cell composition comprises any of those as described in Section I.A.i, such as cell compositions that have been previously incubated with beads (e.g., paramagnetic beads). Thus, in some embodiments, the cell composition comprises cells (e.g., T cells) and beads (e.g., paramagnetic beads).

In some embodiments, the beads are paramagnetic beads, such as paramagnetic polystyrene beads. In some embodiments, the cells were previously subjected to incubation with the beads, e.g., to activate or stimulate the cells. In some embodiments, the beads are coated with a stimulatory reagent. In some embodiments, the stimulatory reagent is capable of activating one or more intracellular signaling domains of one or more components of a TCR complex and one or more intracellular signaling domains of one or more costimulatory molecules. In some embodiments, the stimulatory reagent comprises (i) a primary agent that specifically binds to a member of a TCR complex; and (ii) a secondary agent that specifically binds to a T cell costimulatory molecule. In some embodiments, the primary agent specifically binds to CD3. In some embodiments, the costimulatory the costimulatory molecule is selected from CD28, CD137 (4-1-BB), OX40 or ICOS. In some embodiments, at least one of the primary and secondary agents comprises an antibody or an antigen-binding fragment thereof. In some embodiments, the primary agent is or comprises an anti-CD3 antibody or antigen-binding fragment thereof. In some embodiments, the secondary agent is or comprises an anti-CD28 antibody or antigen-binding fragment thereof. In some embodiments, the beads comprise beads coated with anti-CD3 antibodies. In some embodiments, the beads comprise beads coated with anti-CD28 antibodies. In some embodiments, the beads comprise beads coated with anti-CD3 antibodies and beads coated with anti-CD28 antibodies.

In some embodiments, the cell composition comprises at least about 10×10⁶ beads, at least about 25×10⁶ beads, at least about 50×10⁶ beads, at least about 75×10⁶ beads, at least about 100×10⁶ beads, at least about 125×10⁶ beads, at least about 150×10⁶ beads, at least about 175×10⁶ beads, at least about 200×10⁶ beads, at least about 225×10⁶ beads, at least about 250×10⁶ beads, at least about 275×10⁶ beads, or at least about 300×10⁶ beads. In some embodiments, the cell composition comprises about 100×10⁶ beads. In some embodiments, the cell composition comprises about 150×10⁶ beads. In some embodiments, the cell composition comprises about 200×10⁶ beads. In some embodiments, the cell composition comprises about 250×10⁶ beads. In some embodiments, the cell composition comprises about 300×10⁶ beads.

In some embodiments, the method comprises (i) applying a first centrifugal force and a first flow rate to establish a fluidized bed of cells; and (ii) applying a second centrifugal force and a second flow rate to remove beads from the cell composition, thereby generating a debeaded composition.

In some embodiments, the method includes loading the cell composition into the conical fluid enclosure prior to applying the first centrifugal force and the first flow rate.

In some embodiments, the centrifuge system includes a cannula within the conical fluid enclosure. In some embodiments, the cannula runs along the length of the conical fluid enclosure. In some embodiments, one end of the cannula is at or near the tip of the conical fluid enclosure. In some embodiments, the other end of the cannula is at or near the wide end of the conical fluid enclosure, such as at or near the center of the wide end.

In some embodiments, the cell composition is loaded into the conical fluid enclosure via the cannula. In some embodiments, the cell composition is loaded into the conical fluid enclosure at or near the tip of the conical fluid enclosure. In some embodiments, the cell composition is loaded into the conical fluid enclosure by entering the end of the cannula at or near the wide end of the conical fluid enclosure and exiting the end of the cannula at or near the tip of the conical fluid enclosure.

In some embodiments, the beads are removed from the cell composition by elutriating cells from the conical fluid enclosure. In some embodiments, the elutriated cells exit the conical fluid enclosure via an opening at the wide end of the conical fluid enclosure. In some embodiments, the opening at least partially surrounds the end of the cannula at or near the wide end of the conical fluid enclosure. In some embodiments, the opening surrounds the end of the cannula at or near the wide end of the conical fluid enclosure.

In some embodiments, the beads remain in the conical fluid enclosure. In some embodiments, the beads are removed from the conical fluid enclosure following the elutriation of the cells. In some embodiments, the beads are removed via the cannula. In some embodiments, the beads enter the end of the cannula at or near the tip of the conical fluid enclosure and exit the end of the cannula at or near the wide end of the conical fluid enclosure.

In some embodiments, the first centrifugal force is between about 500 G and about 2,000 G, or between about 750 G and about 1,500 G or between about 500 G and about 1,000 G. In some embodiments, the first centrifugal force is about 500 G. In some embodiments, the first centrifugal force is about 1,000 G. In some embodiments, the first centrifugal force is about 1,500 G. In some embodiments, the first centrifugal force is about 2,000 G.

In some embodiments, the first flow rate is radially inward. In some embodiments, the first flow rate is directed away from the tip of the conical fluid enclosure. In some embodiments, the first centrifugal force is counteracted by the first flow rate. In some embodiments, the first flow rate is a counterflow rate.

In some embodiments, the first flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In some embodiments, the first flow rate is between about 10 mL/min and about 50 mL/min, between about 15 mL/min and about 45 mL/min, between about 20 mL/min and about 40 mL/min, or between about 25 mL/min and about 35 mL/min. In some embodiments, the first flow rate is about 20 mL/min. In some embodiments, the first flow rate is about 25 mL/min. In some embodiments, the first flow rate is about 30 mL/min. In some embodiments, the first flow rate is about 35 mL/min. In some embodiments, the first flow rate is about 40 mL/min. In some embodiments, the first flow rate is about 45 mL/min. In some embodiments, the first flow rate is about 50 mL/min.

In some embodiments, the first centrifugal force is about 1,000 G and the first flow rate is about 30 mL/min.

In some embodiments, the second centrifugal force is between about 100 G and about 1,000 G, between about 200 G and about 800 G, or between about 400 G and about 600 G. In some embodiments, the second centrifugal force is about 100 G. In some embodiments, the second centrifugal force is about 200 G. In some embodiments, the second centrifugal force is about 300 G. In some embodiments, the second centrifugal force is about 400 G. In some embodiments, the second centrifugal force is about 500 G. In some embodiments, the second centrifugal force is about 600 G. In some embodiments, the second centrifugal force is about 700 G. In some embodiments, the second centrifugal force is about 800 G.

In some embodiments, the second flow rate is radially inward. In some embodiments, the second flow rate is directed away from the tip of the conical fluid enclosure. In some embodiments, the second centrifugal force is counteracted by the second flow rate. In some embodiments, the second flow rate is a counterflow rate.

In some embodiments, the second flow rate is effected by the flow of media through the cannula. In some embodiments, the flow of media through the cannula is from the wide end to the tip of the conical fluid enclosure. In some embodiments, the media exits the cannula to enter the conical fluid enclosure at its tip.

In some embodiments, the second flow rate is between about 10 mL/min and about 100 mL/min, between about 20 mL/min and about 80 mL/min, or between about 30 mL/min and about 60 mL/min. In some embodiments, the second flow rate is about 10 mL/min. In some embodiments, the second flow rate is about 20 mL/min. In some embodiments, the second flow rate is about 30 mL/min. In some embodiments, the second flow rate is about 40 mL/min. In some embodiments, the second flow rate is about 50 mL/min. In some embodiments, the second flow rate is about 60 mL/min. In some embodiments, the second flow rate is about 70 mL/min. In some embodiments, the second flow rate is about 80 mL/min. In some embodiments, the second flow rate is about 90 mL/min. In some embodiments, the second flow rate is about 100 mL/min.

In some embodiments, the second centrifugal force is about 600 G and the second flow rate is about 50 mL/min.

In some embodiments, the application of the second centrifugal force and the second flow rate removes at least about 50%, at least about 60%, at least about 70%, at least about 80%, at least about 80%, at least about 90%, at least about 95%, or at least about 99% of beads from the cell composition.

In some embodiments, the debeaded composition contains less than about 10%, less than about 5%, less than about 2%, less than about 1%, less than about 0.5%, less than about 0.2%, less than about 0.1%, less than about 0.05%, less than about 0.02%, or less than about 0.01% of the beads contained in the cell composition. In some embodiments, the debeaded composition contains about 10% of the beads contained in the cell composition. In some embodiments, the debeaded composition contains about 5% of the beads contained in the cell composition. In some embodiments, the debeaded composition contains about 1% of the beads contained in the cell composition. In some embodiments, the debeaded composition contains about 1% of the beads contained in the cell composition. In some embodiments, the debeaded composition contains about 0.5% of the beads contained in the cell composition. In some embodiments, the debeaded composition contains about 0.1% of the beads contained in the cell composition. In some embodiments, the debeaded composition contains about 0.05% of the beads contained in the cell composition. In some embodiments, the debeaded composition contains about 0.01% of the beads contained in the cell composition. In some embodiments, the debeaded composition is collected as a debeaded output composition. In some embodiments, the methods include subject the debeaded output composition to one or more additional processing steps, such as transduction, washing, culture, cultivation, stimulation, propagation, and/or formulation of cells. In some embodiments, the debeaded output composition is subsequently subjected to transduction, such as for use in cell therapy.

### IV. VIRAL VECTOR PARTICLES

In some embodiments, the viral vector particle is a retroviral vector particle, such as a lentiviral particle. In some embodiments, the viral vector particle contains a nucleic acid encoding a recombinant and/or heterologous molecule, e.g., recombinant or heterologous protein, such as a recombinant and/or heterologous receptor, such as chimeric antigen receptor (CAR) or other antigen receptor, in a genome of the viral vector. In some embodiments, the recombinant molecule is a chemokine, a chemokine receptor, a cytokine, a cytokine receptor, an antigen receptor (e.g., a CAR or a TCR), or a combination thereof. In some embodiments, the recombinant molecule is a chemokine. In some embodiments, the recombinant molecule is a chemokine receptor. In some embodiments, the recombinant molecule is a cytokine. In some embodiments, the recombinant molecule is a cytokine receptor. In some embodiments, the recombinant molecule is an antigen receptor (e.g., a CAR or a TCR). In some embodiments, the antigen receptor is a chimeric antigen receptor (CAR). In some embodiments, the antigen receptor is a T cell receptor (TCR). The genome of the viral vector particle typically includes sequences in addition to the nucleic acid encoding the recombinant molecule. Such sequences may include sequences that allow the genome to be packaged into the virus particle and/or sequences that promote expression of a nucleic acid encoding a recombinant receptor, such as a CAR.

### A. Viral Vector

In some embodiments, the viral vector particle contains a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72:9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1 and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

In some embodiments, the viral vector contains a nucleic acid that encodes a heterologous recombinant protein. In some embodiments, the heterologous recombinant molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

In some embodiments, the viral vector contains a nucleic acid that encodes a recombinant receptor and/or chimeric receptor, such as a heterologous receptor protein. The recombinant receptor, such as heterologous receptor, may include antigen receptors, such as functional non-TCR antigen receptors, including chimeric antigen receptors (CARs), and other antigen-binding receptors such as transgenic T cell receptors (TCRs). The receptors may also include other receptors, such as other chimeric receptors, such as receptors that bind to particular ligands and having transmembrane and/or intracellular signaling domains similar to those present in a CAR.

In any of such examples, the nucleic acid is inserted or located in a region of the viral vector, such as generally in a non-essential region of the viral genome. In some embodiments, the nucleic acid is inserted into the viral genome in the place of certain viral sequences to produce a virus that is replication defective.

In some embodiments, the encoded recombinant antigen receptor, e.g., CAR, is one that is capable of specifically binding to one or more ligand on a cell or disease to be targeted, such as a cancer, infectious disease, inflammatory or autoimmune disease, or other disease or condition, including those described herein for targeting with the provided methods and compositions.

In certain embodiments, an exemplary antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the exemplary antigens are orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by and/or characteristic of or specific for HIV, HCV, HBV, HPV, and/or other pathogens and/or oncogenic versions thereof.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigen receptors, including CARs and recombinant TCRs, and production and introduction thereof, in some embodiments include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PloS ONE 8 (4): e61338; Turtle et al., Curr. Opin. Immunol., 2012 October; 24 (5): 633-39; Wu et al., Cancer, 2012 Mar. 18 (2): 160-75.

i. Chimeric Antigen Receptors (CARs)

In some embodiments, the nucleic acid contained in a genome of the viral vector encodes a chimeric antigen receptor (CAR). The CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In some embodiments, CARs are constructed with a specificity for a particular marker, such as a marker expressed in a particular cell type to be targeted by adoptive therapy, e.g., a cancer marker and/or any of the antigens described. Thus, the CAR typically includes one or more antigen-binding fragment, domain, or portion of an antibody, or one or more antibody variable domains, and/or antibody molecules. In some embodiments, the CAR includes an antigen-binding portion or portions of an antibody molecule, such as a variable heavy chain (VH) or antigen-binding portion thereof, or a single-chain antibody fragment (scFv) derived from the variable heavy (VH) and variable light (VL) chains of a monoclonal antibody (mAb).

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g., a cytoplasmic signaling domain or region of a zeta chain of a CD3-zeta (CD35) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g., antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PloS ONE 8 (4): e61338; Turtle et al., Curr. Opin. Immunol., 2012 October; 24 (5): 633-39; Wu et al., Cancer, 2012 Mar. 18 (2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35 (9): 689-701; and Brentjens et al., Sci Transl Med. 2013 5 (177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

Exemplary antigen receptors, including CARs, and methods for engineering and introducing such receptors into cells, include those described, for example, in international patent application publication numbers WO2000/14257, WO2013/126726, WO2012/129514, WO2014/031687, WO2013/166321, WO2013/071154, WO2013/123061, WO2016/0046724, WO2016/014789, WO2016/090320, WO2016/094304, WO2017/025038, and WO2017/173256, U.S. patent application publication numbers US2002131960, US2013287748, and US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov. 2013 April; 3 (4): 388-398; Davila et al. (2013) PLoS ONE 8 (4): e61338; Turtle et al., Curr. Opin. Immunol., 2012 October; 24 (5): 633-39; and Wu et al., Cancer, 2012 Mar. 18 (2): 160-75. In some aspects, the antigen receptors include a CAR as described in U.S. Pat. No. 7,446,190, and those described in International Patent Application Publication No. WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35 (9): 689-701; and Brentjens et al., Sci Transl Med. 2013 5 (177). See also WO2014031687, U.S. Pat. Nos. 8,339,645, 7,446,179, US 2013/0149337, U.S. Pat. Nos. 7,446,190, and 8,389,282.

Exemplary antigen receptors, e.g., CARs, also include any described in Marofi et al., Stem Cell Res Ther 12:81 (2021); Townsend et al., J Exp Clin Cancer Res 37:163 (2018); Ma et al., Int J Biol Sci 15 (12): 2548-2560 (2019); Zhao and Cao, Front Immunol 10:2250 (2019); Han et al., J Cancer 12 (2): 326-334 (2021); Specht et al., Cancer Res 79:4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement_1): 1932 (2019); or can contain any of the antibodies or antigen-binding fragments described in U.S. Pat. Nos. 8,153,765; 8,603,477, and 8,008,450; U.S. Pub. No. US20120189622 and US20100260748; and International PCT Publication Nos. WO2006099875, WO2009080829, WO2012092612, and WO2014210064.

Further exemplary antigen receptors, e.g., CARs, such as anti-BCMA CARs, include the CARs of idecabtagene vicleucel, ABECMAC, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allo1 (Poseida), Allo-715 (Pfizer/Allogene), CT053 (Carsgen), Descartes-08 (Cartesian), PHE885 (Novartis), ARI-002 (Hospital Clinic Barcelona, IDIBAPS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary antigen receptors, e.g., CARs, also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134 (Supplement_1): 593; and Abramson et al., 2020, The Lancet 396 (10254): 839-852). In some of any of the provided embodiments, the CAR is the CAR of TECARTUS™ (brexucabtagene autoleucel, see Mian and Hill, 2021, Expert Opin Biol Ther; 21 (4): 435-441; and Wang et al., 2021, Blood 138 (Supplement 1): 744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55 (4): 466-479; Mueller et al., 2021, Blood Adv. 5 (23): 4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377 (26): 2531-2544; Jacobson et al., 2021, The Lancet 23 (1): P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398 (10297): 314-324; and Martin, Abstract #549 [Oral], presented at 2021 American Society of Hematology (ASH) Annual Meeting & Exposition)).

In some embodiments, the antigen is BCMA. In some embodiments, the CAR includes a BCMA-binding portion or portions of the antibody molecule, such as a heavy chain variable (VH) region and/or light chain variable (VL) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin. Cancer Res., 2013, 19 (8): 2048-2060; Feng et al., Scand. J. Immunol. (2020) 92: e12910; U.S. Pat. Nos. 9,034,324 9,765,342; U.S. Patent Publication Nos. US2016/0046724, US20170183418; and International PCT Application Nos. WO 2016090320, WO2016090327, WO2016094304, WO2016014565, WO2016014789, WO2010104949, WO2017025038, WO2017173256, WO2018085690, or WO2021091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO2017025038 or WO2018028647. In some embodiments, the anti-BCMA CAR contains two single-domain anti-BCMA antibodies. In some embodiments, the two single-domain anti-BCMA antibodies are derived from one or more antibodies described in WO2017025038 or WO2018028647. In some embodiments, the BCMA binding domain comprises or consists of A37353-G4S-A37917 (G4S being a linker between the two binding domains), described in WO2017025038 or WO2018028647, and provided, e.g., in SEQ ID NOs: 300, 301 and 302 of WO2017025038 or WO2018028647 (with or without signal peptide). In some embodiments, the anti-BCMA CAR contains an antigen-binding domain that is an scFv containing a variable heavy (VH) and/or a variable light (VL) region. In some embodiments, the scFv containing a variable heavy (VH) and/or a variable light (VL) region is derived from an antibody described in WO2016090320 or WO2016090327. In some embodiments, the scFv containing a variable heavy (VH) and/or a variable light (VL) region is derived from an antibody described in WO 2019/090003. In some embodiments, the scFv containing a variable heavy (VH) and/or a variable light (VL) region is derived from an antibody described in WO2016094304 or WO2021091978. In some embodiments, the scFv containing a variable heavy (VH) and/or a variable light (VL) region is derived from an antibody described in WO2018133877. In some embodiments, the scFv containing a variable heavy (VH) and/or a variable light (VL) region is derived from an antibody described in WO2019149269. In some embodiments, the anti-BCMA CAR is any as described in WO2019173636 or WO2020051374A. In some embodiments, the anti-BCMA CAR is any as described in WO2018102752. In some embodiments, the anti-BCMA CAR is any as described in WO2020112796 or WO2021173630.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19 (8): 2048-2060, U.S. Pat. No. 9,765,342, WO 2016/090320, WO2016090327, WO2010104949A2, WO2016/0046724, WO2016/014789, WO2016/094304, WO2017/025038, and WO2017173256.

In some embodiments, the anti-BCMA CAR contains an antigen-binding domain, such as an scFv, containing a variable heavy (VH) and/or a variable light (VL) region derived from an antibody described in WO2016094304 or WO2021091978. In some embodiments, the antigen-binding domain is an antibody fragment containing a variable heavy chain (VH) and a variable light chain (VL) region. In some embodiments, the anti-BCMA CAR contains an antigen-binding domain, such as an scFv, containing a variable heavy (VH) and/or a variable light (VL) region derived from an antibody described in WO 2016/090320 or WO2016090327.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723. In some embodiments the antigen-binding domain includes a VH and/or VL derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302).

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from rituximab, such as rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as m971 scFv.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Publication Nos. WO 2016/090329 and WO 2016/090312.

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g., CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g., scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g., scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, e.g., MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three α domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8+ T cells, but in some cases CD4+ T cells. In some embodiments, MHC class II molecules deliver peptides originating in the vesicular system to the cell surface, where they are typically recognized by CD4+ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see, e.g., US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See, e.g., US published application No. US20020150914, US2014/0294841; and Cohen C J. Et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′) 2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6^{th} ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the extracellular portion of the CAR, such as an antibody portion thereof, further includes a spacer, such as a spacer region between the antigen-recognition component, e.g., scFv, and a transmembrane domain. The spacer may be or include at least a portion of an immunoglobulin constant region or variant or modified version thereof, such as a hinge region, e.g., an IgG4 hinge region, and/or a CH1/CL and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. In some embodiments, the spacer has the sequence set forth in SEQ ID NO: 1, and is encoded by the sequence set forth in SEQ ID NO: 2. In some embodiments, the spacer has the sequence set forth in SEQ ID NO: 3. In some embodiments, the spacer has the sequence set forth in SEQ ID NO: 4.

In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some embodiments, the spacer may be or include at least a portion of an immunoglobulin constant region or variant or modified version thereof, such as a hinge region, e.g., an IgG4 hinge region, and/or a CH1/CL and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014/031687. In some embodiments, the spacer has the sequence set forth in SEQ ID NO: 1, and is encoded by the sequence set forth in SEQ ID NO: 2. In some embodiments, the spacer has the sequence set forth in SEQ ID NO: 3. In some embodiments, the spacer has the sequence set forth in SEQ ID NO: 4.

In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

The extracellular ligand binding, such as antigen recognition domain, generally is linked to one or more intracellular signaling components, such as signaling components that mimic activation through an antigen receptor complex, such as a TCR complex, in the case of a CAR, and/or signal via another cell surface receptor. In some embodiments, a transmembrane domain links the extracellular ligand binding and intracellular signaling domains. In some embodiments, the antigen binding component (e.g., antibody) is linked to one or more transmembrane and intracellular signaling regions. In some embodiments, the CAR includes a transmembrane domain fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (e.g., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain and/or region or intracellular signaling domain and/or region of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by at least two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CD8, CD22, CD79a, CD79b, and CD66d. In certain embodiments, ITAM containing primary cytoplasmic signaling sequences include those derived from TCR or CD3 zeta, FcR gamma, or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and ICOS. In some aspects, the same CAR includes both the activating or signaling region and costimulatory components.

In some embodiments, the activating domain is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5 (215) (December 2013), such as a CAR recognizing a different antigen, whereby an activating signal delivered through a CAR recognizing a first antigen is diminished or inhibited by binding of the inhibitory CAR to its ligand, e.g., to reduce off-target effects.

In certain embodiments, the intracellular signaling domain comprises a CD28 transmembrane and signaling domain linked to a CD3 intracellular domain. In some embodiments, the intracellular signaling domain comprises a chimeric CD28 and CD137 co-stimulatory domains, linked to a CD3 intracellular domain.

In some embodiments, the intracellular signaling domain of the CD8⁺ cytotoxic T cells is the same as the intracellular signaling domain of the CD4⁺ helper T cells. In some embodiments, the intracellular signaling domain of the CD8⁺ cytotoxic T cells is different than the intracellular signaling domain of the CD4⁺ helper T cells.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the recombinant receptor(s), e.g., CAR, encoded by nucleic acid(s) within the provided viral vectors further include one or more marker, e.g., for purposes of confirming transduction or engineering of the cell to express the receptor and/or selection and/or targeting of cells expressing molecule(s) encoded by the polynucleotide. In some aspects, such a marker may be encoded by a different nucleic acid or polynucleotide, which also may be introduced during the genetic engineering process, typically via the same method, e.g., transduction by any of the methods provided herein, e.g., via the same vector or type of vector.

In some aspects, the marker, e.g., transduction marker, is a protein and/or is a cell surface molecule. Exemplary markers are truncated variants of a naturally-occurring, e.g., endogenous markers, such as naturally-occurring cell surface molecules. In some aspects, the variants have reduced immunogenicity, reduced trafficking function, and/or reduced signaling function compared to the natural or endogenous cell surface molecule. In some embodiments, the marker is a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A P2A, E2A and/or F2A. See, e.g., WO2014/031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, e.g., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular ligand-binding portion, such as an antigen-binding portion, such as an antibody or fragment thereof and in intracellular domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD32) chain. In some embodiments, the chimeric antigen receptor includes a transmembrane domain linking and/or disposed between the extracellular domain and the intracellular signaling region or domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling region and/or domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region and/or domain comprises an intracellular costimulatory signaling domain of 4-1BB or functional variant thereof, such as a 42-amino acid cytoplasmic domain of a human 4-1BB (Accession No. Q07011.1), or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling region and/or domain comprises a human CD3 chain, optionally a CD3 zeta stimulatory signaling domain or functional variant thereof, such as an 112 AA cytoplasmic domain of isoform 3 of human CD32 (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14, or 15, or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14, or 15.

In some aspects, the spacer contains only a hinge region of an IgG, such as only a hinge of IgG4 or IgG1 such as the hinge only spacer set forth in SEQ ID NO: 1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains, such as set forth in SEQ ID NO: 3. In some embodiments, the spacer is an Ig hinge, e.g., an IgG4 hinge, linked to a CH3 domain only, such as set forth in SEQ ID NO: 4. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of 4-1BB or functional variant thereof; and a signaling portion of CD3 zeta signaling domain or functional variant thereof. In some embodiments, such CAR constructs further includes a T2A ribosomal skip element and/or a tEGFR sequence, e.g., downstream of the CAR

ii. T Cell Receptors (TCRs)

In some embodiments, the recombinant molecule(s) encoded by the nucleic acid(s) is or include a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen. In some embodiments, engineered cells, such as T cells, are provided that express a TCR or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g., cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, e.g., normal TCR libraries. In some embodiments, the TCRs can be amplified from a T cell source of a diseased subject, e.g., diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, the TCR is one that has been cloned from naturally occurring T cells. In some embodiments, a high-affinity T cell clone for a target antigen (e.g., a cancer antigen) is identified and isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17 (12): 1236-1237, and SYFPEITHI (see Schuler et al. (2007) Immunoinformatics Methods in Molecular Biology, 409 (1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17 (12): 1236-1237 2001), and SYFPEITHI (see Schuler et al. SYFPEITHI, Database for Searching and T-Cell Epitope Prediction. In Immunoinformatics Methods in Molecular Biology, vol 409 (1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See, e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see, e.g., International published PCT No. WO 03/020763). In some embodiments, a scTCR is a non-disulfide linked truncated TCR in which heterologous leucine zippers fused to the C-termini thereof facilitate chain association (see, e.g., International published PCT No. WO99/60120). In some embodiments, a scTCR contain a TCRα variable domain covalently linked to a TCRβ variable domain via a peptide linker (see, e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO: 22). In some embodiments, the linker has the sequence GSADDAKKDAAKKDGKS (SEQ ID NO: 23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as a and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-C1, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, the nucleic acid encoding a TCR further includes a marker to confirm transduction or engineering of the cell to express the receptor. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g., lentiviral, vector.

iii. Other Regulatory Elements

In some embodiments of the methods and compositions provided herein, the nucleic acid sequence contained in the viral vector genome encoding an recombinant receptor, such as an antigen receptor, for example a CAR, is operably linked in a functional relationship with other genetic elements, for example transcription regulatory sequences including promoters or enhancers, to regulate expression of the sequence of interest in a particular manner. In certain instances, such transcriptional regulatory sequences are those that are temporally and/or spatially regulated with respect to activity. Expression control elements that can be used for regulating the expression of the components are known and include, but are not limited to, inducible promoters, constitutive promoters, secretion signals, enhancers and other regulatory elements. In some embodiments, the nucleic acid sequence contained in the viral vector genome contain multiple expression control elements that control different encoded components, e.g., different receptor components and/or signaling components, such that the expression, function and/or activity of the recombinant receptor and/or the engineered cell, e.g., cell expressing the engineered receptor, can be regulated, e.g., are inducible, repressible, regulatable and/or user controlled. In some embodiments, one or more vectors can contain one or more nucleic acid sequences that contain one or more expression control elements and/or one or more encoded components, such that the nucleic acid sequences together can regulate the expression, activity and/or function of the encoded components, e.g., recombinant receptor, or the engineered cell.

In some embodiments, the nucleic acid sequence encoding a recombinant receptor, such as an antigen receptor, for example a CAR, is operably linked with internal promoter/enhancer regulatory sequences. The promoters employed may be constitutive, tissue-specific, inducible, and/or useful under the appropriate conditions to direct high level expression of the introduced DNA segment. The promoter may be heterologous or endogenous. In some embodiments, a promoter and/or enhancer is produced synthetically. In some embodiments, a promoter and/or enhancer is produced using recombinant cloning and/or nucleic acid amplification technology.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO: 25 and encoded by the nucleotide sequence set forth in SEQ ID NO: 24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO: 24, or the CD8 alpha signal peptide set forth in SEQ ID NO: 26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see, e.g., U.S. Pat. No. 6,060,273). In some embodiments, transcription units can be engineered as a bicistronic unit containing an IRES (internal ribosome entry site), which allows coexpression of gene products (e.g., encoding the marker and encoding the recombinant receptor) by a message from a single promoter. Alternatively, in some cases, a single promoter may direct expression of an RNA that contains, in a single open reading frame (ORF), two or three genes (e.g., encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g., CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, a E2A or a F2A. Extrinsic marker genes may in some cases be utilized in connection with engineered cell to permit detection or selection of cells and, in some cases, also to promote cell suicide.

Exemplary surrogate markers can include truncated cell surface polypeptides, such as a truncated human epidermal growth factor receptor 2 (tHER2), a truncated epidermal growth factor receptor (EGFRt, exemplary EGFRt sequence set forth in SEQ ID NO: 7 or 16) or a prostate-specific membrane antigen (PSMA) or modified form thereof. EGFRt may contain an epitope recognized by the antibody cetuximab (Erbitux®) or other therapeutic anti-EGFR antibody or binding molecule, which can be used to identify or select cells that have been engineered with the EGFRt construct and a recombinant receptor, such as a chimeric antigen receptor (CAR), and/or to eliminate or separate cells expressing the receptor. See U.S. Pat. No. 8,802,374 and Liu et al., Nature Biotech. 2016 April; 34 (4): 430-434). In some aspects, the marker, e.g., surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g., tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFP), such as super-fold GFP, red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. And Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments a promoter and/or enhancer may be one that is naturally associated with a nucleic acid sequence, as may be obtained by isolating the 5′ non-coding sequences located upstream of the coding segment and/or exon. Alternatively, in some embodiments the coding nucleic acid segment may be positioned under the control of a recombinant and/or heterologous promoter and/or enhancer, which is not normally associated with the coding nucleic acid sequence in the natural setting. For example, exemplary promoters used in recombinant DNA construction include, but are not limited to, the β-lactamase (penicillinase), lactose, tryptophan (trp), RNA polymerase (pol) III promoters including, the human and murine U6 pol III promoters as well as the human and murine H1 RNA pol III promoters; RNA polymerase (pol) II promoters; cytomegalovirus immediate early promoter (pCMV), elongation factor-1 alpha (EF-1 alpha), and the Rous Sarcoma virus long terminal repeat promoter (pRSV) promoter systems. In some embodiments, the promoter may be obtained, for example, from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

In some embodiments, the promoter may be constitutively active. Non-limiting examples of constitutive promoters that may be used include the promoter for ubiquitin (U.S. Pat. No. 5,510,474; WO 98/32869), CMV (Thomsen et al., PNAS 81:659, 1984; U.S. Pat. No. 5,168,062), beta-actin (Gunning et al. 1989 Proc. Natl. Acad. Sci. USA 84:4831-4835) and pgk (see, for example, Adra et al. 1987 Gene 60:65-74; Singer-Sam et al. 1984 Gene 32:409-417; and Dobson et al. 1982 Nucleic Acids Res. 10:2635-2637).

In some embodiments, the promoter may be a tissue specific promoter and/or a target cell-specific promoter. In some embodiments, the promoters may be selected to allow for inducible expression of the sequence of interest. A number of systems for inducible expression are known, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. In some embodiments, the tetracycline-(tet)-regulatable system, which is based on the inhibitory action of the tet repression (tetr) of Escherichia coli on the tet operator sequence (TECO), can be modified for use in mammalian systems and used as a regulatable element for expression cassettes. These systems are well known. (See, Goshen and Badgered, Proc. Natl. Acad. Sci. USA 89:5547-51 (1992), Shockett et al., Proc. Natl. Acad. Sci. USA 92:6522-26 (1996), Lindemann et al., Mol. Med. 3:466-76 (1997)).

A combination of promoters may also be used to obtain the desired expression of the gene of interest. The artisan of ordinary skill will be able to select a promoter based on the desired expression pattern of the gene in the organism or the target cell of interest.

In some embodiments, an enhancer may also be present in the viral construct to increase expression of the gene of interest. Enhancers are typically cis-acting nucleic acid elements, usually about 10 to 300 by in length, that act on a promoter to increase its transcription. Many enhancers in viral genomes, such as HIV or CMV are known. For example, the CMV enhancer (Boshart et al. Cell, 41:521, 1985) can be used. Other examples include, for example, the SV40 enhancer on the late side of the replication origin (bp 100-270), the cytomegalovirus early promoter enhancer, the polyoma enhancer on the late side of the replication origin, and adenovirus enhancers. In some cases, an enhancer is from a mammalian gene, such as an enhancer from a globin, elastase, albumin, alpha-fetoprotein or insulin). An enhancer can be used in combination with a heterologous promoter. The enhancer may be spliced into the vector at a position 5′ or 3′ to the polynucleotide sequence encoding the gene of interest, but is generally located at a site 5′ from the promoter. One of ordinary skill in the art will be able to select the appropriate enhancer based on the desired expression pattern.

The viral vector genome may also contain additional genetic elements. The types of elements that can be included in the constructs are not limited in any way and can be chosen by one with skill in the art.

For example, a signal that facilitates nuclear entry of the viral genome in the target cell may be included. An example of such a signal is the HIV-1 flap signal (in some cases referred to as the flap sequence). In addition, the vector genome may contain one or more genetic elements designed to enhance expression of the gene of interest. In some embodiments, the genome contains a post-transcriptional regulatory element (PRE) or modified form thereof that exhibits post-transcriptional activity. For example, in some embodiments, a woodchuck hepatitis virus posts-transcriptional responsive element (WPRE) may be placed into the construct (Zufferey et al. 1999. J. Virol. 74:3668-3681; Deglon et al. 2000. Hum. Gene Ther. 11:179-190). In some embodiments, the vector genome lacks a flap sequence and/or lacks a WPRE. In some embodiments, the vector genome contains a mutated or defective flap sequence and/or WPRE.

In some instances, more than one open reading frame encoding separate heterologous proteins can be included. For example, in some embodiments, if a reporter and/or detectable and/or selectable gene is included in the expression construct, an internal ribosomal entry site (IRES) sequence can be included. Typically, the additional genetic elements are operably linked with and controlled by an independent promoter/enhancer. The additional genetic element can be a reporter gene, a selectable marker or other desired gene.

In some embodiments, other various regulatory elements can include a transcription initiation region and/or a termination region. Expression vectors may also contain sequences for the termination of transcription and for stabilizing the mRNA. Such sequences are known and are often found naturally in the 5′ and, occasionally 3′, untranslated regions of eukaryotic or viral DNAs or cDNAs. Examples of transcription termination region include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

In some embodiments, regulatory elements can include regulatory elements and/or systems that allow regulatable expression and/or activity of the recombinant receptor, e.g., CAR. In some embodiments, regulatable expression and/or activity is achieved by configuring the recombinant receptor to contain or be controlled by particular regulatory elements and/or systems. In some embodiments, one or more additional receptors can be used in an expression regulation systems. In some embodiments, expression regulation systems can include systems that require exposure to or binding of a specific ligand that can regulate the expression and/or activity of the recombinant receptor. In some embodiments, regulated expression of the recombinant receptor, e.g., CAR, is achieved a regulatable transcription factor release system, e.g., a modified Notch signaling system (see, e.g., Roybal et al., Cell (2016) 164:770-779; Morsut et al., Cell (2016) 164:780-791). In some embodiments, regulation of activity of the recombinant receptor is achieved by administration of an additional agent that can induce conformational changes and/or multimerization of polypeptides, e.g., the recombinant receptor. In some embodiments, the additional agent is a chemical inducer (see, e.g., U.S. Patent Publication No. 2016/0046700, Clackson et al. (1998) Proc Natl Acad Sci USA. 95 (18): 10437-42; Spencer et al. (1993) Science 262 (5136): 1019-24; Farrar et al. (1996) Nature 383 (6596): 178-81; Miyamoto et al. (2012) Nature Chemical Biology 8 (5): 465-70; Erhart et al. (2013) Chemistry and Biology 20 (4): 549-57).

### B. Preparation of Viral Vector Particles

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, e.g., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, e.g., a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

### V. COMPOSITIONS, FORMULATIONS, AND METHODS OF ADMINISTRATION

Also provided are compositions containing the transduced populations of cells expressing an engineered receptor (e.g., engineered antigen receptor), such as a chimeric antigen receptor (CAR) or a T cell receptor (TCR), and compositions containing the engineered cells, including pharmaceutical compositions and formulations. Also provided are methods of using and uses of the compositions, such as in the treatment of diseases, conditions, and disorders in which the antigen is expressed, or in detection, diagnostic, and prognostic methods.

Also provided are compositions containing the populations of cells that have been enriched for viable cells, which can be transduced population of cells that have been enriched for viable cells. Also provided herein are compositions containing the populations of cells that have been debeaded, which can be transduced population of cells that have been debeaded. In some embodiments, the transduced populations of cells express an engineered receptor (e.g., engineered antigen receptor), such as a chimeric antigen receptor (CAR) or a T cell receptor (TCR). In some embodiments, the compositions include pharmaceutical compositions and formulations. Also provided are methods of using and uses of the compositions, such as in the treatment of diseases, conditions, and disorders in which the antigen is expressed, or in detection, diagnostic, and prognostic methods

### A. Compositions and Formulations

In some embodiments, a dose of cells comprising cells engineered with a recombinant antigen receptor, e.g., CAR or TCR, is provided as a composition or formulation, such as a pharmaceutical composition or formulation. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16^{th} edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21^{st} ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### B. Dosages and Administration

The cells or compositions are administered at a dose to result in a therapeutically effective amount of recombinant receptor (e.g., CAR)-expressing cells in vivo for treating the disease or condition. For the prevention or treatment of disease, the appropriate dosage may depend on the type of disease to be treated, the type of cells or recombinant receptors, administration of other drugs or agents in combination, such as those that boost, augment or enhance cell expansion, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered and other drugs or agents being administered in combination, such as concurrently.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

In some embodiments, the cells or compositions are administered in an amount that is effective to treat or prevent the disease or condition, such as a therapeutically effective or prophylactically effective amount. Thus, in some embodiments, the methods of administration include administration of the cells and compositions at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

In some embodiments, a therapeutically effective amount of cells are administered to the subject. In some embodiments, a sub-optimal dose of cells are administered to a subject, such as in certain cases in which the cells are administered under conditions for in vivo expansion of cells.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., less than 0.5 million cells, less than 1 million cells, about 0.1 million cells, about 0.2 million cells, about 0.3 million cells, about 0.4 million cells, about 0.5 million cells, about 1 million cells, about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg.

In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g., CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8+ and CD4+ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g., consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. For example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, a relatively lower dose of cells, such as a sub-optimal dose of cells or a dose of cells lower than a therapeutically effective amount, may be administered, which, upon in vivo stimulation (e.g., by an endogenous antigen or exogenous agent) can lead to a boost, such as an increase or expansion, in the number of engineered cells present in the subject. In any of such embodiments, expansion and/or activation of cells can occur with exposure to an antigen in vivo, e.g., expansion of the engineered cells in the body of the subject after administration of the cells. In some embodiments, the extent, degree or magnitude of in vivo expansion can be further augmented, boosted or enhanced by various methods that are able to modulate, e.g., increase, expansion, proliferation, survival and/or efficacy of the administered cells, e.g., recombinant receptor expressing cells.

Once the cells are administered to the subject (e.g., human), the biological activity of the cell populations in some aspects is measured by any of a number of known methods. Parameters to assess include specific binding of the cells to antigen, in vivo, e.g., by imaging, or ex vivo, e.g., by ELISA or flow cytometry. In certain embodiments, the ability of the cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

### VI. ARTICLES OF MANUFACTURE AND KITS

In some embodiments, also provided are systems, apparatuses, and kits useful in performing the provided methods. In some embodiments, provided are articles of manufacture, such as kits or devices, containing viral particles and/or cells, and optionally instructions for use. In some embodiments, the kits can be used in methods for transducing cells, such as in accord with preparing genetically engineered cells for adoptive cell therapy. In some embodiments, the kits can be used in methods for enriching for viable cells, such as in transduced cells. In some embodiments, the kits can be used for debeading cells.

In some embodiments, the articles of manufacture include one or more containers, typically a plurality of containers, packaging material, and a label or package insert on or associated with the container or containers and/or packaging, generally including instructions for transduction of cells, such as transduction of cells from a subject. Alternatively, the instructions can be for the enrichment of viable cells or the debeading of cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, or bottles. The articles of manufacture can include a needle or other injection device so as to facilitate dispensing of the materials. Typically, the packaging is non-reactive with the compositions contained therein.

In some embodiments, the viral particles and the cells are packaged separately. In some embodiments, each container can have a single compartment. In some embodiments, a container containing the viral particles is one that is amenable to addition of the cells by the user, for example through an opening in the compartment, or vice versa. Any container or other article of manufacture that is amenable to having a defining space for containment of the viral particles and/or the cells and that is amenable to simple manipulation to permit addition of the final components necessary for mixture to produce an input composition containing viral particles associated with the cells is contemplated. In some embodiments, the cells are added to the viral particles prior to loading of the cell composition and the viral particle into the centrifuge.

In some embodiments, such materials are packaged separately in the same container, for example, such that the components can be mixed or combined in the container. In some aspects, examples of such containers include those that have an enclosed, defined space that contains the viral particles, and a separate enclosed, defined space containing the cells such that the two spaces are separated by a readily removable membrane which, upon removal, permits the components to mix. Any container or other article of manufacture is contemplated in which the components can be kept separate. In some embodiments, an article of manufacture can contain each component in adjacent compartments separated by a dividing member, such as a membrane, that, upon compression of the article of manufacture, ruptures, permitting separated components to mix. For suitable embodiments, see, e.g, containers described in U.S. Pat. Nos. 3,539,794 and 5,171,081.

### VII. DEFINITIONS

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vector particles, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a composition refers to any mixture of two or more products, substances, or compounds, including cells. It may be a solution, a suspension, liquid, powder, a paste, aqueous, non-aqueous, or any combination thereof.

As used herein, “enriching” when referring to one or more particular cell types or cell populations, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for a cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for a cell known to be positive for the marker, and/or at a level substantially similar as compared to that for a cell known to be negative for the marker.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

As used herein, “reverse” or “counterflow” centrifugation refers to a technique whereby the settling rate of particles in a fluid (e.g., a supporting media) under centrifugal acceleration is counteracted by a flow of the supporting media.

As used herein, “continuous” flow centrifugation, as opposed to “dead end” or “batch” centrifugation, refers to a technique whereby large volumes or material can be centrifuged, including at high centrifugal forces, without requiring frequent stopping and starting the rotor, or frequent filling and decanting centrifuge tubes. A continuous flow system can function on a continuous flow basis. An example of dead-end centrifugation is a swing-bucket rotor centrifugation.

As used herein, a control refers to a sample that is substantially identical to the test sample, except that it is not treated with a test parameter, or, if it is a plasma sample, it can be from a normal volunteer not affected with the condition of interest. A control also can be an internal control.

### VIII. EXEMPLARY EMBODIMENTS

Among the embodiments provided herein are:

1. A method for producing a composition of genetically engineered T cells, the method comprising:
(a) applying a first centrifugal force and a first flow rate to a cell composition comprising T cells in a conical fluid enclosure of a centrifuge system to produce a fluidized bed of cells;
(b) loading a viral vector particle into the conical fluid enclosure, thereby generating an input composition comprising the cell composition and the viral vector particle; and
(c) applying a second centrifugal force and a second flow rate to the input composition, wherein the second centrifugal force and second flow rate recirculate the viral vector particle in a fluid path of the centrifuge system, thereby generating genetically engineered T cells.

2. The method of embodiment 1, wherein the loading of the viral vector particle is carried out during at least a portion of the applying in (a) and/or is carried out during at least a portion of the applying in (c).

3. A method for producing a composition of genetically engineered T cells, the method comprising:
(a) applying a first centrifugal force and a first flow rate to an input composition comprising (i) a viral vector particle and (ii) a cell composition comprising T cells in a conical fluid enclosure of a centrifuge system to produce a fluidized bed of cells; and
(b) applying a second centrifugal force and a second flow rate to the input composition in the conical fluid enclosure, wherein the second centrifugal force and second flow rate recirculate the viral vector particle in a fluid path of the centrifuge system, thereby generating genetically engineered T cells.

4. The method of embodiment 3, further comprising loading the cell composition and the viral vector particle into the conical fluid enclosure, thereby generating the input composition, wherein the loading of the cell composition is before, during, and/or after the loading of the viral vector particle.

5. The method of embodiment 4, wherein the loading of the cell composition and/or the loading of the viral vector particle is performed prior to and/or during the applying in (a).

6. The method of any of embodiments 1-5, wherein the centrifuge system is a continuous counterflow centrifuge system.

7. The method of any one of embodiments 1-6, further comprising applying a third centrifugal force and a third flow rate to the genetically engineered T cells in the conical fluid enclosure of the centrifuge system to produce an output composition comprising the genetically engineered T cells.

8. The method of embodiment 7, wherein the percentage of viable T cells in the output composition is greater than the percentage of viable T cells in the input composition, optionally at least about 5% greater, at least about 10% greater, at least about 15% greater, at least about 20% greater, or at least about 25% greater.

9. The method of embodiment 7 or embodiment 8, wherein at least or at least about 5%, at least or at least about 10%, at least or at least about 15%, at least or at least about 20%, at least or at least about 25%, or at least or at least about 30% of the T cells in the output composition are transduced with the viral vector particle.

10. The method of any one of embodiments 1-9, wherein (i) the first centrifugal force is between about 2,000 G and about 4,000 G; and (ii) the first flow rate is between about 5 mL/min and about 15 mL/min, optionally wherein the first centrifugal force and the first flow rate are applied to the cell composition or the input composition for about 15 seconds, about 30 seconds, about 45 seconds, or about 60 seconds.

11. The method of any one of embodiments 1-10, wherein (i) the second centrifugal force is between about 500 G and about 1,500 G; and (ii) the second flow rate is between about 25 mL/min and about 30 mL/min.

12. The method of any one of embodiments 11, wherein the ratio of the first centrifugal force to the first flow rate is between about 200 and about 400.

13. The method of any one of embodiments 1-12, wherein the ratio of the first centrifugal force to the first flow rate is about 300.

14. The method of any one of embodiments 1-13, wherein the first centrifugal force is about 3,000 G and the first flow rate is about 10 mL/min.

15. The method of any one of embodiments 1-14, wherein the ratio of the second centrifugal force to the second flow rate is between about 20 and about 100, between about 25 and about 85, or between about 30 and about 65.

16. The method of any one of embodiments 1-15, wherein the ratio of the second centrifugal force to the second flow rate is about 35.

17. The method of any one of embodiments 1-16, wherein the second centrifugal force is about 1,000 G and the second flow rate is about 28.5 mL/min.

18. The method of any one of embodiments 1-15, wherein the ratio of the second centrifugal force to the second flow rate is about 62.5.

19. The method of any one of embodiments 1-17, wherein the second centrifugal force and the second flow rate are applied to the input composition for at least about 15 minutes, at least about 30 minutes, at least about 45 minutes, at least about 60 minutes, at least about 75 minutes, or at least about 90 minutes.

20. The method of any one of embodiments 7-19, wherein (i) the third centrifugal force is between about 2,000 G and about 3,000 G; and (ii) the third flow rate is between about 15 mL/min and about 25 mL/min.

21. The method of any one of embodiments 7-20, wherein the ratio of the third centrifugal force to the third flow rate is between about 100 and about 150, optionally wherein the ratio of the third centrifugal force to the third flow rate is about 125.

22. The method of any one of embodiments 7-21, wherein the third centrifugal force is about 2,500 G and the third flow rate is about 20 mL/min.

23. The method of any one of embodiments 7-22, wherein prior to the applying the third centrifugal force and the third flow rate, the method comprises subjecting the genetically engineered T cells to one or more washing steps, optionally wherein the one or more washing steps comprises media exchange.

24. The method of any one of embodiments 1-23, wherein the method comprises incubating the T cells of the cell composition under stimulating conditions prior to the applying in (a) and/or the T cells of the cell compositions are incubated under stimulating conditions prior to the applying in (a).

25. The method of embodiment 24, wherein the stimulating conditions comprise the presence of a stimulatory reagent that is capable of activating one or more intracellular signaling domains of one or more components of a TCR complex and one or more intracellular signaling domains of one or more costimulatory molecules.

26. The method of embodiment 25, wherein the stimulatory reagent comprises (i) a primary agent that specifically binds to a member of a TCR complex, optionally that specifically binds to CD3; and (ii) a secondary agent that specifically binds to a T cell costimulatory molecule, optionally wherein the costimulatory molecule is selected from CD28, CD137 (4-1-BB), OX40 or ICOS.

27. The method of embodiment 26, wherein at least one of the primary and secondary agents comprises an antibody or an antigen-binding fragment thereof.

28. The method of embodiment 26 or embodiment 27, wherein the primary agent is an anti-CD3 antibody or an antigen-binding fragment thereof and the secondary agent is an anti-CD28 antibody or an antigen-binding fragment thereof.

29. The method of any one of embodiments 26-28, wherein the primary agent and the secondary agent are each present or attached on the surface of a solid support, optionally wherein the solid support is or comprises a bead, further optionally wherein the solid support is a paramagnetic bead with surface attached anti-CD3 and anti-CD28 antibodies.

30. The method of any one of embodiments 26-28, wherein the primary agent and the secondary agent are reversibly bound on the surface of an oligomeric particle reagent comprising a plurality of streptavidin molecules or streptavidin mutein molecules.

31. The method of embodiment 30, wherein the streptavidin molecules or the streptavidin mutein molecules bind to or are capable of binding to biotin, avidin, a biotin analog or a biotin mutein, an avidin analog or an avidin mutein and/or a biologically active fragment thereof.

32. The method of any one of embodiments 26-30, wherein the primary agent comprises an anti-CD3 Fab and wherein the secondary agent comprises an anti-CD28 Fab.

33. The method of any one of embodiments 24-32, wherein the stimulating conditions comprise the presence of one or more recombinant cytokines.

34. The method of any one of embodiments 24-33, wherein the stimulating conditions comprise the presence of one or more of recombinant IL-2, IL-7 and IL-15.

35. The method of any one of embodiments 7-34, wherein the method comprises collecting the output composition and/or the output composition is collected.

36. The method of embodiment 35, wherein the method comprises incubating the genetically engineered T cells of the collected output composition and/or the genetically engineered T cells of the collected output composition are incubated.

37. The method of embodiment 35 or embodiment 36, wherein the genetically engineered T cells of the collected output composition are incubated immediately following the collecting for at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days.

38. The method of any one of embodiments 35-37, wherein the percentage of viable T cells in the collected output composition about 1 day after, about 2 days after, about 3 days after, about 4 days after, about 5 days after, about 6 days after, about 7 days after, about 8 days after, about 9 days after, or about 10 days after collection is greater than the percentage of viable T cells in the input composition.

39. The method of any one of embodiments 35-38, wherein the percentage of viable T cells in the collected output composition about 1 day after collection is greater than the percentage of viable T cells in the input composition.

40. The method of any one of embodiments 35-39, wherein the percentage of viable T cells in the collected output composition about 5 days after collection is greater than the percentage of viable T cells in the input composition.

41. The method of any one of embodiments 35-40, wherein the method comprises cryopreserving the collected output composition and/or the collected output composition is cryopreserved, thereby generating a cryopreserved composition.

42. The method of embodiment 41, wherein the cryopreserved composition is thawed to produce a thawed composition, and the percentage of viable T cells in the thawed composition is greater than the percentage of viable T cells in the input composition, optionally at least about 5% greater, at least about 10% greater, at least about 15% greater, at least about 20% greater, at least about 25% greater, or at least about 30% greater.

43. The method of any one of embodiments 1-42, wherein the input composition comprises T cells having an average diameter of greater than or greater than about 6 microns, greater than or greater than about 6 microns, greater than or greater than about 7 microns, greater than or greater than about 8 microns, greater or greater than about 9 microns, greater or greater than about 10 microns, or greater or greater than about 11 microns.

44. The method of any one of embodiments 1-43, wherein the input composition comprises between about 1×10⁶ total T cells and about 2×10⁹ total T cells.

45. The method of any one of embodiments 1-44, wherein the input composition comprises at least about 1×10⁸ total T cells, at least about 2×10⁸ total T cells, at least about 3×10⁸ total T cells, at least about 4×10⁸ total T cells, at least about 5×10⁸ total T cells, at least about 6×10⁸ total T cells, at least about 7×10⁸ total T cells, at least about 8×10⁸ total T cells, at least about 7×10⁸ total T cells, at least about 8×10⁸ total T cells, at least about 9×10⁸ total T cells, at least about 1×10⁹ total T cells, at least about 1.25×10⁹ total T cells, at least about 1.50×10⁹ total T cells, or at least about 1.75×10⁹ total T cells.

46. The method of any one of embodiments 1-45, wherein the volume of the input composition is between about 5 ml and about 20,000 ml, between about 10 mL and about 2,000 mL. between about 15 mL and about 1,000 mL, between about 20 mL and about 500 mL, between about 25 mL and about 100 mL, or between about 30 mL and about 60 mL.

47. The method of any one of embodiments 1-46, wherein the volume of the input composition is between about 30 mL and about 60 mL.

48. The method of any one of embodiments 7-47, wherein the volume of the output composition is between about 2.5 mL and about 60 mL, between about 5 mL and about 40 mL, or between about 10 mL and about 20 mL.

49. The method of any one of embodiments 7-48, wherein the volume of the output composition is about 5 mL, about 10 mL, about 15 mL, about 20 mL, about 25 mL, about 30 mL, about 35 mL, about 40 mL, about 45 mL, about 50 mL, about 55 mL, or about 60 mL.

50. A method of enriching a cell composition for viable cells, the method comprising:
(a) applying a first centrifugal force and a first flow rate to a cell composition comprising T cells in a conical fluid enclosure of a centrifuge system to produce a fluidized bed of cells, wherein the cell composition comprises viable and non-viable T cells, wherein (i) the first centrifugal force is between about 2,000 G and about 4,000 G; and (ii) the first flow rate is between about 5 mL/min and about 15 mL/min; and
(b) applying a second centrifugal force and a second flow rate to the cell composition, wherein the second centrifugal force and second flow rate recirculate cells of the input composition in a fluid path of the centrifuge system, thereby generating an enriched composition having a higher percentage of viable T cells than the percentage of viable T cells in the cell composition, wherein (i) the second centrifugal force is between about 500 G and about 1,500 G; and (ii) the second flow rate is between about 25 mL/min and about 30 mL/min.

51. The method of embodiment 50, wherein the method comprises loading the cell composition into the centrifuge system, wherein the loading is performed prior to and/or during at least a portion of the applying in (a).

52. The method of embodiment 50 or embodiment 51, wherein the centrifuge system is a continuous counterflow centrifuge system.

53. The method of any one of embodiments 50-52, wherein prior to the applying in (a), the method comprises contacting the T cells of the cell composition with a viral vector particle, thereby producing genetically engineered T cells and/or the T cells of the cell composition have been contacted with a viral vector particle, thereby producing genetically engineered T cells.

54. The method of any one of embodiments 50-53, wherein the percentage of viable T cells in the enriched composition is at least about 10% greater, at least about 20% greater, at least about 30% greater, at least about 40% greater, at least about 50% greater, or at least about 60% greater than the percentage of viable T cells in the cell composition.

55. The method of any one of embodiments 50-54, comprising (c) applying a third centrifugal force and a third flow rate to the enriched composition in the conical fluid enclosure of the centrifuge system to collect the enriched composition, wherein (i) the third centrifugal force is between about 2,000 G and about 3,000 G; and (ii) the third flow rate is between about 15 mL/min and about 25 mL/min.

56. The method of embodiment 55, wherein prior to the applying the third centrifugal force and the third flow rate, the method comprises subjecting the enriched composition to one or more washing steps, optionally wherein the one or more washing steps comprises media exchange.

57. The method of any one of embodiments 23-49 and 56, wherein the one or more washing steps is carried out at the second centrifugal force and the second flow rate.

58. The method of any one of embodiments 1-57, wherein the cell composition comprises activated T cells.

59. The method of any one of embodiments 50-58, wherein the method comprises cryopreserving the collected enriched composition and/or the collected enriched composition is cryopreserved, thereby generating a cryopreserved enriched composition.

60. The method of embodiment 59, wherein the cryopreserved enriched composition is thawed to produce a thawed enriched composition, and the percentage of viable T cells in the thawed enriched composition is greater than the percentage of viable T cells in the cell composition, optionally at least about 5% greater, at least about 10% greater, at least about 15% greater, at least about 20% greater, at least about 25% greater, or at least about 30% greater.

61. The method of any one of embodiments 1-60, wherein one or more steps of the method is automated, optionally wherein the one or more steps of the method is automated by the centrifuge system or a component thereof.

62. The method of any one of embodiments 1-61, wherein the viral vector particle comprises a heterologous nucleic acid encoding a recombinant molecule.

63. The method of embodiment 62, wherein the recombinant molecule is a chemokine, a chemokine receptor, a cytokine, a cytokine receptor, an antigen receptor (e.g., a CAR or a TCR), or a combination thereof.

64. The method of embodiment 62 or embodiment 63, wherein the recombinant molecule is an antigen receptor.

65. The method of embodiment 64, wherein the antigen receptor is a transgenic T cell receptor (TCR).

66. The method of embodiment 65, wherein the antigen receptor is a chimeric antigen receptor (CAR).

67. The method of embodiment 66, wherein the chimeric antigen receptor (CAR) comprises an extracellular antigen-recognition domain that specifically binds to a target antigen and an intracellular signaling domain comprising an ITAM.

68. The method of embodiment 67, wherein the intracellular signaling domain comprises an intracellular domain of a CD3-zeta (CD32) chain.

69. The method of embodiment 67 or embodiment 68, wherein the CAR further comprises a transmembrane domain linking the extracellular domain and the intracellular signaling domain.

70. The method of embodiment 69, wherein the transmembrane domain comprises a transmembrane portion of CD28.

71. The method of any one of embodiments 67-70, wherein the intracellular signaling domain further comprises an intracellular signaling domain of a T cell costimulatory molecule.

72. The method of embodiment 71, wherein the T cell costimulatory molecule is selected from the group consisting of CD28 and 4-1BB.

73. The method of any one of embodiments 66-72, wherein the CAR is recombinantly expressed.

74. The method of any one of embodiments 66-73, wherein the CAR is expressed from a vector, optionally a γ-retroviral vector or a lentiviral vector.

75. The method of any one of embodiments 66-74, wherein the CAR is expressed from a lentiviral vector.

76. The method of any one of embodiments 64-75, wherein the antigen receptor specifically binds to an antigen associated with a disease or a condition.

77. The method of embodiment 76, wherein the disease or condition is a cancer, an autoimmune disease or disorder, and/or an infectious disease. 78. The method of embodiment 76 or embodiment 77, wherein the disease or condition is a cancer.

79. The method of any one of embodiments 1-78, wherein the T cells are primary T cells, optionally from a human subject.

80. A composition comprising genetically engineered T cells produced by the method of any one of embodiments 1-79.

81. The composition of embodiment 80, wherein the composition comprises between about 1.0×10⁶ CAR-expressing T cells and 2.0×10⁹ CAR-expressing T cells.

82. The composition of embodiment 80 or 81, further comprising a pharmaceutically acceptable carrier.

83. The composition of embodiment 80 or embodiment 81, further comprising a cryoprotectant.

84. A method of treating a subject having a disease or disorder, the method comprising administering the composition of any one of embodiments 80-82 to the subject.

85. The method of embodiment 84, wherein the genetically engineered T cells express an antigen receptor specifically binds to an antigen associated with the disease or disorder.

### IX. EXAMPLES

The following examples are included for illustrative purposes only and are not intended to limit the scope of the invention.

### Example 1 Enrichment of Viable Cells During Transduction by Continuous Counterflow Centrifugation

A. Enrichment of Viable Cells During Transduction Separate compositions of CD4+ and CD8+ cells were selected by immunoaffinity-based enrichment from isolated PBMCs from leukapheresis samples from four human donors. The selected cell compositions were cryopreserved, subsequently thawed, and cultured at a 1:1 ratio of viable CD4+ to viable CD8+ cells. The combined CD4+ and CD8+ cell composition was then incubated with anti-CD3/anti-CD28 Fab conjugated oligomeric streptavidin mutein reagents (see, e.g., WO2018197949 and Poltorak et al. (2020) Sci Rep 10, 17832) in media containing recombinant IL-2, IL-7, and IL-15 to activate the T cells for 18-30 hours.

Following activation, the T cell composition was subjected to a continuous counterflow elutriation (“CCE”)-based method of centrifugation in a continuous counterflow centrifuge system (CTS Rotea™ Counterflow Centrifugation System) in the presence of lentiviral vector particles encoding an anti-CD19 chimeric antigen receptor (CAR), to transduce the T cells with the vector (“input composition”). The CAR contained an anti-CD19 scFv derived from a murine antibody (variable region derived from FMC63, an immunoglobulin-derived spacer, a transmembrane domain derived from CD28, a costimulatory region derived from 4-1BB, and a CD3-zeta intracellular signaling domain). The expression construct in the viral vector further contained sequences encoding a truncated receptor, which was separated from the CAR sequence by a T2A ribosome skip sequence, and served as a surrogate marker for CAR expression.

To begin, the T cell composition was generated to contain 1-2 million cells/mL in a total media volume of 200 mL, and was loaded into the conical enclosure portion of the system via a narrow cannula running along the length of the conical enclosure portion. For loading, the T cell composition entered the cannula at the wide end of the conical enclosure portion and exited the cannula at the tip of the conical enclosure portion. The T cell composition was initially subjected to a centrifugal force of 3,000×g and a flow rate of 10 mL/minute, yielding a centrifugal force to flow rate ratio of 3,000:10 (G/FR of 300) to form a fluidized bed of cells. Non-viable cells tended to aggregate toward the outer edges of the tip of the conical enclosure portion. After establishment of the fluidized cell bed, a 10 mL solution containing the lentiviral vector particles was loaded into the conical enclosure portion via the cannula of the continuous counterflow centrifuge system, with maintenance of the centrifugal force and flow rate, to establish a fluidized bed of cells and vector particles.

After the fluidized bed of cells and vector particles was established, the flow rate was maintained at 10 mL/min, but at a centrifugal force of 625×g, yielding a G/FR of 62.5. These conditions allowed larger, sticky non-viable T cell aggregates to continue to aggregate as a pellet at the conical tip of the conical enclosure portion (“waste” fraction), while fluidized T cells (either viable or non-viable) and vector particles were forced toward the center of the conical enclosure portion and allowed to exit (elutriate from) the wide end of the conical enclosure portion and flow into a collection chamber through a fluid path as an “output” fraction. The output fraction was recirculated through the conical enclosure portion of the continuous counterflow centrifuge system for a total of 30 minutes, until the cell composition was redirected and collected as a final transduced cell “product” fraction.

After recirculation, the output fraction was redirected and collected as a final viable cell “product” fraction at a centrifugal force of 2,500×g and a flow rate of 20 mL/min, yielding a G/FR of 125. The transduced “product” fraction was collected via the cannula, with collected cells entering the cannula at the tip of the conical enclosure portion and exiting the cannula at the wide end of the conical enclosure portion. The transduced “product” fraction was resuspended in media at a concentration of 0.75×10⁶ cells/mL and incubated for 24 hours at 37 degrees Celsius to allow for integration of the viral vector. The number of viable and non-viable cells in the various fractions were determined by a fluorescent microscopy-based cell enumeration instrument.

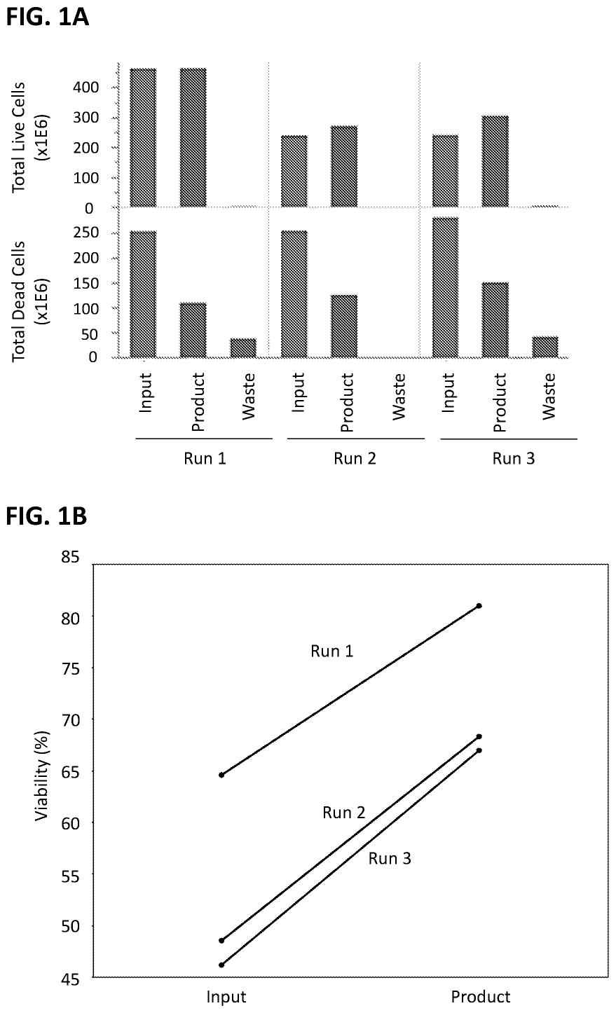

Results from three different runs are shown in FIGS. 1A and 1B. The exemplary centrifugation method of transduction removed an average of approximately 50% of non-viable cells (FIG. 1A, bottom panel) and increased the viable cell yield on average by approximately 20% (FIG. 1B). These results demonstrate that a CCE-based method of transduction can enrich for and increase the percentage of viable cells in a sample.

### B. Transduction Efficiency and Cell Viability Following Transduction

Primary human T cell compositions were generated from leukapheresis samples from human adult donors and subjected to the CCE-based method of centrifugation in the presence of a lentiviral vector encoding an anti-CD19 CAR to transduce the T cells with the vector (“input” composition), as described in Section A. Transduction efficiency and cell viability of the centrifuged cell composition was assessed immediately following transduction, as well as at multiple time points thereafter.

As a control, T cells were transduced with the lentiviral vector by a scaled-down method, either with or without spinoculation. For the spinoculation method, the T cell composition was generated to contain 2×10⁷ viable cells/mL and centrifuged at 693 g in a substantially rigid cylindrical centrifugal chamber for 30 minutes. The “no spin” control sample was incubated under the same conditions, but without centrifugation. The vector titer was 1.11 μL per million cells for all samples. The control compositions were also incubated following transduction to allow for integration of the viral vector.

Following incubation, the transduced product fractions were collected and washed by an automated method of dead-end centrifugation and buffer exchange. Transduction efficiency was determined based on the percentage of CD3+ T cells with surface expression of the CAR in the transduced cell compositions. Viability of the transduced cell compositions was assessed as described in Section A.

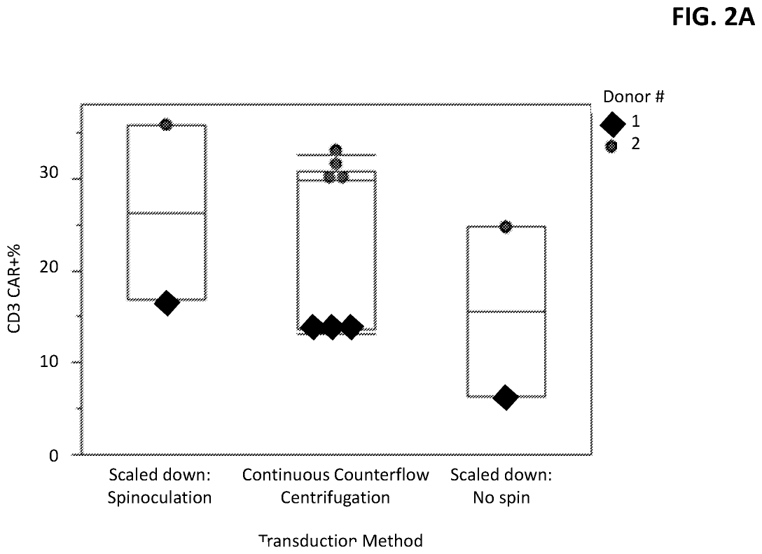

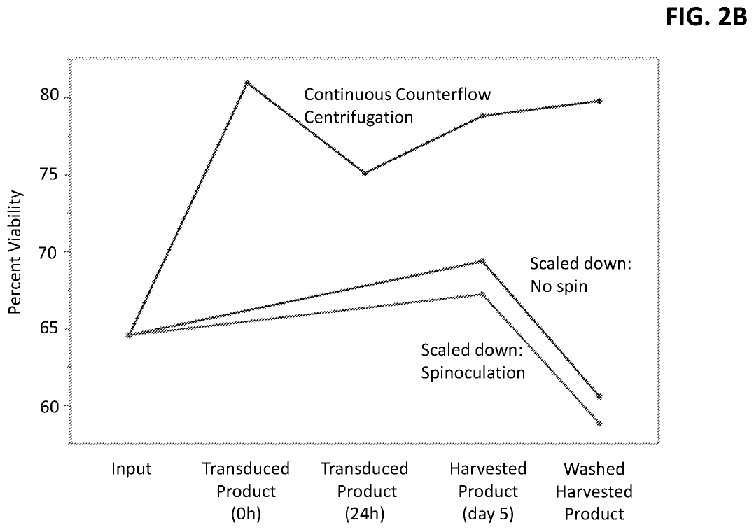

As shown in FIG. 2A for transduced cell compositions produced from cells from two donors, the CCE-based method resulted in improved transduction efficiency, relative to the no-spin control method. The transduction efficiency of the CCE method was lower than that achieved with the spinoculation method.

As shown in FIG. 2B, the viability of the T cells increased from 65 percent to 81 percent immediately following continuous counterflow centrifugation with the vector (“input” vs. “Oh”). At both 24 hours (“24 h”) and 5 days (“day 5”) post-transduction, the transduced T cells continued to exhibit increased cell viability as compared to their pre-transduction viability. By contrast, cell viability was not significantly changed from pre-transduction viability levels in the control samples.

### C. Effect of Transduction Volume on Cell Viability

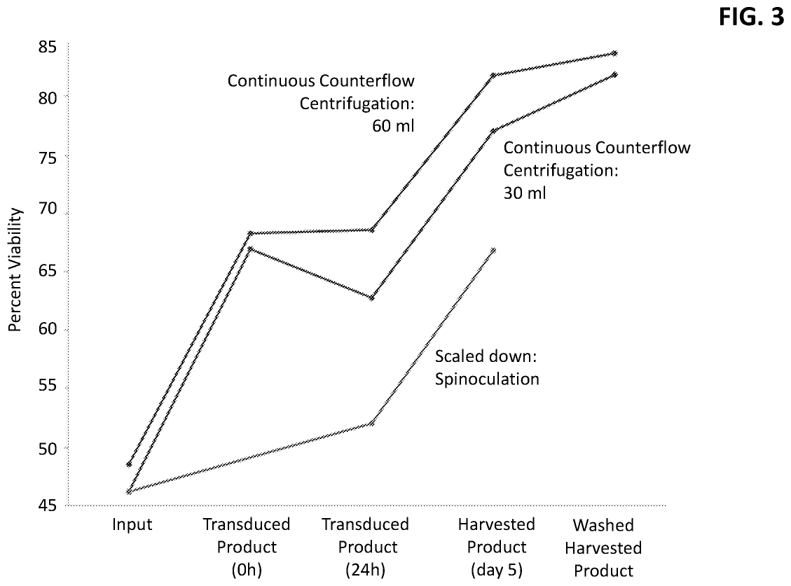

In a related experiment, T cells were transduced by the same methods in a continuous counterflow centrifuge system by the CCE method with 1.11 μL of vector per million cells in a total volume of either 30 mL or 60 mL. As a control, T cells were subjected to the scaled-down spinoculation method. The viability of both the 30 mL and 60 mL cell and vector compositions subjected to the CCE method increased comparably (FIG. 3), indicating that composition volume did not have a significant effect on the viability of transduced cells.

The results are consistent with an observation that a continuous counterflow centrifuge system can be used to simultaneously transduce a T cell composition with a viral vector particle and enrich the composition for viable T cells.

### Example 2 Enrichment of Transduced Viable Cells by Continuous Counterflow Centrifugation Vs. Alternative Automated Centrifugation

T cells compositions were generated from 11 human donors and activated as described in Example 1. Following activation, the cells were resuspended in media containing recombinant IL-2, IL-7, and IL-15 and transduced with a lentiviral vector encoding an anti-CD19 CAR by spinoculation at 693 g for 30 minutes. The CAR contained an anti-CD19 scFv derived from a murine antibody (variable region derived from FMC63, an immunoglobulin-derived spacer, a transmembrane domain derived from CD28, a costimulatory region derived from 4-1BB, and a CD3-zeta intracellular signaling domain). The expression construct in the viral vector further contained sequences encoding a truncated receptor, which served as a surrogate marker for CAR expression, which was separated from the CAR sequence by a T2A ribosome skip sequence.

The spinoculated cells were washed and resuspended in media, and cultured at 37 degrees Celsius, until 96 hours after initiation of activation. Following culture, the transduced T cell composition (“input composition”) was loaded into the conical enclosure portion of a continuous counterflow centrifuge system (CTS Rotea™ Counterflow Centrifugation System) and subjected to a centrifugal force of 3,000 g and a flow rate of 10 mL/min for 30 seconds to establish a fluidized cell bed. The transduced cells were then subjected to a centrifugal force of 2,500 g and a flow rate of either 40 mL/min (62.5 G/FR) or 75 mL/min (33.3 G/FR). Under these conditions, the media was then exchanged into PlasmaLyte pH 7.4 with 1.25% human serum albumin as a “washing” step, allowing elutriation of non-viable cells for collection and analysis of the waste fraction.

As a control, transduced T cell compositions derived from the same donors and generated by the same method were subjected to an alternative method of automated, dead-end centrifugation and buffer exchange (the “alternative method”).

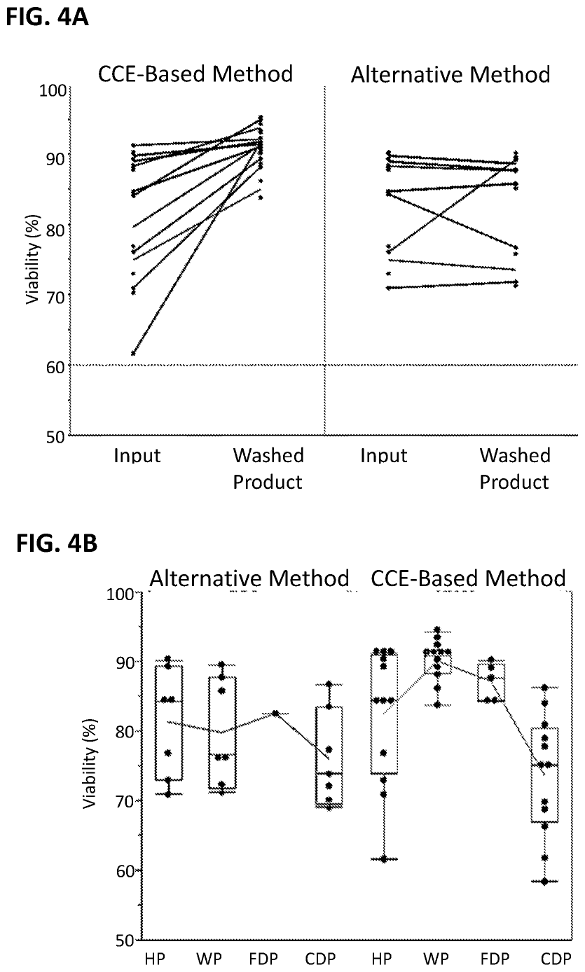

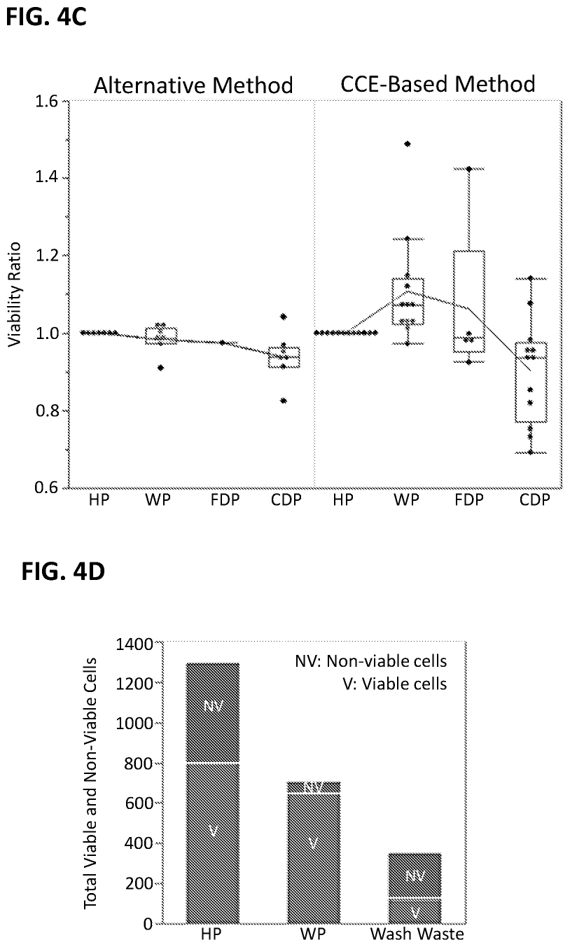

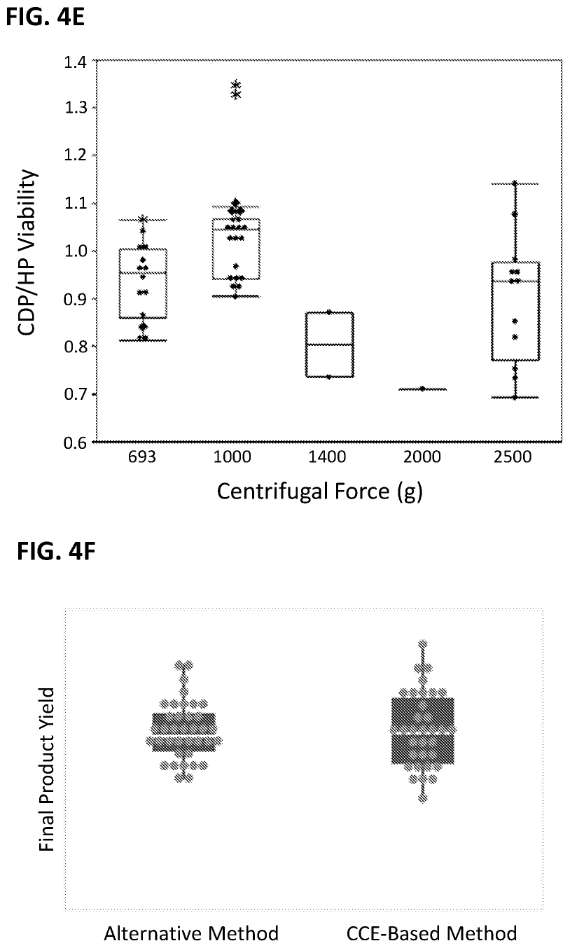

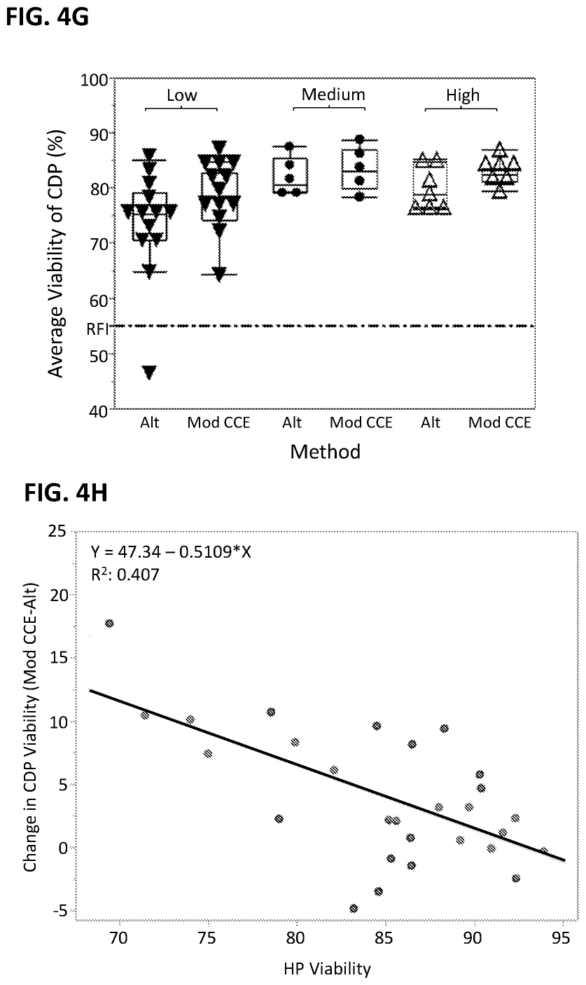

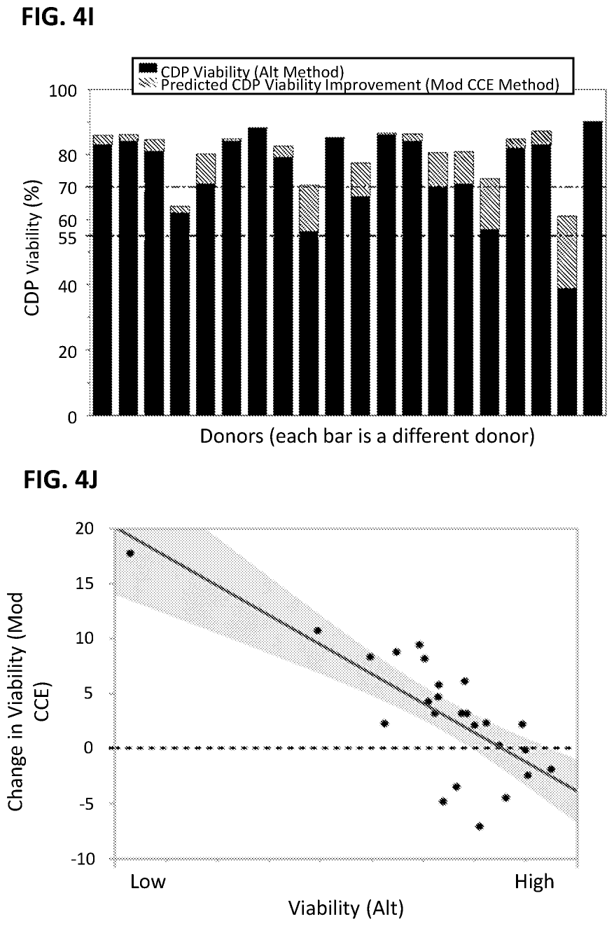

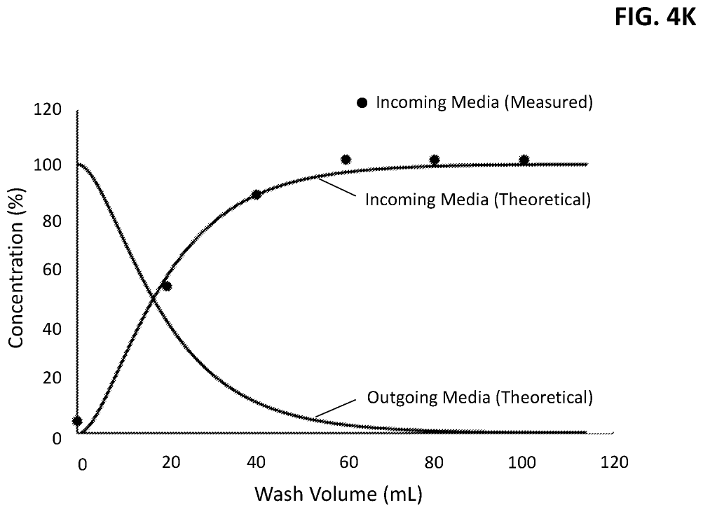

Cell viability of the input composition, washed product fraction, and waste fraction generated by the continuous counterflow-based method or the alternative method was assessed as previously described. As shown in FIG. 4A (each line represents a donor), continuous counterflow centrifugation of the input composition yielded consistently improved cell viability in washed product fractions, which was not observed in input compositions subjected to the alternative method of centrifugation and buffer exchange. The observations were similar for both G/FR ratios tested.

### Example 3 Maintenance of Improved Viability in Cryopreserved Product

Washed product (WP) fractions from Example 2 were subsequently re-suspended in 25% buffer and 75% CryoStor® CS-10 media to produce a formulated drug product (FDP). The viability of the FDP was assessed substantially as described. The improved cell viability observed in the washed product fraction was found to be maintained in the FDP. The FDP was then frozen in a controlled rate freezer to produce a cryopreserved drug product (CDP). The CDP was stored in liquid nitrogen for at least 3 days prior to removal for thaw at room temperature. The viability of the thawed CDP was observed to be decreased as compared to the viability of the pre-wash product fraction (“harvested product”; HP), the WP fraction, and the FDP (FIG. 4B).

To determine whether the loss of viability between the FDP and the thawed CDP was donor-dependent, the cell viability of the WP fraction, the FDP, and the thawed CDP for each donor was normalized to the initial viability of cells in the same donor's pre-wash product fraction (FIG. 4C). No donor-specific effects were observed. To further confirm that viable cells are enriched by the CCE-based centrifugation protocol in donor pre-wash product fractions having low viability, the number and location of cells during each step of the process were tracked in the donor pre-wash product fraction having the lowest viability. The pre-wash product (HP) fraction exhibited 61% cell viability, while the washed product (WP) fraction exhibited 92% viability (FIG. 4D), indicating that viable and non-viable cells are separated and distributed into different fractions during the CCE-based centrifugation, regardless of pre-wash product fraction viability.

The CCE-based centrifugation protocol was modified to reduce the loss in viability enrichment observed in thawed CDP (the “modified CCE-based method”). In particular, the centrifugal force and the flow rate were adjusted downward to reduce centrifugal stress on cells. As shown in FIG. 4E (input fractions with low viability (<70%) are designated by diamonds and asterisks), reducing the centrifugal force from 2500 g to 1000 g and implementing a flow rate of 28.5 mL/min (35 G/FR) preserved the viability enrichment effect in thawed CDP. The modified CCE-based method was also found to achieve comparable yields of cell numbers as compared to the alternative method (FIG. 4F). Without wishing to be bound by theory, these observations are consistent with a finding that a lower centrifugal force and flow rate can reduce the loss of cell viability observed in thawed CDP.

In a related experiment, low (230-400×10⁶ cells), medium (400-750×10⁶ cells), and high (>750×10⁶ cells) loads of cells from donor samples were subjected to either the modified CCE-based method (“Mod CCE”) or the alternative method (“Alt”) described in Example 2. Donor samples were matched between the respective modified CCE-based and alternative methods. The cell load was not observed to affect the viability of CDPs produced from the modified CCE-based method. In addition, the CDPs produced by the modified CCE-based method exhibited similar or higher viability compared to the alternative method (FIG. 4G), regardless of cell load.

Modeling was performed to predict the improvement in cell viability of CDPs produced from either the modified CCE-based method or the alternative method among donor samples exhibiting a range of viability in pre-wash product (HP) fractions. As shown in FIGS. 4H, 4I, and 4J the modeling predicted that the modified CCE-based method would yield increased viability enrichment compared to the alternative method, particularly among donors with pre-wash product fractions (“HP”) exhibiting lower viability. Thus, viability enrichment can be achieved while maintaining comparable final product yield. The ability of the CCE-based method to remove potential impurities (e.g., proteins, DNA, cell debris, and reagents used during manufacture) was also assessed by modeling of incoming and outgoing media, and by actual measurement of incoming media. In particular, FIG. 4K shows the predicted concentrations of an impurity in incoming and outgoing media, as well as the measured concentration of the impurity in incoming media.

Thus, the modeling indicates that the modified CCE-based method may increase the number of donors for whom product manufacturing is successful.

### Example 4 Enrichment of Transduced Viable Cells by Continuous Counterflow Centrifugation Following an Expanded Culture Protocol

A primary human T cell composition was generated and transduced as described in Example 2, except that the cells were subjected to an expanded protocol in which the T cells were cultured for a total of 15 days total following initiation of activation. By incubating the T cell composition for 15 days after initiation of activation, the cells may become less activated, thereby changing their size. Thus, experiments were undertaken to understand how the G/FR ratio affects the enrichment of viable cells subjected to the expanded protocol.

Following the expanded culture protocol, the transduced T cell input composition, exhibiting 50% cell viability, was loaded into the conical enclosure portion of a continuous counterflow centrifuge system (CTS Rotea™ Counterflow Centrifugation System) and subjected to a centrifugal force of 3,000 g and a flow rate of 10 mL/min for 30 seconds to establish a fluidized cell bed. The centrifugal force was then decreased to 2,500 g and the flow rate was increased to 40 mL/min, yielding a G/FR ratio of 62.5. As in Example 2, the media was exchanged into PlasmaLyte pH 7.4 with 1.25% human serum albumin as a “washing” step, allowing elutriation of non-viable cells for collection and analysis of the waste fraction.

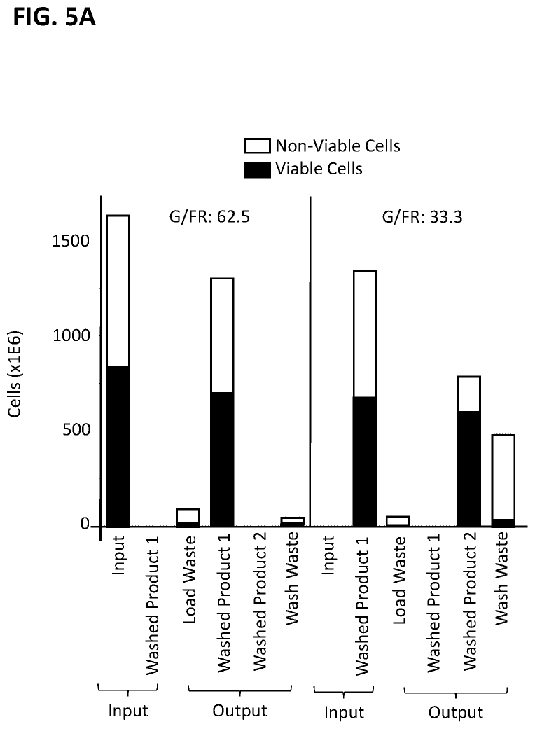

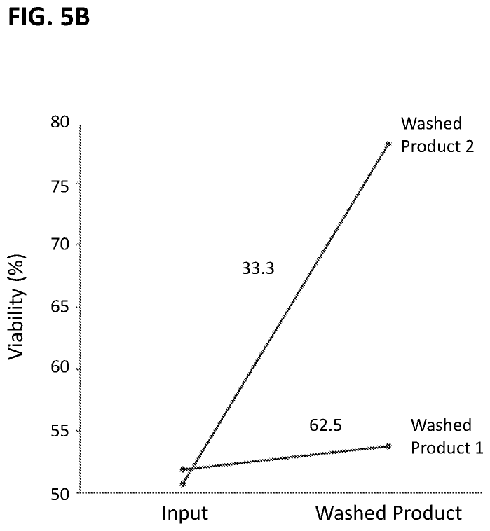

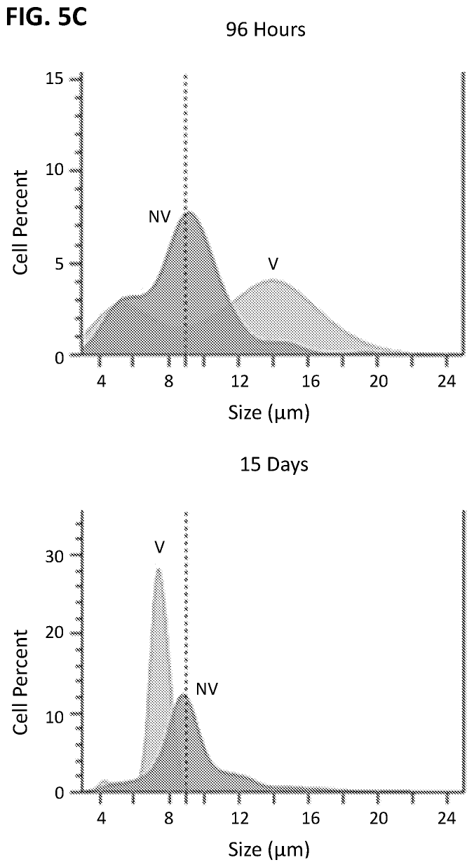

The number of viable and non-viable cells was assessed as previously described. As shown in FIG. 5A (left panel), when the centrifugal force and flow rate were held constant throughout the centrifugation process at 62.5 G/FR, the percentage of viable cells in the product fraction (Washed Product 1) was largely unchanged compared to the percentage of viable cells in the input composition (Input). In addition, there were minimal non-viable cells in the washed waste fraction (Wash Waste).

To elutriate additional non-viable cells, a second washing step was carried out on the cells collected during the first wash (Washed Product 1). During the second washing step, the centrifugal force was held at 2,500 g and flow rate was maintained at 75 mL/min, yielding a 33.3 G/FR ratio. Following the second wash at a G/FR ratio of 33.3, a significant number of non-viable cells were found in the washed waste fraction (Wash Waste) (FIG. 5A; right panel), and the percentage of viable cells in the product fraction (Washed Product 2) was significantly improved compared to that of the cells in the input composition (FIG. 5B). In combination with the results described in Example 3, a lower G/FR ratio (e.g., 33.3 or 35) is observed to improve enrichment for viable cells.

Without wishing to be bound by theory, changes in cell size after the expanded protocol may contribute to the differences in centrifugal force, flow rate, and ratios thereof that can be used for viability enrichment of expanded cells. The size of cells following the expanded protocol was determined based on cells of Washed Product 1, as shown in FIG. 5C (bottom panel). Viable (V) and non-viable (NV) cells of Washed Product 1 both had mean size less than 9 μm. For comparison, the size of cells after three days of activation in a separate experiment is shown in FIG. 5C (top panel). The cells activated for three days were on average larger than cells of Washed Product 1, which were collected 15 days after initiation of activation. This finding is consistent with cells increasing in size during initial activation, then becoming less activated and decreasing in size during expansion in culture. In addition, while non-viable cells (NV) of the cells activated for three days had a mean size of about 9 μm, a large proportion of viable cells (V) of the cells activated for three days had a larger size (about 14 μm).

### Example 5 Efficiency of Viral Transduction by Continuous Counterflow Centrifugation

Primary human T cell compositions were generated from leukapheresis samples from human adult donors and subjected to the CCE-based method of centrifugation in the presence of a vector encoding the anti-CD19 CAR to transduce the T cells, as in Example 1. Various parameters of the continuous counterflow centrifugation method were independently varied as described in the following sections, and transduction efficiency was assessed.

### A. Effect of Centrifugal Force and Flow Rate on Transduction Efficiency

A T cell composition was transduced with a lentiviral vector encoding an anti-CD19 CAR using a CCE-based method, substantially as described in Example 1, except that the centrifugal force and flow rates were varied. Following establishment of a fluidized bed of cells and vector particles, the centrifugal force was adjusted to either 625×g or 1,500×g, and the flow rate was adjusted to either 10 mL/min or 24 mL/min, yielding G/FR ratios of either 62.5 or 150 (see Table E1).

TABLE E1
Tested Force vs. Flow Rate

| Centrifuge (G) | 625 | 1500 | 1500 |
|---|---|---|---|
| Flow Rate (mL/min) | 10 | 24 | 10 |
| G/FR Ratio | 62.5 | 62.5 | 150 |

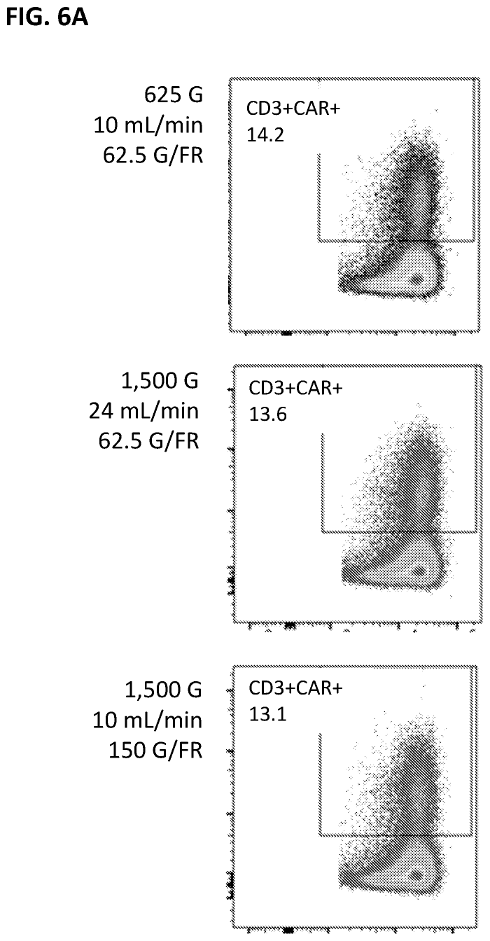

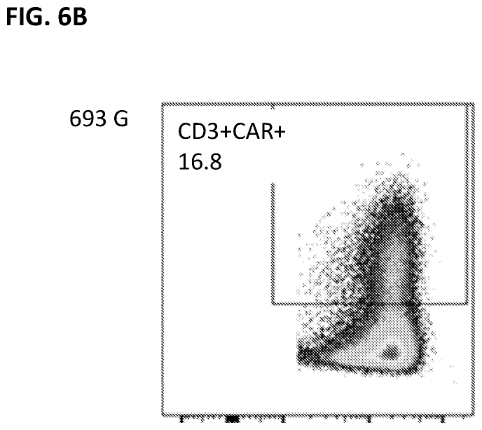

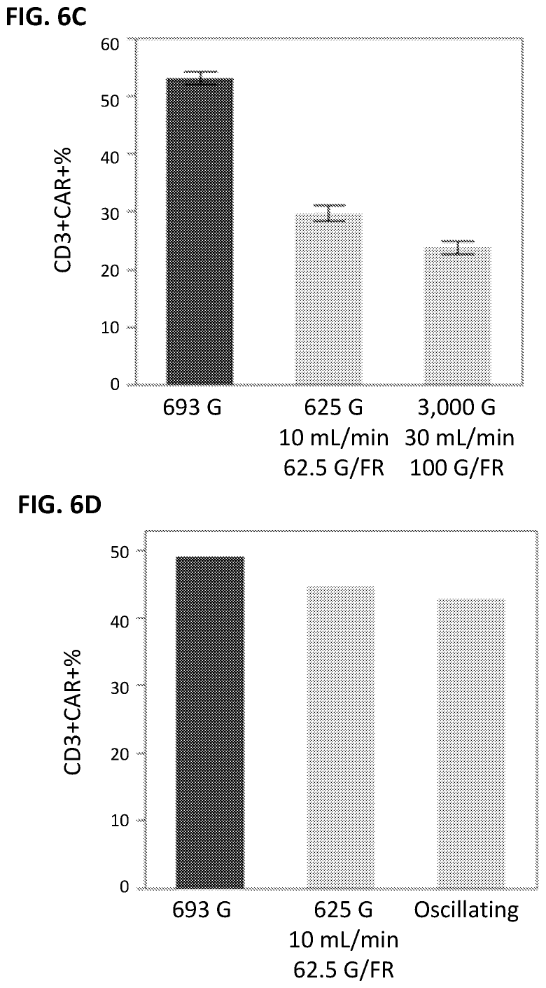

Transduction efficiency was calculated by determining the percentage of CD3+ T cells with surface expression of the CAR. As shown in FIG. 6A, T cell compositions were transduced with the vector encoding the CAR at the centrifugal force and flow rates set forth in Table E1. The different parameters tested resulted in similar percentages of CD3+CAR+ T cells.

As a control, the continuous counterflow centrifuge system-based method was compared to the scaled-down spinoculation method of transduction described in Subsection B of Example 1. Post-centrifugation vector integration was allowed to proceed as described in Example 1, and transduction efficiency was assessed. The results of the scaled-down spinoculation-based transduction are shown in FIG. 6B. The transduction efficiency of the two methods was observed to be comparable, such that the CCE-based method results in comparable transduction efficiency, but higher cell viability.

Further centrifugal force and flow rates were tested for recirculation, specifically 300G centrifugal force-to-10 mL/min flow rate (300G/10FR) and 3000G/30FR conditions. Recirculation at 300G/10FR resulted in comparable transduction efficiency to those achieved with the tested parameters in Table E1. As shown in FIG. 6C, recirculation at 3000G/30FR resulted in decreased transduction efficiency compared to those achieved with recirculation at 625G/10FR and the scaled-down spinoculation control method.

Another recirculation procedure was tested in which a 1500G/10FR condition was applied for one minute, followed by a 300G/10FR condition for one minute. This procedure was repeated over the 30-minute incubation, thereby oscillating the size of the fluidized bed in the conical enclosure throughout incubation. As shown in FIG. 6D, this oscillating recirculation procedure resulted in comparable transduction efficiency to that achieved with recirculation at 625G/10FR.

### B. Effect of Vector Titer on Transduction Efficiency

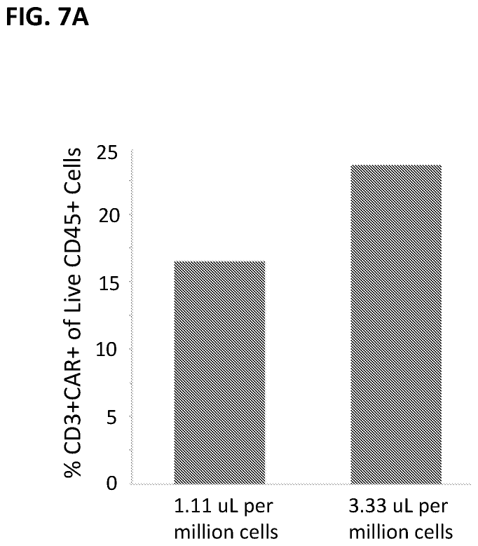

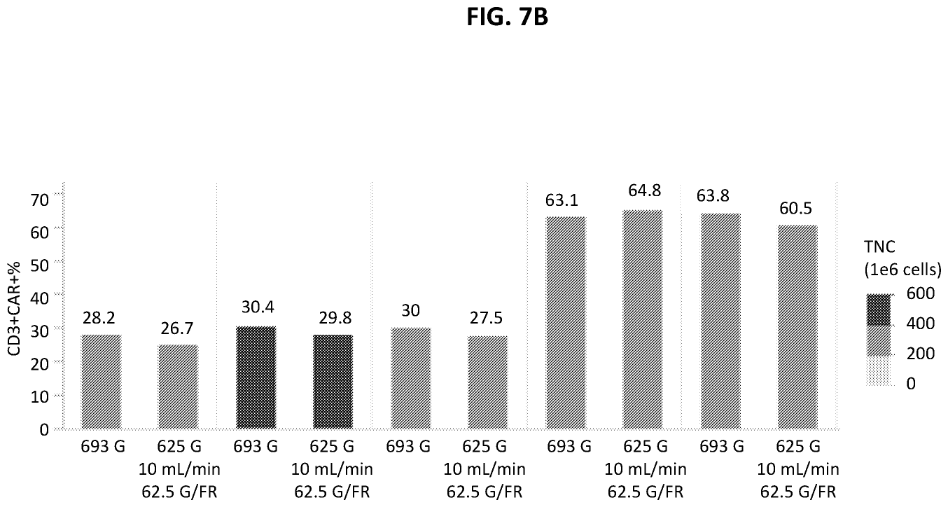

T cell compositions were transduced by the CCE-based method, substantially as described in the preceding Section, with either 1.11 or 3.33 μL of vector per million cells. As shown in FIG. 7A, the percent of CD3+CAR+ cells was increased when 3.33 μL of vector per million cells was used.

Further experiments were performed in which 6 μL of vector from a separate vector lot per million cells was used for transduction under 625G/10FR conditions for recirculation. As shown in FIG. 7B for transduced T cells from five donors, this vector titer resulted in transduction efficiency comparable to the at-scale spinoculation control method at 693G, indicating that comparable efficiency to the at-scale spinoculation control method could be achieved at optimal titers.

### C. Effect of Incubation Volume on Transduction Efficiency

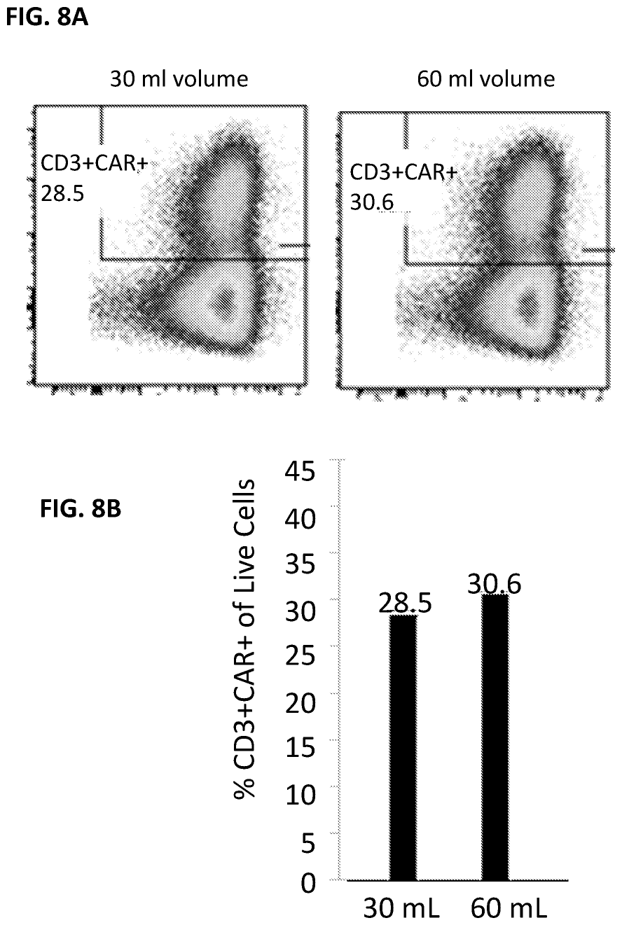

T cells were transduced in a continuous counterflow centrifuge system (CTS Rotea™ Counterflow Centrifugation System), substantially as described in the preceding Sections, except that the transduction volume was varied while the vector titer was held constant. T cells were centrifuged with 1.11 μL of vector per million cells in a total volume of either 30 mL or 60 mL (see Table E2). As shown in FIG. 8A and quantified in FIG. 8B, the results indicated that varying the transduction volume did not have a significant effect on the transduction efficiency of the CCE-based method. Similar results were achieved for transduced T cells produced from additional donors for which 25 mL, 35 mL, and 65 mL transduction volumes were tested.

TABLE E2
Effect of Incubation Volume on Transduction Efficiency

| | Vector Titer | Desired | Vector | Transduction | Relative | Transduction |
|---|---|---|---|---|---|---|
| | (μL/1 × 10⁶ | Transduction | Volume | Volume | Vector | VCC |
| | cells) | TNC (×1E6) | (μL) | (mL) | Concentration | (×1E6/mL) |
| CCE | 1.11 | 240.00 | 266.40 | 60.00 | 4.44 | 4.00 |
| CCE | 1.11 | 240.00 | 266.40 | 30.00 | 8.88 | 8.00 |

### D. Effect of T Cell Number on Transduction Efficiency

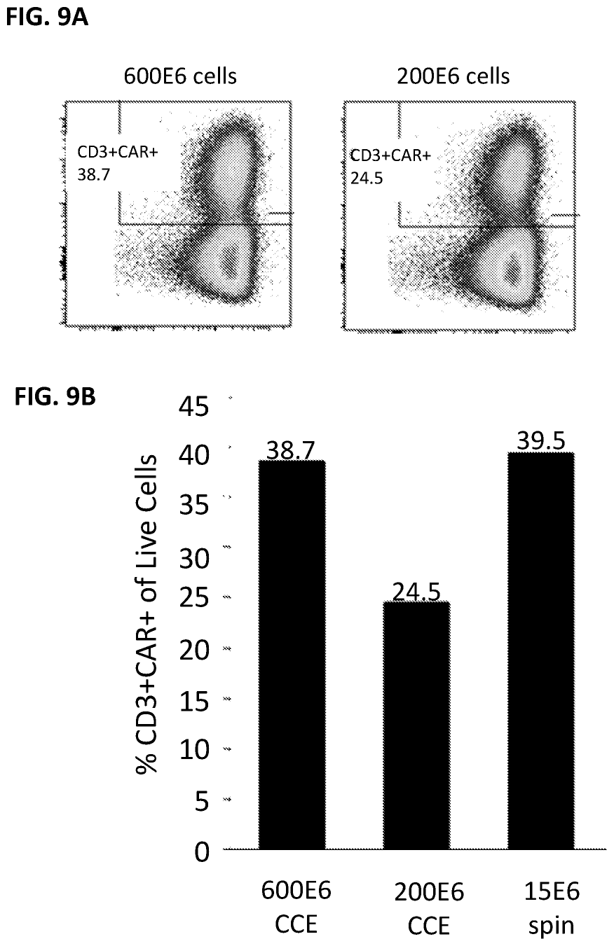

In another experiment, T cells were transduced in a continuous counterflow centrifuge system (CTS Rotea™ Counterflow Centrifugation System), substantially as described in in the preceding Sections, except that the number of T cells was varied while the transduction volume and vector titer were held constant. As shown in Table E3, either 200 or 600 million T cells were centrifuged with 1.11 μL of vector per million cells in a total volume of 35 mL. As a control, 15 million T cells were subjected to the scaled-down spinoculation method previously described. The composition containing 600 million T cells yielded a larger and denser fluidized cell bed than the composition containing 200 million T cells (data not shown). Transduction efficiency was determined as described in preceding Sections. The results, as shown in FIG. 9A and quantified in FIG. 9B, demonstrated that, between the CCE conditions, a higher number of T cells in the input composition resulted in a higher transduction rate.

TABLE E3
Effect of T Cell Number on Transduction Efficiency

| | Vector Titer | Desired | Vector | Transduction | Relative |
|---|---|---|---|---|---|
| | (μL/1 × 10⁶ | Transduction | Volume | Volume | Vector |
| | cells) | TNC (×1E6) | (μL) | (mL) | Concentration |
| Spinoculation | 1.11 | 15 | 16.65 | 0.76 | 21.91 |
| CCE | 1.11 | 200 | 222 | 35 | 6.34 |
| CCE | 1.11 | 600 | 666 | 35 | 19.03 |

### E. Effect of Incubation Time on Transduction Efficiency

200×10⁶ T cells were transduced in a continuous counterflow centrifuge system (CTS Rotea™ Counterflow Centrifugation System), substantially as described in in the preceding Sections, except that incubation time was varied by allowing the output fraction to recirculate through the conical enclosure of the continuous counterflow centrifuge system for either 30 minutes or 90 minutes. Supernatant samples were periodically collected from the centrifuge chamber to monitor consumption of the lentiviral vector over time. Jurkat cells were transduced with the samples, and the quantity of lentiviral vector in each supernatant sample was assessed based on the transduction efficiency. The transduction efficiency of T cells transduced by the continuous counterflow centrifuge system was also assessed.

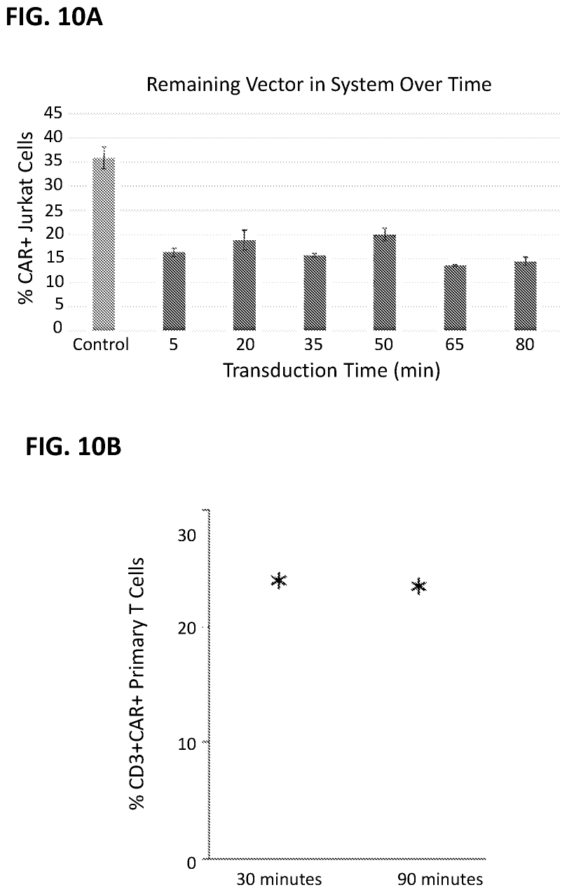

As shown in FIG. 10A, the percent of Jurkat cells transduced by the lentiviral vector supernatant sample obtained after 5 minutes of incubation was reduced by approximately 20% compared to Jurkat cells transduced by starting lentiviral vector material. As shown in FIG. 10B, no significant difference in transduction efficiency was observed between the T cells incubated with the lentiviral vector particles for 30 minutes and those incubated with the particles for 90 minutes. Without wishing to be bound by theory, the results of this experiment indicate that the majority of the lentiviral vector is consumed within the first five minutes of incubation, such that longer incubation times may not increase transduction efficiency.

### F. Effect of Centrifuge System Volume on Vector Concentration

T cells were transduced using the CCE-based method described in Example 1 under 625G/10FR conditions for recirculation, except that the total volume of the continuous counterflow centrifuge system was increased by the addition of an incubation bag to the fluid path. The total system volume without the incubation bag was 25 mL. The total system volume was increased to 39 mL or 72 mL by the addition of an incubation bag. The volume of the conical enclosure portion was 15 mL.

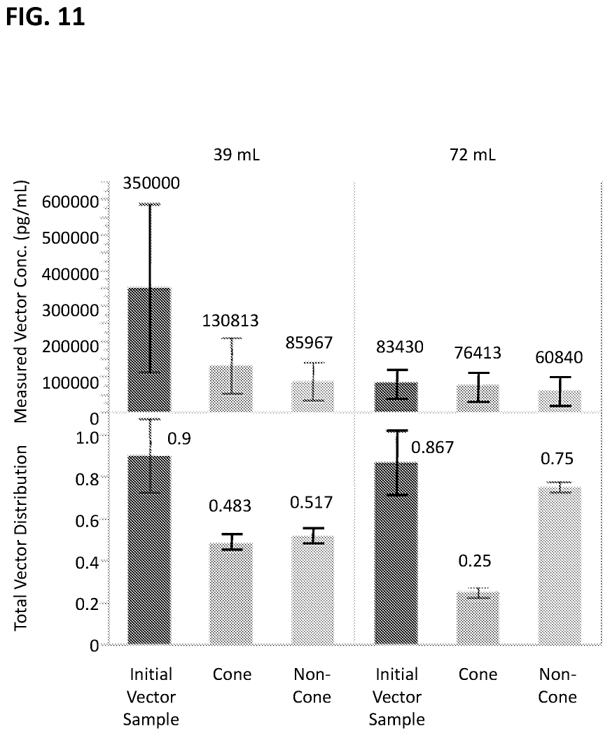

Increasing the total system volume from 39 mL to 72 mL did not affect transduction efficiency. Further analysis revealed that in the 39-mL system, the viral vector was preferentially concentrated in the conical enclosure portion, where a centrifugal field was present and despite the conical enclosure portion accounting for only 15 mL of the total system volume (FIG. 11, left panels). In the 72-mL system, less preferential concentration of the viral vector was observed (FIG. 11, right panels).

### Example 6 Debeading of a Bead-Cell Composition by Centrifugal Elutriation

A primary human T cell composition was generated as described in Example 1. The T cells were incubated with approximately 200×10⁶ paramagnetic polystyrene beads coated with anti-CD3 and anti-CD28 antibodies for 24 hours at 37 degrees Celsius to stimulate the T cells. Following stimulation, the cell and bead composition was diluted to 7.5×10⁵ cells/mL and further incubated at 37 degrees Celsius for 72 hours. After the further incubation, the cell composition was determined to contain 1.60×10⁶ cells/mL with 66% viability, as analyzed by an automated cell counter. The cell-bead composition was then placed into the conical fluid enclosure portion of a continuous counterflow centrifuge system (CTS Rotea™ Counterflow Centrifugation System) as an “input” composition, and subjected to a continuous counterflow elutriation (“CCE”)-based method of centrifugation. The input composition was initially subjected to a centrifugal force of 1,000 g and a flow rate of 30 mL/min to establish a fluidized bed of the cell-bead composition in the conical fluid enclosure.

The fluidized bed of the cell-bead composition was subsequently subjected to a flow rate of 50 mL/min. The centrifugal force was decreased stepwise from 1,000 g to 200 g in 100 g increments. Samples were taken at intervals for analysis of cell number and viability. Based on the data collected, a flow rate of 50 mL/min and a centrifugal force of 600 g were chosen for subsequent experiments.

In a subsequent experiment, a T cell input composition was generated to contain approximately 200×10⁶ beads and 2.08×10⁶ cells/mL with 69.5% viability. After being initially subjected to a centrifugal force of 1,000 g and a flow rate of 30 mL/min to establish a fluidized bed of the cell-bead composition, the input composition was subjected to a centrifugal force of 600 g and a flow rate of 50 mL/min, resulting in an “output” fraction of T cells that was recirculated through the continuous counterflow centrifuge system in a fluid path, until it was redirected to a final collection chamber as a “product” fraction.

The number of beads in the input composition and the product and waste fractions were determined using a fluorescent microscopy-based Cytation™ 5 cell imaging system. The final product fraction contained 220 beads/mL, representing approximately 0.01% of the bead particles present in the input composition. The collected waste fraction contained 40,930 beads/mL. The number of cells and bead particles in the output fraction are shown in Table E4.

TABLE E4
Cell-Bead Composition Debeading

| | | | Bead Yield |
|---|---|---|---|
| | Total Beads | | (based on 200 × 10⁶ beads |
| Fraction | Calculated | Beads/mL | in input composition) |
| Input | 3.66 × 10⁶ | 31,618 | N/A |
| Product₁ | 2.87 × 10⁴ | 220 | 0.01% |
| Product₂ | 3.64 × 10⁴ | 343 | 0.02% |
| Waste | 2.61 × 10⁶ | 40,930 | 1.31% |

Product₁ is the first elutriation fraction after passing the entire input composition through the chamber.
Product₂ is the second elutriation fraction after rinsing the chamber with buffer.

### Example 7 Additional Methods for Transduction in a Continuous Counterflow Centrifuge System

Additional methods for transducing cells in a continuous counterflow centrifuge system (CTS Rotea™ Counterflow Centrifugation System) were tested for their effects on transduction efficiency. Other than the modifications described below, the additional methods were otherwise as described in Example 1.

In one additional method that was tested, T cells and lentiviral vector particles were incubated for one minute after the fluidized bed of cells and vector particles was established, after which the transduced product faction was then immediately collected without recirculation. This method resulted in reduced transduction efficiency relative to that achieved with the 625G/10FR CCE method described in Example 1.

In another additional method that was tested, T cells and lentiviral vector particles were incubated for 30 minutes under 625G/10FR conditions after the fluidized bed of cells and vector particles was established. However, during incubation, flow in the fluid path was directed radially outward through the tip of the conical enclosure portion, rather than in counter flow radially inward through the center of the conical enclosure portion. This method resulted in comparable transduction efficiency to that achieved with the 625G/10FR CCE method described in Example 1.

In another additional method that was tested, the fluidized bed of cells and vector particles was established at 2500G/40FR and incubated for one minute at 625G/10FR, after which flow was reversed, still at 625G/10FR, to force cells and vector particles from the fluidized bed through the narrow cannula running along the length of the conical enclosure portion from the tip to the center of the conical enclosure portion. Cells and viral particles were collected from the cannula over a one-minute period, after which the fluidized bed of cells and vector particles was re-established with the uncollected cells and vector particles. This procedure was repeated over a 30-minute incubation. This method resulted in comparable transduction efficiency to that achieved with the 625G/10FR CCE method described in Example 1.

Together, these results establish that other methods for transduction in a continuous counterflow centrifuge system can be performed, including non-counterflow methods or methods involving periodic harvest of cells from the continuous counterflow centrifuge system.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO | Sequence | Description |
| 1 | ESKYGPPCPPCP | spacer |
| | | (IgG4hinge) |
| | | (aa) |
| 2 | gaatctaagtacggaccgccctgccccccttgccct | spacer |
| | | (IgG4hinge) |
| | | (nt) |
| 3 | ESKYGPPCPPCPGQPREPQVYTLPPSQEEMTKNQVSLTCLVKGFY | Hinge-CH3 |
| | PSDIAVEWESNGQPENNYKTTPPVLDSDGSFFLYSRLTVDKSRW | spacer |
| | QEGNVFSCSVMHEALHNHYTQKSLSLSLGK | |
| 4 | ESKYGPPCPPCPAPEFLGGPSVFLFPPKPKDTLMISRTPEVTCVVV | Hinge-CH2- |
| | DVSQEDPEVQFNWYVDGVEVHNAKTKPREEQFNSTYRVVSVLT | CH3 spacer |
| | VLHQDWLNGKEYKCKVSNKGLPSSIEKTISKAKGQPREPQVYTL | |
| | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| 5 | RWPESPKAQASSVPTAQPQAEGSLAKATTAPATTRNTGRGGEEK | IgD-hinge-Fc |
| | KKEKEKEEQEERETKTPECPSHTQPLGVYLLTPAVQDLWLRDKA | |
| | TFTCFVVGSDLKDAHLTWEVAGKVPTGGVEEGLLERHSNGSQS | |
| | QHSRLTLPRSLWNAGTSVTCTLNHPSLPPQRLMALREPAAQAPV | |
| | KLSLNLLASSDPPEAASWLLCEVSGFSPPNILLMWLEDQREVNTS | |
| | GFAPARPPPQPGSTTFWAWSVLRVPAPPSPQPATYTCVVSHEDSR | |
| | TLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIK | tEGFR |
| | HFKNCTSISGDLHILPVAFRGDSFTHTPPLDPQELDILKTVKEITG | |
| | FLLIQAWPENRTDLHAFENLEIIRGRTKQHGQFSLAVVSLNITSLG | |
| | LRSLKEISDGDVIISGNKNLCYANTINWKKLFGTSGQKTKIISNRG | |
| | ENSCKATGQVCHALCSPEGCWGPEPRDCVSCRNVSRGRECVDKCN | |
| | LLEGEPREFVENSECIQCHPECLPQAMNITCTGRGPDNCIQCAHYI | |
| | DGPHCVKTCPAGVMGENNTLVWKYADAGHVCHLCHPNCTYGC | |
| | TGPGLEGCPTNGPKIPSIATGMVGALLLLLVVALGIGLFM | |
| 8 | FWVLVVVGGVLACYSLLVTVAFIIFWV | CD28 (amino |
| | | acids 153-179 |
| | | of Accession |
| | | No. P10747) |
| 9 | IEVMYPPPYLDNEKSNGTIIHVKGKHLCPSPLFPGPSKPFWVLVV | CD28 (amino |
| | VGGVLACYSLLVTVAFIIFWV | acids 114-179 |
| | | of Accession |
| | | No. P10747) |
| 10 | RSKRSRLLHSDYMNMTPRRPGPTRKHYQPYAPPRDFAAYRS | CD28 (amino |
| | | acids 180-220 |
| | | of P10747) |
| 11 | RSKRSRGGHSDYMNMTPRRPGPTRKHYQPYAPPRDFAAYRS | CD28 (LL to |
| | | GG) |
| 12 | KRGRKKLLYIFKQPFMRPVQTTQEEDGCSCRFPEEEEGGCEL | 4-1BB (amino |
| | | acids 214-255 |
| | | of Q07011.1) |
| 13 | RVKFSRSADAPAYQQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 14 | RVKFSRSAEPPAYQQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 15 | RVKFSRSADAPAYKQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 16 | RKVCNGIGIGEFKDSLSINATNIKHFKNCTSISGDLHILPVAFRGD | tEGFR |
| | SFTHTPPLDPQELDILKTVKEITGFLLIQAWPENRTDLHAFENLEI | |
| | IRGRTKQHGQFSLAVVSLNITSLGLRSLKEISDGDVIISGNKNLCY | |
| | ANTINWKKLFGTSGQKTKIISNRGENSCKATGQVCHALCSPEGCW | |
| | GPEPRDCVSCRNVSRGRECVDKCNLLEGEPREFVENSECIQCHPE | |
| | CLPQAMNITCTGRGPDNCIQCAHYIDGPHCVKTCPAGVMGENNT | |
| | LVWKYADAGHVCHLCHPNCTYGCTGPGLEGCPTNGPKIPSIATG | |
| | MVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P- wherein P is proline, G is | Linker |
| | glycine and S is serine | |
| 23 | GSADDAKKDAAKKDGKS | Linker |
| 24 | atgcttctcctggtgacaagccttctgctctgtgagttaccacacc | GMCSFR |
| | cagcattcctcctgatccca | alpha chain |
| | | signal |
| | | sequence |
| 25 | MLLLVTSLLLCELPHPAFLLIP | GMCSFR |
| | | alpha chain |
| | | signal |
| | | sequence |
| 26 | MALPVTALLLPLALLLHA | CD8 alpha |
| | | signal peptide |

1. A method for producing a composition of genetically engineered T cells, the method comprising:
(a) applying a first centrifugal force and a first flow rate to a cell composition comprising T cells in a conical fluid enclosure of a centrifuge system to produce a fluidized bed of cells;
(b) loading a viral vector particle into the conical fluid enclosure, thereby generating an input composition comprising the cell composition and the viral vector particle; and
(c) applying a second centrifugal force and a second flow rate to the input composition, wherein the second centrifugal force and second flow rate recirculate the viral vector particle in a fluid path of the centrifuge system, thereby generating genetically engineered T cells.

2. The method of claim 1, wherein the loading of the viral vector particle is carried out during at least a portion of the applying in (a) and/or is carried out during at least a portion of the applying in (c).

3. A method for producing a composition of genetically engineered T cells, the method comprising:
(a) applying a first centrifugal force and a first flow rate to an input composition comprising (i) a viral vector particle and (ii) a cell composition comprising T cells in a conical fluid enclosure of a centrifuge system to produce a fluidized bed of cells; and
(b) applying a second centrifugal force and a second flow rate to the input composition in the conical fluid enclosure, wherein the second centrifugal force and second flow rate recirculate the viral vector particle in a fluid path of the centrifuge system, thereby generating genetically engineered T cells.

4. The method of claim 3, further comprising loading the cell composition and the viral vector particle into the conical fluid enclosure, thereby generating the input composition, wherein the loading of the cell composition is before, during, and/or after the loading of the viral vector particle.

5. The method of claim 4, wherein the loading of the cell composition and/or the loading of the viral vector particle is performed prior to and/or during the applying in (a).

6. The method of any one of claims 1-5, wherein the centrifuge system is a continuous counterflow centrifuge system.

7. The method of any one of claims 1-6, further comprising applying a third centrifugal force and a third flow rate to the genetically engineered T cells in the conical fluid enclosure of the centrifuge system to produce an output composition comprising the genetically engineered T cells.

8. The method of claim 7, wherein the percentage of viable T cells in the output composition is greater than the percentage of viable T cells in the input composition, optionally at least about 5% greater, at least about 10% greater, at least about 15% greater, at least about 20% greater, or at least about 25% greater.

9. The method of claim 7 or claim 8, wherein at least or at least about 5%, at least or at least about 10%, at least or at least about 15%, at least or at least about 20%, at least or at least about 25%, or at least or at least about 30% of the T cells in the output composition are transduced with the viral vector particle.

10. The method of any one of claims 1-9, wherein (i) the first centrifugal force is between about 2,000 G and about 4,000 G; and (ii) the first flow rate is between about 5 mL/min and about 15 mL/min, optionally wherein the first centrifugal force and the first flow rate are applied to the cell composition or the input composition for about 15 seconds, about 30 seconds, about 45 seconds, or about 60 seconds.

11. The method of any one of claims 1-10, wherein (i) the second centrifugal force is between about 500 G and about 1,500 G; and (ii) the second flow rate is between about 25 mL/min and about 30 mL/min.

12. The method of any one of claims 1-11, wherein the ratio of the first centrifugal force (in G) to the first flow rate (in mL/min) is between about 200 and about 400.

13. The method of any one of claims 1-12, wherein the ratio of the first centrifugal force (in G) to the first flow rate (in mL/min) is about 300.

14. The method of any one of claims 1-13, wherein the first centrifugal force is about 3,000 G, and the first flow rate is about 10 mL/min.

15. The method of any one of claims 1-14, wherein the ratio of the second centrifugal force (in G) to the second flow rate (in mL/min) is between about 20 and about 100, between about 25 and about 85, or between about 30 and about 65.

16. The method of any one of claims 1-15, wherein the ratio of the second centrifugal force (in G) to the second flow rate (in mL/min) is about 35.

17. The method of any one of claims 1-16, wherein the second centrifugal force is about 1,000 G, and the second flow rate is about 28.5 mL/min.

18. The method of any one of claims 1-10 and 12-16, wherein (i) the second centrifugal force is between about 500 G and about 1,500 G; and (ii) the second flow rate is between about 10 mL/min and about 100 mL/min.

19. The method of any one of claims 1-15 and 18, wherein the ratio of the second centrifugal force (in G) to the second flow rate (in mL/min) is about 62.5.

20. The method of any one of claims 1-10, 12-15, 18, and 19, wherein the second centrifugal force is about 625 G, and the second flow rate is about 10 mL/min.

21. The method of any one of claims 1-10, 12-16, and 19, wherein (i) the second centrifugal force is between about 100 G and about 2,000 G; and (ii) the second flow rate is between about 10 mL/min and about 100 mL/min.

22. The method of any one of claims 1-15, 18, and 21, wherein the ratio of the second centrifugal force (in G) to the second flow rate (in mL/min) is about 30.

23. The method of any one of claims 1-10, 12-15, 21, and 22, wherein the second centrifugal force is about 300 G, and the second flow rate is about 10 mL/min.

24. The method of any one of claims 1-23, wherein the second centrifugal force and the second flow rate are applied to the input composition for at least about 15 minutes, at least about 30 minutes, at least about 45 minutes, at least about 60 minutes, at least about 75 minutes, or at least about 90 minutes.

25. The method of any one of claims 7-24, wherein (i) the third centrifugal force is between about 2,000 G and about 3,000 G; and (ii) the third flow rate is between about 15 mL/min and about 25 mL/min.

26. The method of any one of claims 7-25, wherein the ratio of the third centrifugal force (in G) to the third flow rate (in mL/min) is between about 100 and about 150, optionally wherein the ratio of the third centrifugal force (in G) to the third flow rate (in mL/min) is about 125.

27. The method of any one of claims 7-26, wherein the third centrifugal force is about 2,500 G, and the third flow rate is about 20 mL/min.

28. The method of any one of claims 7-27, wherein prior to the applying the third centrifugal force and the third flow rate, the method comprises subjecting the genetically engineered T cells to one or more washing steps, optionally wherein the one or more washing steps comprise media exchange.

29. The method of any one of claims 1-28, wherein the method comprises incubating the T cells of the cell composition under stimulating conditions prior to the applying in (a) and/or the T cells of the cell compositions are incubated under stimulating conditions prior to the applying in (a).

30. The method of claim 29, wherein the stimulating conditions comprise the presence of a stimulatory reagent that is capable of activating one or more intracellular signaling domains of one or more components of a TCR complex and one or more intracellular signaling domains of one or more costimulatory molecules.

31. The method of claim 30, wherein the stimulatory reagent comprises (i) a primary agent that specifically binds to a member of a TCR complex, optionally that specifically binds to CD3; and (ii) a secondary agent that specifically binds to a T cell costimulatory molecule, optionally wherein the costimulatory molecule is selected from CD28, CD137 (4-1-BB), OX40, and ICOS.

32. The method of claim 31, wherein at least one of the primary and secondary agents comprises an antibody or an antigen-binding fragment thereof.

33. The method of claim 31 or claim 32, wherein the primary agent is an anti-CD3 antibody or an antigen-binding fragment thereof, and the secondary agent is an anti-CD28 antibody or an antigen-binding fragment thereof.

34. The method of any one of claims 31-33, wherein the primary agent and the secondary agent are each present on the surface of a solid support, optionally wherein the primary agent and the secondary agent are each present on the surface of a bead, further optionally a paramagnetic bead.

35. The method of any one of claims 31-33, wherein the primary agent and the secondary agent are reversibly bound on the surface of an oligomeric particle reagent comprising a plurality of streptavidin molecules or streptavidin mutein molecules.

36. The method of claim 35, wherein the streptavidin molecules or the streptavidin mutein molecules bind to or are capable of binding to biotin or a biotin analog.

37. The method of any one of claims 31-36, wherein the primary agent comprises an anti-CD3 Fab, and the secondary agent comprises an anti-CD28 Fab.

38. The method of any one of claims 29-37, wherein the stimulating conditions comprise the presence of one or more recombinant cytokines.

39. The method of any one of claims 29-38, wherein the stimulating conditions comprise the presence of one or more of recombinant IL-2, IL-7, and IL-15.

40. The method of any one of claims 7-39, wherein the method comprises collecting the output composition and/or the output composition is collected.

41. The method of claim 40, wherein the method comprises incubating the genetically engineered T cells of the collected output composition and/or the genetically engineered T cells of the collected output composition are incubated.

42. The method of claim 40 or claim 41, wherein the genetically engineered T cells of the collected output composition are incubated immediately following the collecting for at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days.

43. The method of any one of claims 40-42, wherein the percentage of viable T cells in the collected output composition about 1 day after, about 2 days after, about 3 days after, about 4 days after, about 5 days after, about 6 days after, about 7 days after, about 8 days after, about 9 days after, or about 10 days after collection is greater than the percentage of viable T cells in the input composition.

44. The method of any one of claims 40-43, wherein the percentage of viable T cells in the collected output composition about 1 day after collection is greater than the percentage of viable T cells in the input composition.

45. The method of any one of claims 40-44, wherein the percentage of viable T cells in the collected output composition about 5 days after collection is greater than the percentage of viable T cells in the input composition.

46. The method of any one of claims 40-45, wherein the method comprises cryopreserving the collected output composition and/or the collected output composition is cryopreserved, thereby generating a cryopreserved composition.

47. The method of claim 46, wherein the cryopreserved composition is thawed to produce a thawed composition, and the percentage of viable T cells in the thawed composition is greater than the percentage of viable T cells in the input composition, optionally at least about 5% greater, at least about 10% greater, at least about 15% greater, at least about 20% greater, at least about 25% greater, or at least about 30% greater.

48. The method of any one of claims 1-47, wherein the input composition comprises T cells having an average diameter of greater than or greater than about 6 microns, greater than or greater than about 6 microns, greater than or greater than about 7 microns, greater than or greater than about 8 microns, greater or greater than about 9 microns, greater or greater than about 10 microns, or greater or greater than about 11 microns.

49. The method of any one of claims 1-48, wherein the input composition comprises between about 1×10⁶ total T cells and about 2×10⁹ total T cells.

50. The method of any one of claims 1-49, wherein the input composition comprises at least about 1×10⁸ total T cells, at least about 2×10⁸ total T cells, at least about 3×10⁸ total T cells, at least about 4×10⁸ total T cells, at least about 5×10⁸ total T cells, at least about 6×10⁸ total T cells, at least about 7×10⁸ total T cells, at least about 8×10⁸ total T cells, at least about 7×10⁸ total T cells, at least about 8×10⁸ total T cells, at least about 9×10⁸ total T cells, at least about 1×10⁹ total T cells, at least about 1.25×10⁹ total T cells, at least about 1.50×10⁹ total T cells, or at least about 1.75×10⁹ total T cells.

51. The method of any one of claims 1-50, wherein the volume of the input composition is between about 5 ml and about 20,000 ml, between about 10 mL and about 2,000 mL, between about 15 mL and about 1,000 mL, between about 20 mL and about 500 mL, between about 25 mL and about 100 mL, or between about 30 mL and about 60 mL.

52. The method of any one of claims 1-51, wherein the volume of the input composition is between about 30 mL and about 60 mL.

53. The method of any one of claims 7-52, wherein the volume of the output composition is between about 2.5 mL and about 60 mL, between about 5 mL and about 40 mL, or between about 10 mL and about 20 mL.

54. The method of any one of claims 7-53, wherein the volume of the output composition is about 5 mL, about 10 mL, about 15 mL, about 20 mL, about 25 mL, about 30 mL, about 35 mL, about 40 mL, about 45 mL, about 50 mL, about 55 mL, or about 60 mL.

55. A method for enriching a cell composition for viable cells, the method comprising:
(a) applying a first centrifugal force and a first flow rate to a cell composition comprising T cells in a conical fluid enclosure of a centrifuge system to produce a fluidized bed of cells, wherein the cell composition comprises viable and non-viable T cells, and
(b) applying a second centrifugal force and a second flow rate to the cell composition, wherein the second centrifugal force and second flow rate recirculate cells of the cell composition in a fluid path of the centrifuge system, thereby elutriating out of the conical fluid enclosure a waste fraction of the cell composition that has a higher percentage of nonviable T cells than the percentage of nonviable T cells in the cell composition and producing within the conical fluid enclosure an enriched composition that has a higher percentage of viable T cells than the percentage of viable T cells in the cell composition.

56. The method of claim 55, wherein (i) the first centrifugal force is between about 1,000 G and about 4,000 G; and (ii) the first flow rate is between about 5 mL/min and about 15 mL/min.

57. The method of claim 55 or claim 56, wherein the ratio of the first centrifugal force (in G) to the first flow rate (in mL/min) is between about 200 and about 500.

58. The method of any one of claims 55-57, wherein the ratio of the first centrifugal force (in G) to the first flow rate (in mL/min) is between about 200 and about 400.

59. The method of any one of claims 55-58, wherein the first centrifugal force and the first flow rate are applied to the cell composition for at least 30 seconds.

60. The method of any one of claims 55-59, wherein (i) the second centrifugal force is between about 350 G and about 4,000 G; and (ii) the second flow rate is between about 5 mL/min and about 100 mL/min.

61. The method of any of claims 55-60, wherein the second centrifugal force is between about 350 G and 3,000 G.

62. The method of any of claims 55-61, wherein the second centrifugal force is between about 1,500 G and about 3,000 G.

63. The method of any of claims 55-61, wherein the second centrifugal force is between about 500 G and about 1,500 G.

64. The method of any of claims 55-63, wherein the second flow rate is between about 65 mL/min and about 100 mL/min.

65. The method of any one of claims 55-63, wherein the second flow rate is between about 10 mL/min and about 65 mL/min.

66. The method of any one of claims 55-63 and 65, wherein the second flow rate is between about 10 mL/min and about 35 mL/min.

67. The method of any one of claims 55-63, 65, and 66, wherein the second flow rate is between about 25 mL/min and about 30 mL/min.

68. The method of any one of claims 55-67, wherein the ratio of the second centrifugal force (in G) to the second flow rate (in mL/min) is between about 30 and about 70.

69. The method of any one of claims 55-68, wherein the ratio of the second centrifugal force (in G) to the second flow rate (in mL/min) is between about 30 and about 40.

70. The method of any one of claims 1-69, wherein the T cells have a mean diameter of about 9 μm to about 20 μm.

71. The method of any one of claims 55-68 and 70, wherein the T cells have a mean diameter of about 9 μm to about 20 μm, and the ratio of the second centrifugal force (in G) to the second flow rate (in mL/min) is between about 30 and about 70.

72. The method of any one of claims 1-69, wherein the T cells have a mean diameter of less than 9 μm.

73. The method of any one of claims 55-69 and 72, wherein the T cells have a mean diameter of less than 9 μm, and the ratio of the second centrifugal force (in G) to the second flow rate (in mL/min) is between about 30 and about 40.

74. A method of enriching a cell composition for viable cells, the method comprising:
(a) applying a first centrifugal force and a first flow rate to a cell composition comprising T cells in a conical fluid enclosure of a centrifuge system to produce a fluidized bed of cells, wherein the cell composition comprises viable and non-viable T cells, wherein (i) the first centrifugal force is between about 2,000 G and about 4,000 G; and (ii) the first flow rate is between about 5 mL/min and about 15 mL/min; and
(b) applying a second centrifugal force and a second flow rate to the cell composition, wherein the second centrifugal force and second flow rate recirculate cells of the cell composition in a fluid path of the centrifuge system, thereby generating an enriched composition having a higher percentage of viable T cells than the percentage of viable T cells in the cell composition, wherein (i) the second centrifugal force is between about 1,500 G and about 3,000 G; (ii) the second flow rate is between about 65 mL/min and about 100 mL/min; and (iii) the ratio of the second centrifugal force (in G) to the second flow rate (in mL/min) is between about 30 and about 40;
wherein the T cells have a mean diameter of less than 9 μm.

75. The method of any one of claims 1-69 and 72-74, wherein the T cells have a mean diameter of about 6 μm to about 9 μm.

76. The method any one of claims 55-75, wherein the method comprises collecting the elutriated waste fraction.

77. The method of claim 76, wherein the elutriated waste fraction is collected in a container that is in fluid communication with the wide end of the conical fluid enclosure.

78. A method for enriching a cell composition for viable cells, the method comprising:
(a) applying a first centrifugal force and a first flow rate to a cell composition comprising T cells in a conical fluid enclosure of a centrifuge system to produce a fluidized bed of cells, wherein the cell composition comprises viable and non-viable T cells,
(b) applying a second centrifugal force and a second flow rate to the cell composition, wherein the second centrifugal force and second flow rate recirculate cells of the cell composition in a fluid path of the centrifuge system, thereby elutriating out of the conical fluid enclosure a waste fraction of the cell composition that has a higher percentage of nonviable T cells than the percentage of nonviable T cells in the cell composition and generating within the conical fluid enclosure an enriched composition that has a higher percentage of viable T cells than the percentage of viable T cells in the cell composition, and
(c) cryopreserving cells of the enriched composition to create a cryopreserved cell composition following steps (a) and (b).

79. The method of claim 78, further comprising (d) thawing the cryopreserved cell composition following step (c).

80. The method of claim 78 or claim 79, wherein the second flow rate is 30 mL/min or less.

81. The method of any one of claims 78-80, wherein the second flow rate is between about 25 mL/min and about 30 mL/min.

82. A method of enriching a cell composition for viable cells, the method comprising:
(a) applying a first centrifugal force and a first flow rate to a cell composition comprising T cells in a conical fluid enclosure of a centrifuge system to produce a fluidized bed of cells, wherein the cell composition comprises viable and non-viable T cells, wherein (i) the first centrifugal force is between about 2,000 G and about 4,000 G; and (ii) the first flow rate is between about 5 mL/min and about 15 mL/min; and
(b) applying a second centrifugal force and a second flow rate to the cell composition, wherein the second centrifugal force and second flow rate recirculate cells of the cell composition in a fluid path of the centrifuge system, thereby generating an enriched composition having a higher percentage of viable T cells than the percentage of viable T cells in the cell composition, wherein (i) the second centrifugal force is between about 500 G and about 1,500 G; and (ii) the second flow rate is between about 25 mL/min and about 30 mL/min.

83. The method of claim 82, wherein the T cells have a mean diameter of about 9 μm to about 20 μm.

84. The method of claim 82 or claim 83, wherein the method comprises cryopreserving cells of the enriched composition following application of steps (a) and (b) to create a cryopreserved cell composition.

85. The method of any one of claims 78-81 and 84, wherein the cryopreserving comprises suspending the cells in a medium comprising a cryoprotectant and freezing the cells, optionally wherein the freezing is in a controlled rate freezer.

86. The method of any one of claims 78-81, 84, and 85, further comprising thawing the cryopreserved cell composition, optionally wherein the thawing is done after the cryopreserved cell composition has been frozen for at least 3 days.

87. The method of any one of claims 78-86, wherein the second centrifugal force is between about 700 G and about 1,300 G.

88. The method of any one of claims 78-87, wherein the second centrifugal force is between about 800 G and about 1,200 G.

89. The method of any one of claims 78-88, wherein the second centrifugal force is between about 900 G and about 1,100 G.

90. The method of any one of claims 78-89, wherein the second centrifugal force is about 1,000 G.

91. The method of any one of claims 78-90, wherein the ratio of the second centrifugal force (in G) to the second flow rate (in mL/min) is between about 30 and about 40.

92. The method of any one of claims 55-91, wherein the method comprises loading the cell composition into the centrifuge system, wherein the loading is performed prior to and/or during at least a portion of the applying in (a).

93. The method of any one of claims 55-92, wherein the centrifuge system is a continuous counterflow centrifuge system.

94. The method of any one of claims 55-93, wherein prior to the applying in (a), the method comprises contacting the T cells of the cell composition with a viral vector particle, thereby producing genetically engineered T cells and/or the T cells of the cell composition have been contacted with a viral vector particle, thereby producing genetically engineered T cells.

95. The method of any one of claims 55-94, wherein the percentage of viable T cells in the enriched composition is at least about 10% greater, at least about 20% greater, at least about 30% greater, at least about 40% greater, at least about 50% greater, or at least about 60% greater than the percentage of viable T cells in the cell composition.

96. The method of any one of claims 55-95, comprising (c) applying a third centrifugal force and a third flow rate to the enriched composition in the conical fluid enclosure of the centrifuge system to collect the enriched composition, wherein (i) the third centrifugal force is between about 2,000 G and about 3,000 G; and (ii) the third flow rate is between about 15 mL/min and about 25 mL/min.

97. The method of claim 96, wherein prior to the applying the third centrifugal force and the third flow rate, the method comprises subjecting the enriched composition to one or more washing steps, optionally wherein the one or more washing steps comprise media exchange.

98. The method of any one of claims 28-54 and 96, wherein the one or more washing steps are carried out at the second centrifugal force and the second flow rate.

99. The method of any one of claims 1-98, wherein the cell composition comprises activated T cells.

100. The method of any one of claims 55-99, wherein the method comprises cryopreserving the collected enriched composition and/or the collected enriched composition is cryopreserved, thereby generating a cryopreserved enriched composition.

101. The method of claim 100, wherein the cryopreserved enriched composition is thawed to produce a thawed enriched composition, and the percentage of viable T cells in the thawed enriched composition is greater than the percentage of viable T cells in the cell composition, optionally at least about 5% greater, at least about 10% greater, at least about 15% greater, at least about 20% greater, at least about 25% greater, or at least about 30% greater.

102. The method of any one of claims 1-101, wherein one or more steps of the method are automated, optionally wherein the one or more steps of the method are automated by the centrifuge system or a component thereof.

103. The method of any one of claims 1-54 and 94-102, wherein the viral vector particle comprises a heterologous nucleic acid encoding a recombinant molecule.

104. The method of claim 103, wherein the recombinant molecule is a chemokine, a chemokine receptor, a cytokine, a cytokine receptor, an antigen receptor, or a combination thereof.

105. The method of claim 103 or claim 104, wherein the recombinant molecule is an antigen receptor.

106. The method of claim 105, wherein the antigen receptor is a transgenic T cell receptor (TCR).

107. The method of claim 105, wherein the antigen receptor is a chimeric antigen receptor (CAR).

108. The method of claim 107, wherein the chimeric antigen receptor (CAR) comprises an extracellular antigen-recognition domain that specifically binds to a target antigen and an intracellular signaling domain comprising an immunoreceptor tyrosine-based activation motif (ITAM).

109. The method of claim 108, wherein the intracellular signaling domain comprises an intracellular domain of a CD3-zeta (CD3ζ) chain.

110. The method of claim 108 or claim 109, wherein the CAR further comprises a transmembrane domain linking the extracellular domain and the intracellular signaling domain.

111. The method of claim 110, wherein the transmembrane domain comprises a transmembrane portion of CD28.

112. The method of any one of claims 108-111, wherein the intracellular signaling domain further comprises an intracellular signaling domain of a T cell costimulatory molecule.

113. The method of claim 112, wherein the T cell costimulatory molecule is selected from the group consisting of CD28 and 4-1BB.

114. The method of any one of claims 1-54 and 94-113, wherein the viral vector particle is a retroviral vector particle.

115. The method of claim 114, wherein the retroviral vector particle is a γ-retroviral vector.

116. The method of claim 114, wherein the retroviral vector particle is a lentiviral vector particle.

117. The method of any one of claims 105-116, wherein the antigen receptor specifically binds to an antigen associated with a disease or a condition.

118. The method of claim 117, wherein the disease or condition is a cancer, an autoimmune disease or disorder, and/or an infectious disease.

119. The method of claim 117 or claim 118, wherein the disease or condition is a cancer.

120. The method of any one of claims 1-119, wherein the T cells are primary T cells, optionally from a human subject.

121. A composition comprising genetically engineered T cells produced by the method of any one of claims 1-120.

122. The composition of claim 121, wherein the composition comprises between about 1.0×10⁶ CAR-expressing T cells and 2.0×10⁹ CAR-expressing T cells.

123. The composition of claim 121 or claim 122, further comprising a pharmaceutically acceptable carrier.

124. The composition of claim 121 or claim 122, further comprising a cryoprotectant.

125. A method of treating a subject having a disease or disorder, the method comprising administering the composition of any one of claims 121-123 to the subject.

126. The method of claim 125, wherein the genetically engineered T cells express an antigen receptor that specifically binds to an antigen associated with the disease or disorder.
